(19) United States
(12) Patent Application Publication
(10) Pub. No.: US 2012/0156130 A1
(43) Pub. Date: Jun. 21, 2012

(54) USE OF HER3 BINDING AGENTS IN PROSTATE TREATMENT

(76) Inventors: Thore HETTMANN, Munchen (DE); Daniel J. Freeman, Newbury Park, CA (US); Robert Radinsky, Thousand Oaks, CA (US); Darrin M. Beaupre, Simi Valley, CA (US)

(21) Appl. No.: 13/205,608

(22) Filed: Aug. 8, 2011

Related U.S. Application Data
(60) Provisional application No. 61/401,040, filed on Aug. 6, 2010.

(51) Int. Cl. A61K 39/395; A61P 13/08; A61K 51/10

(52) U.S. Cl. 424/1.49; 424/158.1; 424/143.1; 424/133.1; 424/135.1; 424/178.1

(57) ABSTRACT

Described herein are materials and methods for using a HER3 binding agent for prostate treatment. The HER3 binding agent can be, for example, an antibody, and can be used to treat conditions such as benign prostate hyperplasia (BPH) and prostate cancer.

### CROSS REFERENCE TO RELATED APPLICATIONS

This utility patent application claims priority under 35 U.S.C. §119(e) to previously filed and commonly owned Provisional Patent Application No. 61/401,040, filed Aug. 6, 2010.

### REFERENCE TO SEQUENCE LISTING, A TABLE, OR A COMPUTER PROGRAM LISTING COMPACT DISC APPENDIX

A Sequence Listing is being submitted in ASCII character and file format in compliance with 37 CFR §1.52(e) on a properly labeled CD in compliance with 37 CFR §1.96. A copy of the Sequence Listing is also being submitted in computer readable form (CRF) in accordance with the requirements of §1.824. The sequence listing information recorded in computer readable form is identical to the written (on paper or compact disc) sequence listing.

### TECHNICAL FIELD

This document relates to materials and methods for treating prostate conditions, and more particularly to materials and methods for reducing HER3 activity in prostate tissue to treat conditions such as benign prostate hyperplasia (BPH) and prostate cancer.

### BACKGROUND OF THE INVENTION

Prostate cancer patients in the United States undergo radical prostatectomy or radiation therapy for treatment of localized prostate cancer. It is estimated that >15-20% of these patients experience tumor recurrence due to metastasis (See: Melamed et al., “Prostate Cancer Pathologic Parameters and Clinical Outcome: Results from the Cooperative Prostate Cancer Tissue Resource,” National Cancer Institute, Bethesda, Md. (2004)). Recurrent prostate cancer typically is treated with androgen withdrawal therapy, which often is initially effective but ultimately fails, indicating development of androgen independent prostate cancer (AIPC) (See: Petrylak, Brit. J. Urol. Int. 96 (Suppl 2):41-46 (2005)). Treatment options for patients who fail AWT are limited (See: Javidan et al., Cancer Invest. 23(6):520-528 (2005)).

### BRIEF SUMMARY OF THE INVENTION

This document is based in part on the discovery that androgen ablation is accompanied by an increase in the levels of the receptor tyrosine kinase HER3, which stimulates Akt phosphorylation and induces the ability of cells to propagate in medium without androgens. The materials and methods provided herein relate, in part, to using a HER3 binding agent (e.g., a fully human anti-HER3 monoclonal antibody such as U1-59) during androgen withdrawal to inhibit HER3, thus preventing androgen independence.

This document also is based in part on the discovery that intravenous (i.v.) treatment of rats with a HER3 binding agent, such as a HER3 binding antibody, resulted in reduced prostate weight. Thus, this document provides materials and methods for decreasing prostate weight (e.g., to treat conditions such as BPH) with a HER3 binding agent.

The present document describes methods for treating benign prostate hyperplasia (BPH) in a subject in need thereof, comprising administering to the subject a therapeutically effective amount of a composition comprising a HER3 binding agent. The HER3 binding agent may a small molecule compound or an antigen-binding protein that binds to HER3.

In one embodiment, the HER3 binding agent is an antigen-binding protein that binds to HER3, and comprises a heavy chain amino acid sequence that comprises a CDRH1 selected from the group consisting of, for example, SEQ ID NOs: 236, 251, 252, and 256; a CDRH2 selected from the group consisting of, for example, SEQ ID NOs:258, 278, 280, and 282; and a CDRH3 selected from the group consisting of, for example, SEQ ID NOs:283, 285, 309, 313, and 315; and a light chain amino acid sequence that comprises a CDRL1 selected from the group consisting of, for example, SEQ ID NOs: 320, 334, 337, and 340; a CDRL2 selected from the group consisting of, for example, SEQ ID NOs: 343, 356, 351, and 344; and a CDRL3 selected from the group consisting of, for example, SEQ ID NOs:360, 381, 385, and 387.

In another embodiment, the HER3 binding agent is an antigen-binding protein that binds to HER3, and comprises a heavy chain amino acid sequence that comprises at least one of the CDR's selected from the group consisting of (a) CDRH1's as shown in, for example, SEQ ID NOs: 236, 251, 252, and 256; (b) CDRH2's as shown in, for example, SEQ ID NOs: 258, 278, 280, and 282; and (c) CDRH3's as shown in, for example, SEQ ID NOs: 283, 285, 309, 313, and 315.

In a further embodiment, the HER3 binding agent is an antigen-binding protein that binds to HER3, and comprises a light chain amino acid sequence that comprises at least one of the CDR's selected from the group consisting of: (d) CDRL1's as shown in, for example, SEQ ID NOs: 320, 334, 337, and 340; (e) CDRL2's as shown in, for example, SEQ ID NOs: 343, 356, 351, and 344; and (f) CDRL3's as shown in, for example, SEQ ID NOs: 360, 381, 385, and 387.

In yet another embodiment, the HER3 binding agent is an antigen-binding protein that binds to HER3, and comprises a heavy chain amino acid sequence that comprises at least one of the CDR's selected from the group consisting of, for example, (a) CDRH1's as shown in SEQ ID NOs: 236, 251, 252, and 256; (b) CDRH2's as shown in SEQ ID NOs: 258, 278, 280, and 282; and (c) CDRH3's as shown in SEQ ID NOs: 283, 285, 309, 313, and 315; and a light chain amino acid sequence that comprises at least one of the CDR's selected from the group consisting of: (d) CDRL1's as shown in SEQ ID NOs: 320, 334, 337, and 340; (e) CDRL2's as shown in SEQ ID NOs: 343, 356, 351, and 344; and (f) CDRL3's as shown in SEQ ID NOs:360, 381, 385, and 387.

In yet another embodiment, the HER3 binding agent is an antigen-binding protein that binds to HER3, and comprises a heavy chain amino acid sequence that comprises a CDRH1 selected from the group consisting of, for example, SEQ ID NOs: 236, 251, 252, and 256, a CDRH2 selected from the group consisting of, for example, SEQ ID NOs: 258, 278, 280, and 282, and a CDRH3 selected from the group consisting of, for example, SEQ ID NOs: 283, 285, 309, 313, and 315, or a light chain amino acid sequence that comprises a CDRL1 selected from the group consisting of, for example, SEQ ID NOs: 320, 334, 337, and 340, a CDRL2 selected from the group consisting of, for example, SEQ ID NOs: 343, 356, 351, and 344, and a CDRL3 selected from the group consisting of, for example, SEQ ID NOs: 360, 381, 385, and 387.

In another embodiment, the HER3 binding agent is an antigen-binding protein that binds to HER3, and comprises a heavy chain amino acid sequence selected from the group consisting of, for example, SEQ ID NOs: 42, 54, 70, 92, and 96. Alternatively, the antigen-binding protein comprises a light chain amino acid sequence selected from the group consisting of, for example, SEQ ID NOs: 44, 56, 72, 94, and 98. In some embodiments, the HER3 binding agent is an antigen-binding protein that binds to HER3, and comprises a heavy chain amino acid sequence selected from the group consisting of, for example, SEQ ID NOs: 42, 54, 70, 92, and 96; and a light chain amino acid sequence selected from the group consisting of, for example, SEQ ID NOs: 44, 56, 72, 94, and 98. The HER3 binding agent may comprise the heavy chain amino acid sequence of SEQ ID NO: 42 and the light chain amino acid sequence of SEQ ID NO: 44, or the heavy chain amino acid sequence of SEQ ID NO: 54 and the light chain amino acid sequence of SEQ ID NO:56, or the heavy chain amino acid sequence of SEQ ID NO: 70 and the light chain amino acid sequence of SEQ ID NO: 72.

In some embodiments, the HER3 binding agent is an antigen-binding protein that binds to HER3, and comprises a CDRH3 selected from the group consisting of, for example, SEQ ID NOs: 283, 285, 309, 313, and 315. In other embodiments, the HER3 binding agent is an antigen-binding protein that binds to HER3, and comprises a CDHL3 selected from the group consisting of, for example, SEQ ID NOs: 360, 381, 385, and 387.

In certain examples, the antigen-binding protein is directed against the extracellular domain of HER3. The binding of the antigen-binding protein to HER3 may have one or more effects selected from the group consisting of reduction of HER3-mediated signal transduction, reduction of HER3 phosphorylation, reduction of cell proliferation, reduction of cell migration, and increasing downregulation of HER3.

In certain embodiments, the antigen-binding protein that binds to HER3 is an antibody. The antibody may be a monoclonal antibody, a polyclonal antibody, a recombinant antibody, a humanized antibody, a human antibody, a chimeric antibody, a multispecific antibody, or an antibody fragment thereof. The antibody fragment may be a Fab fragment, a Fab′ fragment, a F(ab′)2 fragment, a Fv fragment, a diabody, or a single chain antibody molecule. For example, the antibody is of the IgG1-, IgG2-, IgG3- or IgG4-type.]

In some embodiments, the antigen-binding protein is coupled to an effector group. The effector group may be a radioisotope or radionuclide, a toxin, or a therapeutic or chemotherapeutic group. The therapeutic or chemotherapeutic group may be selected from the group consisting of calicheamicin, auristatin-PE, geldanamycin, maytansine and derivatives thereof.

The method of the invention may include identifying the subject as having BPH, and/or monitoring prostate size in the subject after administering the composition.

The present document also relates to methods for reducing prostate weight in a subject, comprising administering to the subject a therapeutically effective amount of a composition comprising a HER3 binding agent. The HER3 binding agent may be a small molecule compound or an antigen-binding protein that binds to HER3.

In one embodiment, the HER3 binding agent is an antigen-binding protein that binds to HER3, and comprises a heavy chain amino acid sequence that comprises a CDRH1 selected from the group consisting of, for example, SEQ ID NOs: 236, 251, 252, and 256; a CDRH2 selected from the group consisting of, for example, SEQ ID NOs: 258, 278, 280, and 282; and a CDRH3 selected from the group consisting of, for example, SEQ ID NOs: 283, 285, 309, 313, and 315; and a light chain amino acid sequence that comprises a CDRL1 selected from the group consisting of, for example, SEQ ID NOs: 320, 334, 337, and 340; a CDRL2 selected from the group consisting of, for example, SEQ ID NOs: 343, 356, 351, and 344; and a CDRL3 selected from the group consisting of, for example, SEQ ID NOs: 360, 381, 385, and 387.

In other embodiments, the HER3 binding agent is an antigen-binding protein that binds to HER3, and comprises a heavy chain amino acid sequence that comprises at least one of the CDR's selected from the group consisting of, for example, (a) CDRH1's as shown in SEQ ID NOs: 236, 251, 252, and 256; (b) CDRH2's as shown in SEQ ID NOs: 258, 278, 280, and 282; and (c) CDRH3's as shown in SEQ ID NOs: 283, 285, 309, 313, and 315.

In yet other embodiments, the HER3 binding agent is an antigen-binding protein that binds to HER3, and comprises a light chain amino acid sequence that comprises at least one of the CDR's selected from the group consisting of, for example, (d) CDRL1's as shown in SEQ ID NOs: 320, 334, 337, and 340; (e) CDRL2's as shown in SEQ ID NOs: 343, 356, 351, and 344; and (f) CDRL3's as shown in SEQ ID NOs: 360, 381, 385, and 387.

In yet other embodiments, the HER3 binding agent is an antigen-binding protein that binds to HER3, and comprises a heavy chain amino acid sequence that comprises at least one of the CDR's selected from the group consisting of, for example, (a) CDRH1's as shown in SEQ ID NOs: 236, 251, 252, and 256; (b) CDRH2's as shown in SEQ ID NOs: 258, 278, 280, and 282; and (c) CDRH3's as shown in SEQ ID NOs: 283, 285, 309, 313, and 315; and a light chain amino acid sequence that comprises at least one of the CDR's selected from the group consisting of: (d) CDRL1's as shown in SEQ ID NOs: 320, 334, 337, and 340; (e) CDRL2's as shown in SEQ ID NOs: 343, 356, 351, and 344; and (f) CDRL3's as shown in SEQ ID NOs: 360, 381, 385, and 387.

In again other embodiment, the HER3 binding agent is an antigen-binding protein that binds to HER3, and comprises a heavy chain amino acid sequence that comprises a CDRH1 selected from the group consisting of, for example, SEQ ID NOs: 236, 251, 252, and 256, a CDRH2 selected from the group consisting of, for example, SEQ ID NOs: 258, 278, 280, and 282, and a CDRH3 selected from the group consisting of, for example, SEQ ID NOs: 283, 285, 309, 313, and 315, or a light chain amino acid sequence that comprises a CDRL1 selected from the group consisting of, for example, SEQ ID NOs: 320, 334, 337, and 340, a CDRL2 selected from the group consisting of, for example, SEQ ID NOs: 343, 356, 351, and 344, and a CDRL3 selected from the group consisting of, for example, SEQ ID NOs: 360, 381, 385, and 387.

In certain embodiments, the HER3 binding agent is an antigen-binding protein that binds to HER3, and comprises a heavy chain amino acid sequence selected from the group consisting of, for example, SEQ ID NOs: 42, 54, 70, 92, and 96. Alternatively, the antigen-binding protein comprises a light chain amino acid sequence selected from the group consisting of, for example, SEQ ID NOs: 44, 56, 72, 94, and 98.

The HER3 binding agent may comprise a heavy chain amino acid sequence selected from the group consisting of, for example, SEQ ID NOs: 42, 54, 70, 92, and 96; and a light chain amino acid sequence selected from the group consisting of, for example, SEQ ID NOs: 44, 56, 72, 94, and 98.

The HER3 binding agent may be an antigen-binding protein that binds to HER3, and comprises the heavy chain amino acid sequence of SEQ ID NO: 42 and the light chain amino acid sequence of SEQ ID NO: 44. Alternatively, the HER3 binding agent is an antigen-binding protein that binds to HER3, and comprises the heavy chain amino acid sequence of SEQ ID NO: 54 and the light chain amino acid sequence of SEQ ID NO:56. Again, alternatively, the HER3 binding agent is an antigen-binding protein that binds to HER3, and comprises the heavy chain amino acid sequence of SEQ ID NO: 70 and the light chain amino acid sequence of SEQ ID NO: 72.

In certain embodiments, the HER3 binding agent is an antigen-binding protein that binds to HER3, and comprises a CDRH3 selected from the group consisting of, for example, SEQ ID NOs: 283, 285, 309, 313, and 315. In yet other embodiments, the HER3 binding agent is an antigen-binding protein that binds to HER3, and comprises a CDHL3 selected from the group consisting of, for example, SEQ ID NOs: 360, 381, 385, and 387.

In certain embodiments, the antigen-binding protein is directed against the extracellular domain of HER3. The binding of the antigen-binding protein to HER3 may have one or more effects selected from the group consisting of reduction of HER3-mediated signal transduction, reduction of HER3 phosphorylation, reduction of cell proliferation, reduction of cell migration, and increasing downregulation of HER3.

In certain embodiments, the antigen-binding protein that binds to HER3 is an antibody. The antibody may be a monoclonal antibody, a polyclonal antibody, a recombinant antibody, a humanized antibody, a human antibody, a chimeric antibody, a multispecific antibody, or an antibody fragment thereof. The antibody fragment may be a Fab fragment, a Fab′ fragment, a F(ab′)2 fragment, a Fv fragment, a diabody, or a single chain antibody molecule. For example, the antibody is of the IgG1-, IgG2-, IgG3- or IgG4-type.

In some embodiments, the antigen-binding protein is coupled to an effector group. The effector group may be a radioisotope or radionuclide, a toxin, or a therapeutic or chemotherapeutic group. The therapeutic or chemotherapeutic group may be, for example, a calicheamicin, a auristatin-PE, a geldanamycin, a maytansine or derivatives thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. Publications, patent applications, patents, and other references mentioned herein are hereby incorporated herein by reference in their entireties. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE SEVERAL VIEWS OF THE DRAWING

FIGS. 1A and 1B are pictures of blots showing ErbB levels in various cell lines. FIG. 1C is a graph plotting luciferase activity in LNCaP cells transfected with a luciferase construct tagged to a PSA promoter, indicating the effects of culture in medium containing charcoal stripped serum (CSS) on androgen receptor (AR) transcriptional activity. FIG. 1D is a picture of a blot showing expression levels of various proteins after culture of LNCaP cells in medium containing CSS.

FIG. 2A is a picture of a blot showing levels of several proteins after over-expression of HER2 and HER3 in LNCaP cells. Overexpression of HER3 increased HER2 expression. FIG. 2B is a graph plotting LNCaP growth rates in CSS-containing medium for control cells, cells overexpressing HER2 or HER3, and an androgen independent (AI) subline. Both HER2 and HER3 caused LNCaP cells to grow in medium containing CSS. FIG. 2C is a graph plotting growth rates for the LNCaP AI subline cultured with fetal bovine serum (FBS) or CSS, with control or HER3 siRNA. Inhibition of HER3 in the AI subline prevented this cell growth in CSS-containing medium. FIG. 2D is a graph plotting growth of LNCaP cells with or without HER3 overexpression, with or without an Akt inhibitor. The HER3-induced increase in cell growth was prevented by Akt inhibition.

FIG. 3A is a graph plotting the percentage of cells in S-phase for mock transfected LNCaP and C4-2 cells (control cells were treated with LIPOFECATMINE® 2000 alone), or cells transfected with scrambled or HER3-specific siRNA duplex. Bars represent mean±S.E. from three individual experiments. *p<0.01 in siRNA transfected vs. mock transfected cells. FIG. 3B is a picture of a Western blot and a graph plotting band intensity to indicate HER3 protein levels in LNCaP and C4-2 cells incubated with scrambled or HER3 siRNA. HER2 (HER2) was used as loading control. Bars represent mean±S.E. of band intensity quantization of HER3 bands normalized to HER2 bands from three individual experiments. FIG. 3C is a graph plotting the percentage of LNCaP cells in S-phase after transfection with empty vector (pcDNA3) or pcDNA3-HER3-myc cDNA and treatment with bicalutamide (an AR antagonist marketed as CASODEX® by AstraZeneca, Wilmington, Del.) or DMSO (control). Bars represent mean±S.E. of S-phase percentages from three individual experiments. *p<0.01 in bicalutamide treated vs. untreated cells. FIG. 3D is a picture of a Western blot and a graph plotting protein levels of HER3 in LNCaP or C4-2 cells transfected with control (empty pCNDA3 vector) or pcDNA3-HER3-myc cDNA, confirming overexpression of HER3 in the appropriate cells.

FIGS. 4A and 4B are pictures of western blots using lysates from DU-145 prostate cancer cells treated with IgG, U1-59, cetuximab, panitumumab, c2C4, trastuzumab, or combinations thereof, as indicated. Blots were probed with antibodies against HER3 phosphorylated at Tyrosine 1289 or 1197. Actin was used as a control. FIGS. 4A and 4B represent separate experiments.

FIG. 5 is a graph plotting colony numbers for PC-3 prostate cancer cells incubated with IgG, U1-59, or either of two other anti-HER3 antibodies (U1-53 and U1-49), as indicated.

FIGS. 6A and 6B are pictures of western blots showing phospho-HER3 levels in rat RG2 glioma cells (FIG. 6A) and cynomolgus monkey JTC-1.P3 cells (FIG. 6B) incubated with heregulin, U1-59, IgG control, or combinations thereof, as indicated. -actin was used as a control for loading.

### DETAILED DESCRIPTION OF THE INVENTION

### 1. General Overview

This document provides materials and methods related to treating prostate conditions such as BPH and prostate cancer. Unless otherwise defined herein, scientific and technical terms used in connection with this document shall have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques described herein generally are performed according to conventional methods well known in the art and as described in various general and more specific references that are cited and discussed throughout the present specification unless otherwise indicated. See: Sambrook et al., Molecular Cloning: A Laboratory Manual, 3rd ed., Cold Spring Harbor Laboratory Press, Cold Spring Harbor, N.Y. (2001); Ausubel et al., Current Protocols in Molecular Biology, Greene Publishing Associates (1992); and Harlow and Lane Antibodies: A Laboratory Manual Cold Spring Harbor Laboratory Press, Cold Spring Harbor, N.Y. (1990), which publications are hereby incorporated herein by reference in their entireties. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

It should be understood that this invention is not limited to the particular methodology, protocols, reagents, etc., described herein and as such may vary. The terminology used herein is for the purpose of describing particular embodiments only, and is not intended to limit the scope of the disclosed, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

### 2. The Prostate and HER Family Members

Androgen withdrawal therapy (AWT) for treatment of prostate cancer does not induce cell death, but rather results in cell cycle arrest. The development of androgen independence triggers a release from that arrest, leading to cell cycle progression even in the absence of androgens (See: Agus et al., J. Natl. Cancer Inst. 91(21):1869-1876 (1999)). Some of the studies described herein were conducted to determine whether induction of apoptosis during AWT can prevent androgen independent cell cycle progression.

Androgen withdrawal is accompanied by an increase in phosphorylation (activation) of Akt (See: Mikhailova et al. in: Hormonal Carcinogenesis V, Springer Science and Business Media, New York, N.Y., pp. 397-405 (2008); and Murillo et al. Endocrinol. 142(11):4795-4805 (2001)), a serine threonine kinase that promotes cell survival (See: Graff et al., J. Biol. Chem. 275(32):24500-24505 (2000); and Xin et al., Proc. Natl. Acad. Sci. USA 103(20):7789-7794 (2006)). Direct inhibition of Akt phosphorylation may cause some toxicity since Akt is an essential kinase (See: Posadas et al., Cancer Biol. Ther. 4(10):1133-1137 (2005); and Chee et al., Clin. Genitourin Cancer 5(7):433-437 (2007)). The inventors hypothesized that inhibition of pathways stimulating Akt phosphorylation during androgen withdrawal would prevent cell survival and eventually prevent the emergence of androgen independent cells.

The HER family of proteins, which activate Akt in prostate epithelial cells, includes the epidermal growth factor receptor (EGFR, also known as HER1 and ErbB1), HER2 (also known as ErbB2 and neu), HER3 (also known as ErbB3), and HER4 (also known as ErbB4) (See: Olayioye et al., EMBO J. 19:3159-3167 (2000)). EGFR and HER2 regulate cell proliferation, differentiation, angiogenesis, and survival (See: Yarden and Sliwkowski, Nat. Rev. Mol. Cell. Biol. 2(2):127-137 (2001)). Less is known about HER3 and HER4, but microarray analysis revealed increased expression of the HER3 gene in prostate cancer vs. normal prostate (See: Chaib et al. Neoplasia 3(1):43-52 (2001)), and immunohistochemistry analysis of prostate cancer tissues revealed that >90% of displayed cytoplasmic HER3 staining (See: Koumakpayi et al., Clin. Cancer Res. 12(9):2730-2737 (2006)). Further, activation of HER3 in an animal model induced activation of the AR and promoted prostate cancer recurrence (See: Gregory et al., Clin. Cancer Res. 11(5):1704-1712 (2005)). Thus, increased HER3 levels may promote prostate cancer progression.

The HER family receptors are activated by ligand binding, dimerization and phosphorylation. Each receptor except HER2 has a specific ligand (See: Olayioye et al., EMBO J. 19(13):3159-3167 (2000)). As such, HER2 requires heterodimerization with other ErbB receptors for phosphorylation and activation. HER3 also requires heterodimerization with other ErbB receptors for kinase activity. Despite this, HER3 has multiple phosphorylation sites that specifically bind and activate downstream targets. In particular, HER3 has six binding sites for phosphatidylinositol 3-kinase (PI3K), an upstream activator required for the phosphorylation and activation of Akt. EGFR and HER2 can also activate Akt independent of HER3, but this activation is indirect, via transactivation of PI3K by Src kinases.

BPH is an enlargement of the prostate gland, and is very common in older men due to continued growth of the prostate throughout life. Although BPH is noncancerous, it can lead to urinary problems such as urinary tract infection, bladder stones, blood in the urine and, in some extreme cases, kidney failure. Treatment for BPH can range from changes in diet and exercise or use of medication in mild cases, to destruction of overgrown tissue (e.g., using microwave thermal therapy, laser therapy, transurethral needle ablation, or other minimally invasive therapies) in moderate cases, to transuretheral prostate resection, or to open surgery in severe cases.

### 3. HER3 Binding Agents

As described herein, an agent that binds to HER3 can be a biological compound, including, but not limited to, an antigen binding protein (e.g., an antibody) or a small molecular tyrosine kinase inhibitor. As used herein, an “antigen binding protein” or “binding protein” means a protein that specifically binds a particular target antigen, such as member of the HER family, e.g., HER3. An antigen binding protein is understood to “specifically bind” its target antigen when the dissociation constant (K_{D}) is ≦10⁻⁸ M. The antibody specifically binds antigen with “high affinity” when the K_{D} is ≦5×10⁻⁹ M, and with “very high affinity” when the K_{D} is ≦5×10⁻¹⁰ M. In one embodiment, the antibody has a K_{D} of ≦10⁻⁹ M and an off-rate of about 1×10⁻⁴/sec. In one embodiment, the off-rate is about 1×10⁵/sec. In other embodiments, the antibodies will bind to a specified member of the HER family with a K_{D} of between about 10⁻⁸ M and 10⁻¹⁰ M, and in yet another embodiment it will bind with a K_{D}≦2×10⁻¹⁰. Further, as used herein, a small molecule compound is a low molecular weight compound that has been chemically synthesized to inhibit the enzymatic activity of one or more protein kinases, including serine, threonine, or tyrosine kinases.

Where the HER3 binding agent is a biological compound, the agent can be an antigen binding protein (e.g., an anti-HER3 antibody). Thus, provided herein for use in compositions and methods of treating HER3 associated diseases are HER binding proteins, including anti-HER3 antibodies. In some embodiments, an antibody targeted to HER3 can be directed against the extracellular domain (ECD) of HER3. For example, an anti-HER3 antibody as described herein can interact with at least one epitope in the extracellular part of HER3. The epitopes can be located in the amino terminal L1 domain (aa 19-184), in the S1 (aa 185-327) and S2 (aa 500-632) cysteine-rich domains, in the L2 domain (328-499), which is flanked by the two cysteine-rich domains, or in a combination of HER3 domains. The epitopes also may be located in combinations of domains such as, without limitation, an epitope comprised by parts of L1 and S1.

A HER3 binding protein can be further characterized in that its binding to HER3 reduces HER3-mediated signal transduction. A reduction of HER3-mediated signal transduction may, e.g., be caused by a downregulation of HER3 resulting in an at least partial disappearance of HER3 molecules from the cell surface or by a stabilization of HER3 on the cell surface in a substantially inactive form, i.e., a form that exhibits a lower signal transduction compared to the non-stabilized form. Alternatively, a reduction of HER3-mediated signal transduction also may be caused by influencing, e.g., decreasing or inhibiting, the binding of a ligand or another member of the HER family to HER3. For example, a reduction of HER3 mediated signal transduction also can be caused by, decreasing the formation of HER3 containing dimers with other HER family members (e.g., EGF-R).

A HER3 binding agent can be a scaffold protein having an antibody-like binding activity (e.g., having activity similar to an anti-HER3 antibody) or an antibody, i.e., an anti-HER3 antibody. As used herein, the term “scaffold protein” means a polypeptide or protein with exposed surface areas in which amino acid insertions, substitutions or deletions are highly tolerable. Examples of scaffold proteins that can be used in accordance with the present methods include protein A from Staphylococcus aureus, the bilin binding protein from Pieris brassicae or other lipocalins, ankyrin repeat proteins, and human fibronectin (reviewed in Binz and Plückthun, Curr. Opin. Biotechnol. 16:459-69 (2005)). Engineering of a scaffold protein can be regarded as grafting or integrating an affinity function onto or into the structural framework of a stably folded protein. Affinity function means a protein binding affinity according to the present document. A scaffold can be structurally separable from the amino acid sequences conferring binding specificity. In general, proteins appearing suitable for the development of such artificial affinity reagents may be obtained by rational, or most commonly, combinatorial protein engineering techniques such as panning against HER3, either purified protein or protein displayed on the cell surface, for binding agents in an artificial scaffold library displayed in vitro, skills which are known in the art (See: Skerra, J. Mol. Recog. 13:167-87 (2000); and Binz and Plückthun, supra). In addition, a scaffold protein having an antibody like binding activity can be derived from an acceptor polypeptide containing the scaffold domain, which can be grafted with binding domains of a donor polypeptide to confer the binding specificity of the donor polypeptide onto the scaffold domain containing the acceptor polypeptide. The inserted binding domains may be, for example, the complementarity determining region (CDR) of an antibody, in particular an anti-HER3 antibody. Insertion can be accomplished by various methods known to those skilled in the art including, for example, polypeptide synthesis, nucleic acid synthesis of an encoding amino acid as well by various forms of recombinant methods well known to those skilled in the art.

The term “antibody” includes monoclonal antibodies, polyclonal antibodies, recombinant antibodies, human antibodies, humanized antibodies (See: Jones et al., Nature 321:522-525 (1986); Riechmann et al., Nature 332:323-329 (1988); and Presta, Curr. Op. Struct. Biol. 2:593-596 (1992)), chimeric antibodies (See: Morrison et al., Proc. Natl. Acad. Sci. USA 81:6851-6855 (1984)), multispecific antibodies (e.g., bispecific antibodies) formed from at least two antibodies, or antibody fragments thereof. The term “antibody fragment” comprises any portion of the afore-mentioned antibodies, such as their antigen binding or variable regions. Examples of antibody fragments include Fab fragments, Fab′ fragments, F(ab′)₂ fragments, Fv fragments, diabodies (See: Hollinger et al., Proc. Natl. Acad. Sci. USA 90:6444-6448 (1993)), single chain antibody molecules (See: Plückthun: The Pharmacology of Monoclonal Antibodies 113, Rosenburg and Moore, eds., Springer Verlag, N.Y. 269-315, (1994)), and other fragments as long as they exhibit the desired capability of binding to HER3.

In addition, the term “antibody,” as used herein, also includes antibody-like molecules that contain engineered sub-domains of antibodies or naturally occurring antibody variants. These antibody-like molecules may be single-domain antibodies such as V_{H}-only or V_{L}-only domains derived either from natural sources such as camelids (See: Muyldermans et al., Rev. Mol. Biotechnol. 74:277-302 (2001)) or through in vitro display of libraries from humans, camelids or other species (See: Holt et al., Trends Biotechnol. 21:484-90 (2003)). In certain embodiments, the polypeptide structure of the antigen binding proteins can be based on antibodies, including, but not limited to, minibodies, synthetic antibodies (sometimes referred to as “antibody mimetics”), human antibodies, antibody fusions (sometimes referred to as “antibody conjugates”), and fragments thereof, respectively.

An “Fv fragment” is the minimum antibody fragment that contains a complete antigen-recognition and -binding site. This region consists of a dimer of one heavy chain variable domain and one light chain variable domain in tight, non-covalent association. It is in this configuration that the three CDR's of each variable domain interact to define an antigen-binding site on the surface of the V_{H}-V_{L} dimer. Collectively, the six CDR's confer antigen-binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (CH1) of the heavy chain. The “Fab fragment” differs from the “Fab′ fragment” by the addition of a few residues at the carboxy terminus of the heavy chain CH1 domain, including one or more cysteines from the antibody hinge region. The “F(ab′)₂ fragment” originally is produced as a pair of “Fab′ fragments” which have hinge cysteines between them. Methods of preparing such antibody fragments, such as papain or pepsin digestion, are known to those skilled in the art.

An antibody can be of the IgA-, IgD-, IgE, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type.

In certain respects, e.g., in connection with the generation of antibodies as therapeutic candidates against HER3, it may be desirable that the antibody is capable of fixing complement and participating in complement-dependent cytotoxicity (CDC). There are a number of isotypes of antibodies that are capable of the same including: murine IgM, murine IgG2a, murine IgG2b, murine IgG3, human IgM, human IgG1, human IgG3, and human IgA, for example. It will be appreciated that antibodies that are generated need not initially possess such an isotype but, rather the antibody as generated can possess any isotype and the antibody can be isotype switched by appending the molecularly cloned V region genes or cDNA to molecularly cloned constant region genes or cDNAs in appropriate expression vectors using conventional molecular biological techniques that are well known in the art and then expressing the antibodies in host cells using techniques known in the art. The isotype-switched antibody may also possess an Fc region that has been molecularly engineered to possess superior CDC over naturally occurring variants (See: Idusogie et al., J. Immunol. 166:2571-2575 (2001)) and expressed recombinantly in host cells using techniques known in the art. Such techniques include the use of direct recombinant techniques (See: U.S. Pat. No. 4,816,397), cell-cell fusion techniques (See: U.S. Pat. Nos. 5,916,771 and 6,207,418), among others. In the cell-cell fusion technique, a myeloma or other cell line such as CHO is prepared that possesses a heavy chain with any desired isotype and another myeloma or other cell line such as CHO is prepared that possesses the light chain. Such cells can thereafter be fused, and a cell line expressing an intact antibody can be isolated. By way of example, a human anti-HER3 IgG4 antibody that possesses the desired binding to the HER3 antigen can be readily isotype switched to generate a human IgM, human IgG1 or human IgG3 isotype, while still possessing the same variable region (which defines the antibody's specificity and some of its affinity). Such a molecule might then be capable of fixing complement and participating in CDC.

Moreover, an antibody also may be capable of binding to Fc receptors on effector cells such as monocytes and natural killer (NK) cells, and participating in antibody-dependent cellular cytotoxicity (ADCC). There are a number of antibody isotypes that are capable of the same, including, without limitation, the following: murine IgG2a, murine IgG2b, murine IgG3, human IgG1 and human IgG3. It will be appreciated that the antibodies that are generated need not initially possess such an isotype but, rather the antibody as generated can possess any isotype and the antibody can be isotype switched by appending the molecularly cloned V region genes or cDNA to molecularly cloned constant region genes or cDNAs in appropriate expression vectors using conventional molecular biological techniques that are well known in the art and then expressing the antibodies in host cells using techniques known in the art. The isotype-switched antibody may also possess an Fc region that has been molecularly engineered to possess superior ADCC over naturally occurring variants (See: Shields et al., J. Biol. Chem. 276:6591-6604 (2001)) and expressed recombinantly in host cells using techniques known in the art. Such techniques include the use of direct recombinant techniques (See: U.S. Pat. No. 4,816,397), cell-cell fusion techniques (See: U.S. Pat. Nos. 5,916,771 and 6,207,418), among others. In the cell-cell fusion technique, a myeloma or other cell line such as CHO is prepared that possesses a heavy chain with any desired isotype and another myeloma or other cell line such as CHO is prepared that possesses the light chain. Such cells can thereafter be fused, and a cell line expressing an intact antibody can be isolated. By way of example, a human anti-HER3 IgG4 antibody that possesses the desired binding to the HER3 antigen could be readily isotype switched to generate a human IgG1 or human IgG3 isotype, while still possessing the same variable region (which defines the antibody's specificity and some of its affinity). Such molecule might then be capable of binding to FcR on effectors cells and participating in ADCC.

TABLE 1 herein provides amino acid sequences for a number of CDR's that can be included in antibodies against HER3. In some embodiments, an isolated binding protein targeted to HER3 can include a heavy chain amino acid sequence containing at least one CDR selected from the group consisting of: (a) CDRH1's as shown in SEQ ID NOS: 2, 6, 10, 14, 18, 22, 26, 30, 34, 36, 40, 42, 46, 50, 54, 60, 62, 66, 70, 74, 78, 80, 84, 88, 92, 96, 100, 104, 108, 112, 116, 120, 122, 126, 130, 134, 138, 142, 146, 150, 154, 158, 162, 166, 170, 174, 178, 182, 186, 190, 194, 198, 202, 206, 210, 214, 218, 222, 226 and 230, (b) CDRH2's as shown in SEQ ID NOS: 2, 6, 10, 14, 18, 22, 26, 30, 34, 36, 40, 42, 46, 50, 54, 60, 62, 66, 70, 74, 78, 80, 84, 88, 92, 96, 100, 104, 108, 112, 116, 120, 122, 126, 130, 134, 138, 142, 146, 150, 154, 158, 162, 166, 170, 174, 178, 182, 186, 190, 194, 198, 202, 206, 210, 214, 218, 222, 226 and 230, and (c) CDRH3's as shown in SEQ ID NOS: 2, 6, 10, 14, 18, 22, 26, 30, 34, 36, 40, 42, 46, 50, 54, 60, 62, 66, 70, 74, 78, 80, 84, 88, 92, 96, 100, 104, 108, 112, 116, 120, 122, 126, 130, 134, 138, 142, 146, 150, 154, 158, 162, 166, 170, 174, 178, 182, 186, 190, 194, 198, 202, 206, 210, 214, 218, 222, 226 and 230, and/or a light chain amino acid sequence comprising at least one of the CDR's selected from the group consisting of: (d) CDRL1's as shown in SEQ ID NOS: 4, 8, 12, 16, 20, 24, 28, 32, 38, 44, 48, 52, 56, 58, 64, 68, 72, 76, 82, 86, 90, 94, 98, 102, 106, 110, 114, 118, 124, 128, 132, 136, 140, 144, 148, 152, 156, 160, 164, 168, 172, 176, 180, 184, 188, 192, 196, 200, 204, 208, 212, 216, 220, 224, 228 and 232, (e) CDRL2's as shown in SEQ ID NOS: 4, 8, 12, 16, 20, 24, 28, 32, 38, 44, 48, 52, 56, 58, 64, 68, 72, 76, 82, 86, 90, 94, 98, 102, 106, 110, 114, 118, 124, 128, 132, 136, 140, 144, 148, 152, 156, 160, 164, 168, 172, 176, 180, 184, 188, 192, 196, 200, 204, 208, 212, 216, 220, 224, 228 and 232, and (f) CDRL3's as shown in SEQ ID NOS: 4, 8, 12, 16, 20, 24, 28, 32, 38, 44, 48, 52, 56, 58, 64, 68, 72, 76, 82, 86, 90, 94, 98, 102, 106, 110, 114, 118, 124, 128, 132, 136, 140, 144, 148, 152, 156, 160, 164, 168, 172, 176, 180, 184, 188, 192, 196, 200, 204, 208, 212, 216, 220, 224, 228 and 232. All sequences referred to herein are shown in the sequence listing in U.S. Pat. No. 7,705,130. These sequences are hereby incorporated herein by reference in their entireties.

TABLE 1
CDR SEQUENCES

| | | SEQ | | SEQ | | SEQ | |
|---|---|---|---|---|---|---|---|
| Ab | Pat | ID | | ID | | ID | |
| chain | ID | NO | CDR1 | NO | CDR2 | NO | CDR3 |
| heavy | U1-1 | 235 | GGSINSGDYYWS | 258 | YIYYSGSTYYNPSLKS | 283 | ADYDFWSGYFDY |
| light | U1-1 | 318 | RASQGIRNDLG | 343 | AASSLQS | 360 | LQHNSYPWT |
| heavy | U1-2 | 236 | GGSISSGDYYWS | 259 | YIYYSGSTYYNPSLRS | 283 | ADYDFWSGYFDY |
| light | U1-2 | 318 | RASQGIRNDLG | 343 | AASSLQS | 361 | LQHNGYPWT |
| heavy | U1-3 | 237 | GGSISSGGYYWS | 258 | YIYYSGSTYYNPSLKS | 284 | DGYDSSGYYHGYFDY |
| light | U1-3 | 319 | KSSQSVLYSSNNKNYLA | 344 | WASTRES | 362 | QQYYSTPLT |
| heavy | U1-4 | 236 | GGSISSGDYYWS | 258 | YIYYSGSTYYNPSLKS | 283 | ADYDFWSGYFDY |
| light | U1-4 | 318 | RASQGIRNDLG | 343 | AASSLQS | 363 | LQHNNYPWT |
| heavy | U1-5 | 236 | GGSISSGDYYWS | 258 | YIYYSGSTYYNPSLKS | 283 | ADYDFWSGYFDY |
| light | U1-5 | 318 | RASQGIRNDLG | 343 | AASSLQS | 364 | LQHNTYPWT |
| heavy | U1-6 | 236 | GGSISSGDYYWS | 258 | YIYYSGSTYYNPSLKS | 285 | ADYDFWNGYFDY |
| light | U1-6 | 318 | RASQGIRNDLG | 343 | AASSLQS | 364 | LQHNTYPWT |
| heavy | U1-7 | 236 | GGSISSGDYYWS | 258 | YIYYSGSTYYNPSLKS | 283 | ADYDFWSGYFDY |
| light | U1-7 | 320 | RASQDIRNDLG | 343 | AASSLQS | 360 | LQHNSYPWT |
| heavy | U1-8 | 238 | GYTLTELSMY | 260 | GFDPEDGETIYAQKFQG | 286 | GWNYVFDY |
| light | U1-8 | 321 | RSSQSLLHSNGYNYLD | 345 | LDSHRAS | 365 | MQALQTPLT |
| heavy | U1-9 | 236 | GGSISSGDYYWS | 258 | YIYYSGSTYYNPSLKS | 285 | ADYDFWNGYFDY |
| light | U1-9 | 320 | RASQDIRNDLG | 343 | AASSLQS | 360 | LQHNSYPWT |
| heavy | U1-10 | 236 | GGSISSGDYYWS | 258 | YIYYSGSTYYNPSLKS | 283 | ADYDFWSGYFDY |
| light | U1-10 | 318 | RASQGIRNDLG | 343 | AASSLQS | 363 | LQHNNYPWT |
| heavy | U1-11 | 236 | GGSISSGDYYWS | 258 | YIYYSGSTYYNPSLKS | 283 | ADYDFWSGYFDY |
| light | U1-11 | 318 | RASQGIRNDLG | 343 | AASSLQS | 364 | LQHNTYPWT |
| heavy | U1-12 | 236 | GGSISSGDYYWS | 258 | YIYYSGSTYYNPSLKS | 283 | ADYDFWSGYFDY |
| light | U1-12 | 318 | RASQGIRNDLG | 343 | AASSLQS | 363 | LQHNNYPWT |
| heavy | U1-13 | 237 | GGSISSGGYYWS | 258 | YIYYSGSTYYNPSLKS | 287 | EDDGMDV |
| light | U1-13 | 322 | RSSQSLLHSNGYNYLE | 346 | LGSNRAS | 366 | MQALQTPIT |
| heavy | U1-14 | 236 | GGSISSGDYYWS | 258 | YIYYSGSTYYNPSLKS | 283 | ADYDFWSGYFDY |
| light | U1-14 | 318 | RASQGIRNDLG | 343 | AASSLQS | 364 | LQHNTYPWT |
| heavy | U1-15 | 239 | GGSVSSGGYYWS | 261 | YIYYSGSTNYNPSLKS | 288 | DGDVDTAMVDAFDI |
| light | U1-15 | 323 | RASQSLSGNYLA | 347 | GASSRAT | 367 | QQYDRSPLT |
| heavy | U1-16 | 236 | GGSISSGDYYWS | 258 | YIYYSGSTYYNPSLKS | 283 | ADYDFWSGYFDY |
| light | U1-16 | 318 | RASQGIRNDLG | 343 | AASSLQS | 360 | LQHNSYPWT |
| heavy | U1-17 | 236 | GGSISSGDYYWS | 262 | YIYYSGSTYYNPSLKS | 283 | ADYDFWSGYFDY |
| light | U1-17 | 318 | RASQGIRNDLG | 343 | AASSLQS | 360 | LQHNSYPWT |
| heavy | U1-18 | 236 | GGSISSGDYYWS | 258 | YIYYSGSTYYNPSLKS | 283 | ADYDFWSGYFDY |
| light | U1-18 | 318 | RASQGIRNDLG | 343 | AASSLQS | 360 | LQHNSYPWT |
| heavy | U1-19 | 236 | GGSISSGDYYWS | 258 | YIYYSGSTYYNPSLKS | 289 | GDYDFWSGEFDY |
| light | U1-19 | sequence not available | | | | | |
| heavy | U1-20 | 237 | GGSISSGGYYWS | 263 | YIYDSGSTYYNPSLKS | 290 | DQGQDGYSYGYGYYYGMDV |
| light | U1-20 | 324 | QASQDISNYLN | 348 | VASNLET | 368 | QQCDNLPLT |
| heavy | U1-21 | 236 | GGSISSGDYYWS | 258 | YIYYSGSTYYNPSLKS | 283 | ADYDFWSGYFDY |
| light | U1-21 | 320 | RASQDIRNDLG | 349 | AASRLQS | 360 | LQHNSYPWT |
| heavy | U1-22 | 236 | GGSISSGDYYWS | 258 | YIYYSGSTYYNPSLKS | 283 | ADYDFWSGYFDY |
| light | U1-22 | 318 | RASQGIRNDLG | 350 | AASSLQN | 360 | LQHNSYPWT |
| heavy | U1-23 | 236 | GGSISSGDYYWS | 258 | YIYYSGSTYYNPSLKS | 283 | ADYDFWSGYFDY |
| light | U1-23 | 318 | RASQGIRNDLG | 343 | AASSLQS | 360 | LQHNSYPWT |
| heavy | U1-24 | 236 | GGSISSGDYYWS | 258 | YIYYSGSTYYNPSLKS | 285 | ADYDFWNGYFDY |
| light | U1-24 | 318 | RASQGIRNDLG | 343 | AASSLQS | 363 | LQHNNYPWT |
| heavy | U1-25 | 236 | GGSISSGDYYWS | 258 | YIYYSGSTYYNPSLKS | 283 | ADYDFWSGYFDY |
| light | U1-25 | 318 | RASQGIRNDLG | 350 | AASSLQN | 360 | LQHNSYPWT |
| heavy | U1-26 | 236 | GGSISSGDYYWS | 258 | YIYYSGSTYYNPSLKS | 291 | ADYDFWSGYFDF |
| light | U1-26 | 318 | RASQGIRNDLG | 343 | AASSLQS | 361 | LQHNGYPWT |
| heavy | U1-27 | 236 | GGSISSGDYYWS | 258 | YIYYSGSTYYNPSLKS | 291 | ADYDFWSGYFDF |
| light | U1-27 | 318 | RASQGIRNDLG | 343 | AASSLQS | 361 | LQHNGYPWT |
| heavy | U1-28 | 236 | GGSISSGDYYWS | 258 | YIYYSGSTYYNPSLKS | 292 | ADYDFWSGYFDS |
| light | U1-28 | 318 | RASQGIRNDLG | 343 | AASSLQS | 361 | LQHNGYPWT |
| heavy | U1-29 | 240 | GFTFNSYDMH | 264 | VIWYDGSNKYYADSVKG | 293 | DRLCTNGVCYEDYGMDV |
| light | U1-29 | 324 | QASQDISNYLN | 351 | DASNLET | 369 | QHYDTLPLT |
| heavy | U1-30 | 236 | GGSISSGDYYWS | 265 | YIYYSGTTYYNPSLKS | 283 | ADYDFWSGYFDY |
| light | U1-30 | 325 | RAGQGIRNDLG | 343 | AASSLQS | 360 | LQHNSYPWT |
| heavy | U1-31 | 241 | GYTFTNYGIS | 266 | WISAYDGYRNYAQKLQG | 294 | DVQDYGDYDYFDY |
| light | U1-31 | 326 | RASQSISSYLN | 343 | AASSLQS | 370 | QQSYSTPIT |
| heavy | U1-32 | 236 | GGSISSGDYYWS | 265 | YIYYSGTTYYNPSLKS | 283 | ADYDFWSGYFDY |
| light | U1-32 | 325 | RAGQGIRNDLG | 343 | AASSLQS | 360 | LQHNSYPWT |
| heavy | U1-33 | 236 | GGSISSGDYYWS | 258 | YIYYSGSTYYNPSLKS | 295 | ADYDFWSGHFDC |
| light | U1-33 | 327 | RASQGIRDDLG | 352 | AESSLQS | 371 | LQHHSYPWT |
| heavy | U1-34 | 241 | GYTFTNYGIS | 266 | WISAYDGYRNYAQKLQG | 294 | DVQDYGDYDYFDY |
| light | U1-34 | 326 | RASQSISSYLN | 343 | AASSLQS | 370 | QQSYSTPIT |
| heavy | U1-35 | 242 | GFTFSDYYMS | 267 | YISSSGNNIYHADSVKG | 296 | ERYSGYDDPDGFDI |
| light | U1-35 | 328 | QASQDISNYLS | 351 | DASNLET | 372 | QQYDNPPCS |
| heavy | U1-36 | 243 | GGSISSGYYYWS | 268 | YIYYSGTTYYNPSFKS | 297 | ADYDFWSGYFDY |
| light | U1-36 | 318 | RASQGIRNDLG | 343 | AASSLQS | 360 | LQHNSYPWT |
| heavy | U1-37 | 244 | GYTFTSYGIS | 269 | WISAYDGHTNYAQKLQG | 298 | DPHDYSNYEAFDF |
| light | U1-37 | 326 | RASQSISSYLN | 343 | AASSLQS | 370 | QQSYSTPIT |
| heavy | U1-38 | 245 | GFSLSTSGVGVG | 270 | LIYWNDDKRYSPSLKS | 299 | RDEVRGFDY |
| light | U1-38 | 329 | RSSQSLVYSDGYTYLH | 353 | KVSNWDS | 373 | MQGAHWPIT |
| heavy | U1-39 | 246 | GFTVSSNYMS | 271 | VIYSGGSTYYADSVKG | 300 | GQWLDV |
| light | U1-39 | 321 | RSSQSLLHSNGYNYLD | 354 | LGFHRAS | 374 | RQALQTPLT |
| heavy | U1-40 | 237 | GGSISSGGYYWS | 272 | YIYYSGSTYYNPSLKS | 301 | DRELELYYYYYGMDV |
| light | U1-40 | 330 | RSSQSLLYSNGYNYLD | 346 | LGSNRAS | 365 | MQALQTPLT |
| heavy | U1-41 | 237 | GGSISSGGYYWS | 258 | YIYYSGSTYYNPSLKS | 302 | DRELEGYSNYYGVDV |
| light | U1-41 | 331 | RASQAISNYLN | 343 | AASSLQS | 375 | QQNNSLPIT |
| heavy | U1-42 | 247 | GYSFTSYWIG | 273 | IIYPGDSDTRYSPSFQG | 303 | HENYGDYNY |
| light | U1-42 | 332 | RASQSIRSYLN | 343 | AASSLQS | 376 | QQSNGSPLT |
| heavy | U1-43 | 237 | GGSISSGGYYWS | 259 | YIYYSGSTYYNPSLRS | 304 | DREREWDDYGDPQGMDV |
| light | U1-43 | 333 | RASQSISSYLH | 343 | AASSLQS | 377 | QQSYSNPLT |
| heavy | U1-44 | 247 | GYSFTSYWIG | 274 | IIWPGDSDTIYSPSFQG | 303 | HENYGDYNY |
| light | U1-44 | 332 | RASQSIRSYLN | 343 | AASSLQS | 378 | QQSISSPLT |
| heavy | U1-45 | 248 | GYTFTSYDIN | 275 | WMNPNSGDTGYAQVFQG | 305 | FGDLPYDYSYYEWFDP |
| light | U1-45 | 326 | RASQSISSYLN | 343 | AASSLQS | 379 | QQSYSTPLT |
| heavy | U1-46 | 249 | GDSVSSNSAAWN | 276 | RTYYRSKWYNDYAVSVKS | 306 | DLYDFWSGYPYYYGMDV |
| light | U1-46 | sequence not available | | | | | |
| heavy | U1-47 | 249 | GDSVSSNSAAWN | 276 | RTYYRSKWYNDYAVSVKS | 307 | DYYGSGSFYYYYGMDV |
| light | U1-47 | 326 | RASQSISSYLN | 355 | AASNLQS | 380 | QQSYSTPRT |
| heavy | U1-48 | 250 | GGSISSYYWS | 277 | HIYTSGSTNYNPSLKS | 308 | EAIFGVGPYYYYGMDV |
| light | U1-48 | sequence not available | | | | | |
| heavy | U1-49 | 251 | GYTFTGYYMH | 278 | WINPNIGGTNCAQKFQG | 309 | GGRYSSSWSYYYYGMDV |
| light | U1-49 | 334 | KSSQSLLLSDGGTYLY | 356 | EVSNRFS | 381 | MQSMQLPIT |
| heavy | U1-50 | 239 | GGSVSSGGYYWS | 261 | YIYYSGSTNYNPSLKS | 310 | GGDSNYEDYYYYYGMDV |
| light | U1-50 | 335 | RASQSISIYLH | 343 | AASSLQS | 382 | QQSYTSPIT |
| heavy | U1-51 | 250 | GGSISSYYWS | 261 | YIYYSGSTNYNPSLKS | 311 | DSSYYDSSGYYLYYYAMDV |
| light | U1-51 | 319 | KSSQSVLYSSNNKNYLA | 344 | WASTRES | 383 | QQYYTTPLT |
| heavy | U1-52 | 237 | GGSISSGGYYWS | 279 | NIYYSGSTYYNPSLKS | 312 | GGTGTNYYYYYGMDV |
| light | U1-52 | 336 | RASQSVSSSYLA | 357 | GASSWAT | 384 | QQYGSSPLT |
| heavy | U1-53 | 252 | GFTFSIYSMN | 280 | YISSSSSTIYYADSVKG | 313 | DRGDFDAFDI |
| light | U1-53 | 337 | QASQDITNYLN | 351 | DASNLET | 385 | QQCENFPIT |
| heavy | U1-55.1 | 253 | GGSVSSGGYYWN | 281 | YINYSGSTNYNPSLKS | 301 | DRELELYYYYYGMDV |
| light | U1-55.1 | same as U1-55 | | | | | |
| heavy | U1-55 | same as U1-55.1 | | | | | |
| light | U1-55 | 338 | RSSQSLLYSNGYKYLD | 346 | LGSNRAS | 366 | MQALQTPIT |
| heavy | U1-57.1 | same as U1-57 | | | | | |
| light | U1-57.1 | 338 | RSSQSLLYSNGYKYLD | 346 | LGSNRAS | 366 | MQALQTPIT |
| heavy | U1-57 | 254 | GGSVSSGGYYWN | 281 | YINYSGSTNYNPSLKS | 301 | DRELELYYYYYGMDV |
| light | U1-57 | same as U1-57.1 | | | | | |
| heavy | U1-58 | 255 | GFTFSSYGMH | 264 | VIWYDGSNKYYADSVKG | 314 | AARLDYYYGMDV |
| light | U1-58 | 339 | RASQSINSYLN | 358 | GASGLQS | 386 | QQSYSSPLT |
| heavy | U1-59 | 256 | GGSFSGYYWS | 282 | EINHSGSTNYNPSLKS | 315 | DKWTWYFDL |
| light | U1-59 | 340 | RSSQSVLYSSSNRNYLA | 344 | WASTRES | 387 | QQYYSTPRT |
| heavy | U1-61.1 | 257 | GVSISSGGYYWS | 258 | YIYYSGSTYYNPSLKS | 316 | DSESEYSSSSNYGMDV |
| light | U1-61.1 | same as U1-61.1 | | | | | |
| heavy | U1-61 | 257 | GVSISSGGYYWS | 258 | YIYYSGSTYYNPSLKS | 316 | DSESEYSSSSNYGMDV |
| light | U1-61 | 341 | RASQTISSYLN | 359 | AASSLQG | 377 | QQSYSNPLT |
| heavy | U1-62 | 247 | GYSFTSYWIG | 273 | IIYPGDSDTRYSPSFQG | 317 | QMAGNYYYGMDV |
| light | U1-62 | 342 | RASQSVISIYLA | 347 | GASSRAT | 388 | QQYGSSPCS |

In some embodiments, an isolated binding protein targeted to HER3 can include a heavy chain amino acid sequence selected from the group consisting of SEQ ID NOS: 2, 6, 10, 14, 18, 22, 26, 30, 34, 36, 40, 42, 46, 50, 54, 60, 62, 66, 70, 74, 78, 80, 84, 88, 92, 96, 100, 104, 108, 112, 116, 120, 122, 126, 130, 134, 138, 142, 146, 150, 154, 158, 162, 166, 170, 174, 178, 182, 186, 190, 194, 198, 202, 206, 210, 214, 218, 222, 226 and 230, and/or a light chain amino acid sequence selected from the group consisting of SEQ ID NOS: 4, 8, 12, 16, 20, 24, 28, 32, 38, 44, 48, 52, 56, 58, 64, 68, 72, 76, 82, 86, 90, 94, 98, 102, 106, 110, 114, 118, 124, 128, 132, 136, 140, 144, 148, 152, 156, 160, 164, 168, 172, 176, 180, 184, 188, 192, 196, 200, 204, 208, 212, 216, 220, 224, 228 and 232. The sequences referred to are shown in the sequence listing in U.S. Pat. No. 7,705,130, and are hereby incorporated herein by reference in their entireties.

In some embodiments, an anti-HER3 antibody can include a heavy chain amino acid sequence and a light chain amino acid sequence as shown in SEQ ID NOS: 2 and 4, 6 and 8, 10 and 12, 14 and 16, 18 and 20, 22 and 24, 26 and 28, 30 and 32, 36 and 38, 42 and 44, 46 and 48, 50 and 52, 54 and 56, 60 and 58, 62 and 64, 66 and 68, 70 and 72, 74 and 76, 78 and 82, 80 and 82, 84 and 86, 88 and 90, 92 and 94, 96 and 98, 100 and 102, 104 and 106, 108 and 110, 112 and 114, 116 and 118, 122 and 124, 126 and 128, 130 and 132, 134 and 136, 138 and 140, 142 and 144, 146 and 148, 150 and 152, 154 and 156, 158 and 160, 162 and 164, 166 and 168, 170 and 172, 174 and 176, 178 and 180, 182 and 184, 186 and 188, 190 and 192, 194 and 196, 198 and 200, 202 and 204, 206 and 208, 210 and 212, 214 and 216, 218 and 220, 222 and 224, 226 and 228, 230 and 232, or a heavy chain amino acid sequence as shown in any one of SEQ ID NOS: 34, 40, 60, 62, and 120, or a light chain amino acid sequence as shown in either of SEQ ID NOS: 58 and 64. The sequences referred to appear in the disclosure and the sequence listing of U.S. Pat. No. 7,705,130, and are hereby incorporated herein by reference in their entireties.

In some embodiments, a protein targeted to HER3 can be a scaffold protein having an antibody-like binding activity (e.g., having activity similar to an anti-HER3 antibody), or an antibody, e.g., an anti-HER3 antibody. The anti-HER3 antibody can be selected from the group consisting of antibodies designated U1-1, U1-2, U1-3, U1-4, U1-5, U1-6, U1-7, U1-8, U1-9, U1-10, U1-11, U1-12, U1-13, U1-14, U1-15, U1-16, U1-17, U1-18, U1-19, U1-20, U1-21, U1-22, U1-23, U1-24, U1-25, U1-26, U1-27, U1-28, U1-29, U1-30, U1-31, U1-32, U1-33, U1-34, U1-35, U1-36, U1-37, U1-38, U1-39, U1-40, U1-41, U1-42, U1-43, U1-44, U1-45, U1-46, U1-47, U1-48, U1-49, U1-50, U1-51, U1-52, U1-53, U1-55.1, U1-55, U1-57.1, U1-57, U1-58, U1-59, U1-61.1, U1-61, and U1-62, or an antibody having at least one heavy or light chain of one of the aforeto antibodies. The antibodies designated as U1-49 (SEQ ID NO: 42/44), U1-53 (SEQ ID NO: 54/56), and U1-59 (SEQ ID NO: 70/72), or an antibody having at least one heavy or light chain of one of these antibodies, may be particularly useful. All sequences of the antibodies and sequences thereof referred to are shown in the sequence listing and disclosure of U.S. Pat. No. 7,705,130, and are hereby incorporated herein by reference in their entireties.

It is to be understood that the amino acid sequence of the HER3 binding proteins provided herein is not limited to the twenty conventional amino acids (See: Immunology—A Synthesis, 2^{nd} Edition, Golub and Gren, eds., Sinauer Associates, Sunderland, Mass. (1991)), which publication is hereby incorporated herein by reference in its entirety. For example, the amino acids may include stereoisomers (e.g., D-amino acids) of the twenty conventional amino acids, unnatural amino acids such as I-,I-disubstituted amino acids, N-alkyl amino acids, lactic acid, and other unconventional amino acids. Examples of unconventional amino acids, which may also be suitable components for the binding proteins provided herein, include: 4-hydroxyproline, K-carboxyglutamate, M-N,N,N-trimethyllysine, M-N-acetyllysine, O-phosphoserine, N-acetylserine, N-formylmethionine, 3-methylhistidine, 5-hydroxylysine, σ-N-methylarginine, and other similar amino acids and imino acids, e.g., 4-hydroxyproline.

Furthermore, minor variations in the amino acid sequences shown in SEQ ID NOS: 1-390 (as set forth in the appendix filed herewith) are contemplated as being encompassed by the present disclosure, provided that the variations in the amino acid sequence maintain at least 75% (e.g., at least 80%, 90%, 95%, or 99%) of the sequences shown in SEQ ID NOS: 1-390. Variations can occur within the framework regions (i.e., outside the CDRs), within the CDRs, or within the framework regions and the CDRs. In some embodiments, variations in the amino acid sequences shown in SEQ ID NOS: 1-390, i.e., deletions, insertions and/or substitutions of at least one amino acid, can occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other binding proteins of known structure and/or function. Methods for identifying protein sequences that fold into a known three-dimensional structure are known in the art. (See: Bowie et al., Science 253:164 (1991); Proteins, Structures and Molecular Principles, Creighton, Ed., W.H. Freeman and Company, New York (1984); Introduction to Protein Structure, Branden and Tooze, eds., Garland Publishing, New York, N.Y. (1991); and Thornton et al. Nature 354:105 (1991), which publications are hereby incorporated herein by reference in their entireties. Thus, those of skill in the art can recognize sequence motifs and structural conformations that may be used to define structural and functional domains in accordance with the proteins described herein.

Variations in the amino acid sequences shown in SEQ ID NOS:1-390 can include those that lead to a reduced susceptibility to proteolysis or oxidation, alter glycosylation patterns or alter binding affinities or confer or modify other physicochemical or functional properties of the binding protein. In particular, conservative amino acid replacements are contemplated. Conservative replacements are those that take place within a family of amino acids that are related in their side chains. Amino acid families include the following: acidic family=aspartate, glutamate; basic family=lysine, arginine, histidine; non-polar family=alanine, valine, leucine, isoleucine, proline, phenylalanine, methionine, tryptophan; and uncharged polar family=glycine, asparagine, glutamine, cysteine, serine, threonine, tyrosine. Alternative families include: aliphatic-hydroxy family=serine and threonine; amide-containing family=asparagine and glutamine; aliphatic family=alanine, valine, leucine and isoleucine; and aromatic family=phenylalanine, tryptophan, and tyrosine. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting binding protein, especially if the replacement does not involve an amino acid within a framework site. However, all other possible amino acid replacements also are encompassed herein. Whether an amino acid change results in a functional HER3 binding protein that reduces signal transduction of HER3 can readily be determined by assaying the specific HER3 binding activity of the resulting binding protein by ELISA or FACS, or in vitro or in vivo functional assays.

In some embodiments, a HER3 binding protein can be coupled to an effector group. Such a binding protein can be especially useful for therapeutic applications. As used herein, the term “effector group” refers to a cytotoxic group such as a radioisotope or radionuclide, a toxin, a therapeutic group or other effector group known in the art. Examples of suitable effector groups are radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I) or non-radio isotopes (e.g., 2D), calicheamicin, dolastatin analogs such as auristatins, and chemotherapeutic agents such as geldanamycin and maytansine derivates, including DM1. Thus, in some cases, a group can be both a labeling group and an effector group. For example, this includes Toxins, RNA Polymerase Inhibitors or other compounds suitable for use as ADC (Antibody Drug conjugate). Various methods of attaching effector groups to polypeptides or glycopolypeptides (such as antibodies) are known in the art, and may be used in making and carrying out the compositions and methods described herein. In some embodiments, it may be useful to have effector groups attached to a binding protein by spacer arms of various lengths to, for example, reduce potential steric hindrance.

This document also relates to processes for preparing an isolated HER3 binding protein, comprising the step of preparing the protein from a host cell that expresses the protein. Host cells that can be used include, without limitation, hybridomas, eukaryotic cells (e.g., mammalian cells such as hamster, rabbit, rat, pig, or mouse cells), plant cells, fungal cells, yeast cells (e.g., Saccharomyces cerevisiae or Pichia pastoris cells), prokaryotic cells (e.g., E. coli cells), and other cells used for production of binding proteins. Various methods for preparing and isolating binding proteins, such as scaffold proteins or antibodies, from host cells are known in the art and may be used in performing the methods described herein. Moreover, methods for preparing binding protein fragments, e.g., scaffold protein fragments or antibody fragments, such as papain or pepsin digestion, modern cloning techniques, techniques for preparing single chain antibody molecules (Plückthun, supra) and diabodies (Hollinger et al., supra), also are known to those skilled in the art and may be used in performing the presently described methods.

In some embodiments, a HER3 binding protein can be prepared from a hybridoma that secretes the protein. See: Köhler et al. Nature 256:495 (1975).

In some embodiments, a HER3 binding protein can be prepared recombinantly by optimizing and/or amplifying expression of the binding protein in host cells, and isolating the binding protein from the host cells. To this end, host cells can be transformed or transfected with DNA (e.g., a vector) encoding a HER3 binding protein, and cultured under conditions appropriate to produce the binding protein. See: U.S. Pat. No. 4,816,567. Useful host cells include, for example, CHO cells, NS/0 myeloma cells, human embryonic kidney 293 cells, E. coli cells, and Saccharomyces cerevisiae cells.

HER3 binding proteins that are antibodies can be prepared from animals genetically engineered to make fully human antibodies, or from an antibody display library made in bacteriophage, yeast, ribosome or E. coli. See: Clackson et al., Nature 352:624-628 (1991); Marks et al., J. Mol. Biol. 222:581-597 (1991); Feldhaus and Siegel, J. Immunol. Methods 290:69-80 (2004); Groves and Osbourn, Expert Opin. Biol. Ther. 5:125-135 (2005); and Jostock and Dubel, Comb. Chem. High Throughput Screen 8:127-133 (2005), which publications are hereby incorporated herein by reference in their entireties.

In some embodiments, antibodies as provided herein can be fully human or humanized antibodies. Human antibodies avoid certain problems associated with xenogeneic antibodies, such as antibodies that possess murine or rat variable and/or constant regions. The presence of xenogeneic-derived proteins can lead to an immune response against the antibody by a patient, subsequently leading to the rapid clearance of the antibody, loss of therapeutic utility through neutralization of the antibody, and/or severe, even life-threatening, allergic reactions. To avoid using murine or rat-derived antibodies, fully human antibodies can be generated through the introduction of functional human antibody loci into a rodent or another mammal or animal so that the rodent, other mammal or animal produces fully human antibodies.

One method for generating fully human antibodies is to utilize XENOMOUSE® strains of mice that have been engineered to contain 245 kb and 190 kb-sized germline configuration fragments of the human heavy chain locus and kappa light chain locus. Other XENOMOUSE® strains of mice contain 980 kb and 800 kb-sized germline configuration fragments of the human heavy chain locus and kappa light chain locus. Still other XENOMOUSE® strains of mice contain 980 kb and 800 kb-sized germline configuration fragments of the human heavy chain locus and kappa light chain locus plus a 740 kb-sized germline configured complete human lambda light chain locus. See: Mendez et al., Nature Genetics 15:146-156 (1997); and Green and Jakobovits, J. Exp. Med. 188:483-495 (1998). XENOMOUSE® strains are available from Amgen, Thousand Oaks, Calif.

The production of XENOMOUSE® mice is further discussed and delineated in: US Patent Publication No. 20030217373, filed Nov. 20, 2002; U.S. Pat. Nos. 5,939,598, 6,075,181, 6,114,598, 6,150,584, 6,162,963, 6,673,986, 6,833,268, and 7435871; and Japanese Patent Nos. 3068180B2, 3068506B2, and 3068507B2. Also See: Patent No. EP 0463151; and PCT Publication Nos. WO94/02602, WO96/34096, WO98/24893, and WO00/76310. Each of the disclosures of the above publications and patents is hereby incorporated herein by reference in it's entirety.

Alternatively, a “minilocus” approach can be used. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (e.g., a gamma constant region) are formed into a construct for insertion into an animal. This approach is described in U.S. Pat. Nos. 5,545,806, 5,545,807, 5,569,825, 5,591,669, 5,612,205, 5,625,126, 5,625,825, 5,633,425, 5,643,763, 5,661,016, 5,721,367, 5,770,429, 5,789,215, 5,789,650, 5,814,318, 5,874,299, 5,877,397, 5,981,175, 6,023,010, 6,255,458, the disclosures of which are hereby incorporated herein by reference in their entirety. Also See: Patent No. EP0546073; PCT Publication Nos. WO92/03918, WO92/22645, WO92/22647, WO92/22670, WO93/12227, WO94/00569, WO94/25585, WO96/14436, WO97/13852, and WO98/24884, the disclosures of which are hereby incorporated herein by reference in their entireties.

Human antibodies also can be generated from mice in which, through microcell fusion, large pieces of chromosomes, or entire chromosomes, have been introduced. See: Patent Application Nos. EP773288 and EP843961, the disclosures of which are hereby incorporated herein by reference in their entireties. Additionally, KM™ mice, which are the result of cross-breeding of Kirin's Tc mice with Medarex's minilocus (Humab) mice have been generated. These mice possess the HC transchromosome of the Kirin mice and the kappa chain transgene of the Medarex mice (See: Ishida et al., Cloning Stem Cells 4:91-102 (2002)).

Human antibodies also can be derived by in vitro methods. Suitable examples include, but are not limited to, phage display (as commercialized by Cambridge Antibody Technology, Morphosys, Dyax, Biosite/Medarex, Xoma, Symphogen, Alexion (formerly Proliferon), and Affimed), ribosome display (as commercialized by Cambridge Antibody Technology), yeast display, and the like.

As described herein, antibodies were prepared using XENOMOUSE® technology, as described below. Such mice are capable of producing human immunoglobulin molecules and antibodies, and are deficient in the production of murine immunoglobulin molecules and antibodies. Technologies utilized for achieving the same are disclosed in the patents, applications, and references disclosed herein. For example, transgenic production of mice and antibodies therefrom is disclosed in US Patent Application No. 08759620, filed Dec. 3, 1996, now abandoned, PCT Publication Nos. WO98/24893, published Jun. 11, 1998 and WO00/76310, published Dec. 21, 2000, the disclosures of which are hereby incorporated herein by reference in their entirety. Also See: Mendez et al., Nature Genetics, 15:146-156 (1997), the disclosure of which is hereby incorporated herein by reference in its entirety.

Using technology as described herein, fully human monoclonal antibodies to a variety of antigens can be produced. For example, XENOMOUSE® lines of mice can be immunized with a HER3 antigen of interest (e.g., HER3 or a fragment thereof), lymphatic cells (such as B-cells) can be recovered from mice that express antibodies, and the recovered cell lines can be fused with a myeloid-type cell line to prepare immortal hybridoma cell lines. These hybridoma cell lines can be screened and selected to identify hybridoma cell lines that produce antibodies specific to the antigen of interest. Provided herein are methods for the production of multiple hybridoma cell lines that produce antibodies specific to HER3. Further provided herein are methods for characterizing antibodies produced by such cell lines, including nucleotide and amino acid sequence analyses of the heavy and light chains of such antibodies.

In general, antibodies produced by fused hybridomas as described below are human IgG1 heavy chains with fully human kappa light chains, although some antibodies described herein possess human IgG4 heavy chains as well as IgG1 heavy chains. Antibodies also can be of other human isotypes, including IgG2 and IgG3. The antibodies generally have high affinities, with a K_{D} typically from about 10⁻⁶ to about 10⁻¹³ M or below, when measured by solid phase and cell-based techniques.

This document also provides isolated nucleic acid molecules that encode HER3 binding proteins as described herein. The term “isolated nucleic acid molecule,” as used herein, refers to a polynucleotide of genomic, cDNA, or synthetic origin, or some combination thereof, which (1) is not associated with all or a portion of a polynucleotide with which the “isolated polynucleotide” is found in nature, (2) is operably linked to a polynucleotide to which it is not linked to in nature, or (3) does not occur in nature as part of a larger sequence. Further, the term “nucleic acid molecule,” as used herein, means a polymeric form of nucleotides of at least 10 bases in length, either ribonucleotides or deoxynucleotides or a modified form of either type of nucleotide, such as nucleotides with modified or substituted sugar groups and the like. The term also includes single and double stranded forms of DNA.

In some embodiments, a nucleic acid molecule can be operably linked to a control sequence. The term “control sequence,” as used herein, refers to polynucleotide sequences that are necessary to effect the expression and processing of coding sequences to which they are ligated. The nature of such control sequences differs depending upon the host organism. In prokaryotes, such control sequences generally include promoters, ribosomal binding sites, and transcription termination sequences. In eukaryotes, generally, such control sequences include promoters and transcription termination sequences. The term “control sequence” is intended to include, at a minimum, all components whose presence is essential for expression and processing, and can also include additional components whose presence is advantageous, for example, leader sequences and fusion partner sequences. Furthermore, the term “operably linked”, as used herein, refers to positions of components so described which are in a relationship permitting them to function in their intended manner. Moreover, an expression control sequence operably linked to a coding sequence is ligated in such a way that expression of the coding sequence is achieved under conditions compatible with the expression control sequence.

Also provided herein are vectors comprising a nucleic acid molecule encoding a binding protein as disclosed herein. The nucleic acid molecule can be operably linked to a control sequence. Furthermore, the vector may additionally contain a replication origin or a selection marker gene. Examples of vectors that may be used include, e.g., plasmids, cosmids, phages, and viruses.

This document also provides host cells transformed with a nucleic acid molecule or vector as described herein. Transformation can be accomplished by any known method for introducing polynucleotides into a host cell, including, for example, packaging the polynucleotide in a virus (or into a viral vector) and transducing a host cell with the virus (or vector), or by transfection procedures known in the art, as exemplified by U.S. Pat. Nos. 4,399,216, 4,912,040, 4,740,461, and 4959455. Methods for introducing heterologous polynucleotides into mammalian cells are well known in the art, and include, without limitation, dextran-mediated transfection, calcium phosphate precipitation, polybrene mediated transfection, protoplast fusion, electroporation, encapsulation of the polynucleotide(s) in liposomes, and direct microinjection of the DNA into nuclei. Examples of host cells that may be used include hybridomas, eukaryotic cells (e.g., mammalian cells such as hamster, rabbit, rat, pig, mouse, or other animal cells), plant cells (e.g., corn and tobacco cells), fungal cells (e.g., S. cerevisiae and P. pastoris cells), prokaryotic cells such as E. coli, and other cells used in the art for production of antibodies. Mammalian cell lines available as hosts for expression are well known in the art and include, for example, many immortalized cell lines available from the American Type Culture Collection (ATCC; Manassas, Va.). These include, without limitation, Chinese hamster ovary (CHO) cells, HeLa cells, baby hamster kidney (BHK) cells, monkey kidney cells (COS), human hepatocellular carcinoma cells (e.g., Hep G2 cells), and a number of other cell lines.

In other embodiments, the agent binding to HER3 is a small molecule compound. Such compounds can be identified using, for example, physical or virtual libraries of small molecules. In some embodiments, for example, useful small molecule compounds can be identified using consensus virtual screening methods based on known HER3 inhibitors and models of HER3 active and inactive state structures. Compounds that appear to be of interest can be further analyzed for structural novelty and desirable physicochemical properties. Candidate compounds identified by virtual screening can be tested in vitro for, e.g., the ability to inhibit growth of cells that overexpress HER3. In other embodiments, useful small molecule compounds can be identified from a library of small molecule compounds, using high throughput methods to screen large numbers of compounds for the ability to bind to and/or inhibit activity of HER3 (e.g., in cells that overexpress HER3). Small molecule HER3 inhibitors can be synthesized using standard chemical synthesis methods, for example.

In yet another embodiment, the agent that binds to HER3 may be a siRNA that interferes with the expression of HER3. An example of siRNA is EZN-3920 (antisense targeting HER3 mRNA) (Santaris Pharma, Hoersholm, Denmark).

In yet other embodiments, the agent that binds HER3 may be a natural substance. For example, Kahalalide F, a marine-derived agent, has been suggested to inhibit HER3 oncogenic signaling (See: Jimeno et al., J. Translational Med. 4:3 (2006)) by down-regulating HER3 protein expression and AKT signaling (See: Janmaat et al., Mol. Pharmacol. 68:502-510 (2005)).

### 4. Additional Agents

In some cases, the compositions and methods provided herein for prostate treatment can include a first agent that binds to HER3 in combination with a second agent. In some cases, the second agent can bind and/or inhibit at least one other member of the HER family, such as EGF-R, HER2, or HER4. Such a second agent can be, without limitation, a biological drug (e.g., a binding protein, such as an antibody that specifically binds to a member of the HER family, a small molecular compound that binds to and/or alters (e.g., inhibits) the activity of at least one member of the HER family other than (or in addition to) HER3, an siRNA, or a natural substance. As used herein, the terms “other HER family members” and “another HER family member” refer to HER family members that are not HER3. Examples include EGF-R, HER2, and HER4, but “HER family member” also includes family members that have not yet been identified.

The second agent can alter the activity (e.g., increase or decrease the activity) of the other HER family member, either through a direct effect or an indirect effect on the HER family member. It is noted, however, that all second agents as provided herein will have an effect on HER family function and activity. In some cases, for example, the second agent can be an antibody that can bind to another HER family member (e.g., EGF-R, HER2, or HER4), or to another molecule that in turn can affect the activity of the other HER family member. Such an antibody can be targeted, for example, to the extracellular domain of the other HER family member, or to any other suitable domain thereof (e.g., a kinase domain or a dimerization domain).

A second agent can be further characterized in that its effect on another HER family member reduces HER-mediated signal transduction. A reduction of HER-mediated signal transduction may, e.g., be caused by downregulation of the targeted HER family member, resulting in an at least partial disappearance of the HER molecule from the cell, or by a stabilization of the HER family member in a substantially inactive form. Alternatively, a reduction of HER-mediated signal transduction may be caused by influencing, e.g., decreasing or inhibiting, the binding of a ligand to the HER family member, the binding of the HER family member to HER3, or the binding of GRB2 to HER2 or GRB2 to SHC, or, by inhibiting receptor tyrosine phosphorylation, AKT phosphorylation, PYK2 tyrosine phosphorylation, or ERK2 phosphorylation, or any other cellular component affecting the HER-family mediated signal transduction pathway. For example, a reduction of HER mediated signal transduction can be caused by decreasing the formation of dimers containing HER3 and another HER family member (e.g., EGF-R, HER2, or HER4). Regardless of the mechanism behind the function, it is noted that the second agent can serve to amplify the effect of the first agent that is targeted to HER3.

In some embodiments, an agent that binds to another HER family member or another protein that in turn affects activity of another HER family member can be a scaffold protein having an antibody like binding activity (e.g., having activity similar to an anti-HER3 antibody) or an antibody (e.g., an anti-EGF-R, anti-HER2, or anti-HER4 antibody). Scaffold proteins and antibodies in this context are as defined and described above for agents targeted to HER3.

It is noted that in some embodiments, the first agent that binds to HER3, and the second agent that binds to and/or inhibits another HER family member are combined within one compound, such as a bispecific antibody.

Also as described above, the amino acid sequences of proteins that bind to other HER family members, or to other proteins that in turn affect the activity of another HER family member, are not limited to the twenty conventional amino acids. Further, as for the HER3 binding proteins described herein, an agent that binds to or otherwise affects the activity of another HER family member can be coupled to an effector group.

This document also relates to processes for preparing isolated proteins (e.g., antibodies) that can bind to other HER family members, for example. Such processes include those described above in the context of HER3 binding proteins. In some embodiments, antibodies (e.g., anti-HER, anti-HER2, or anti-HER4 antibodies, respectively) can be prepared from animals engineered to make fully human antibodies, or from an antibody display library made in bacteriophage, yeast, ribosomes, or E. coli. Further, an antibody targeted directly or indirectly to another HER family member can be fully human or humanized, as described above.

Also provided herein are isolated nucleic acid molecules (e.g., vectors) expressing proteins that can bind to other HER family members and other proteins that can affect the activity of other HER family members. Protein coding sequences within such nucleic acid molecules can be operably linked to one or more control sequences, as described above. Further, nucleic acid molecules can be transformed or transfected into a host cell as described above.

In some embodiments, the second agent is a small molecular tyrosine kinase inhibitor provided that the agent can affect (either directly or indirectly) the activity of a HER family member other than (or in addition to) HER3. Such inhibitors can be identified using, for example, physical or virtual libraries of small molecules. In some embodiments, for example, useful small molecule compounds can be identified using consensus virtual screening methods based on known tyrosine kinase inhibitors and models of HER family member structures in active and inactive states. Compounds that are initially identified as being of potential interest can be further analyzed for structural novelty and desirable physicochemical properties. Candidate compounds identified by virtual screening can be tested in vitro for, e.g., the ability to inhibit growth of cells that overexpress a HER family member other than HER3. In other embodiments, useful small molecule tyrosine kinase inhibitors can be identified from a library of small molecule compounds and using high throughput methods to screen large numbers of the compounds for the ability to bind to and/or inhibit activity of one or more HER family members other than HER3 (e.g., in cells that overexpress the HER protein). Small molecular tyrosine kinase inhibitors can be synthesized using, for example, standard chemical synthesis methods.

Agents that can affect an activity of EGF-R (HER) include AEE-788 (Novartis, Basel, Switzerland), BIBW-2992(N-[4-(3-chloro-4-fluorophenyl)amino]-7-[[(3S)-tetrahydro-3-furanyl]oxy]-6-quinazolinyl]-4-(dimethylamino)-2-butenamide (Boehringer Ingelheim, Ingelheim, Germany), BMS-599626 (Bristol-Myers Squibb, New York, N.Y.), BMS-690514 (Bristol-Myers Squibb, New York, N.Y.), carnetinib dihydrochloride (N-[4-[N-(3-chloro-4-fluorophenyl)amino]-7-[3-(4-morpholinyl)propoxy]quinazolin-6-yl]acrylamide dihydrochloride (Pfizer, New York, N.Y.), CNX-222 (Avila Therapeutics, Waltham, Mass.), CUDC-101 (Curis, U.S. Pat. No. 7,547,781), Dimercept (Receptor Biologix, Palo Alto, Calif.), lapatinib (ditosilate hydrate (N-[3-chloro-4-[(3-fluorobenzyl)oxy]phenyl]-6-[5-[[[2-(methylsulfonyl)ethyl]amino]methyl]furan-2-yl]quinazolin-4-amine bis(4-methylbenzenesulfonate)monohydrate (GlaxoSmithKline, London, England), MP-412 (Mitsubishi Tanabe Pharma Co., Osaka, Japan), neratinib ((2E)-N-4-[3-chloro-4-[(pyridin-2-yl)methoxy]phenyl]]-3-cyano-7-ethoxyquinolin-6-yl]-4-(dimethylamino)but-2-enamide) (Wyeth, Madison, N.J.), S-222611 (Shionogi, Osaka, Japan), varlitinib (4-N-[3-chloro-4-(thiazol-2-ylmethoxy)phenyl]-6-N-[(4R)-4-methyl-4,5-dihydrooxazol-2-yl]quinazoline-4,6-diamine bis(4-methylbenzenesulfonate) (Array BioPharma, Boulder, Colo.), AGT-2000 (ArmeGen Technologies, Santa Monica, Calif.), AZD-4769 (AstraZeneca, London, England), BIBX-1382 (Boehringer Ingelheim, Ingelheim, Germany), CGP-52411 (4,5-bis(phenylamino)-1H-isoindole-1,3(2H)-dione) (Novartis, Basel, Switzerland), CL-387785 (N-[4-[(3-bromophenyl)amino]-6-quinazolinyl]-2-butynamide) (Wyeth, Madison, N.J.), CP-292597 (Pfizer, New York, N.Y.), DAB-1059 (Mitsubishi Tanabe Pharma Co., Osaka, Japan), erlotinib (hydrochloride(4-(3-ethynylphenylamino)-6,7-bis(2-methoxyethoxy)-quinazoline hydrochloride (OSI Pharmeceuticals, Long Island, N.Y., U.S. Pat. No. 5,747,498), gefitinib(4-(3-chloro-4-fluorophenylamino)-7-methoxy-6-[3-(4-morpholinyl)propoxy]quinazoline) (AstraZeneca, London, England, U.S. Pat. No. 5,821,246), HMPL-813 (Hutchison China MediTech, Hong Kong), MDP-01, (Med Discovery, Plan-Les-Ouates, Switzerland), MT-062 (Medisyn Technologies, Minneapolis, Minn.), ONC-101 (Oncalis, Schlieren, Switzerland), PD-153035 (4-(3-bromophenylamino)-6,7-dimethoxyquinazoline) (AstraZeneca, London, England), PD-169540 (Pfizer, New York, N.Y.), pelitinib (Wyeth Pharmaceuticals, Madison, N.J.), PF-299804 (Pfizer, New York, N.Y.), PKI-166 (4-(R)-phenethylamino-6-(hydroxyl)phenyl-7H-pyrrolo[2,3-d]-pyrimidine) (Novartis, Basel, Switzerland), vandetanib (N-(4-bromo-2-fluorophenyl)-6-methoxy-7-[(1-methylpiperidin-4-yl)methoxy]quinazolin-4-amine) (AstraZeneca, London, England), VGA-1102 (Taisho Pharmaceuticals, Tokyo, Japan), WHI-P154 (4-(3′-bromo-4′-hydroxyphenyl)-amino-6,7-dimethoxyquinazoline), ZD-1838 (AstraZeneca, London, England), cetuximab (ImClone Systems, New York, N.Y.), panitumumab (Amgen, Thousand Oaks, Calif.).

Agents that can affect an activity of HER2 include AEE-788 (Novartis, Basel, Switzerland), ARRY-333786 (Array BioPharma, Boulder, Colo.), ARRY-380 (Array BioPharma, Boulder, Colo.), BIBW-2992 (N-[4-(3-chloro-4-fluorophenyl)amino]-7-[[(3S)-tetrahydro-3-furanyl]oxy]-6-quinazolinyl]-4-(dimethylamino)-2-butenamide (Boehringer Ingelheim, Ingelheim, Germany), BMS-599626 (Bristol-Myers Squibb, New York, N.Y.), BMS-690514 (Bristol-Myers Squibb, New York, N.Y.), carnetinib dihydrochloride (N-[4-[N-(3-chloro-4-fluorophenyl)amino]-7-[3-(4-morpholinyl)propoxy]quinazolin-6-yl]acrylamide dihydrochloride) (Pfizer, New York, N.Y.), CNF-201 (Biogen Idec, San Diego, Calif.), CNX-222 (Avila Therapeutics, Waltham, Mass.), CP-654577 (OSI Pharmaceuticals, Long Island, N.Y.), CP-724714 (2-methoxy-N-[3-[4-[3-methyl-4-(6-methyl-pyridin-3-yloxy)phenylamino]quinazolin-6-yl]-E-allyl]acetamide) (OSI Pharmaceuticals, Long Island, N.Y.), CUDC-101 (Curis, Cambridge, Mass., U.S. Pat. No. 7,547,781), D-69491 (Baxter International, Deerfield, Ill.), Dimercept (Receptor Biologix, Palo Alto, Calif.), EHT-102 (ExonHit Therapeutics, Paris, France), HER2 antagonist (Centgent Therapeutics, San Diego, Calif.), HER/neu vaccine (Corixa, Seattle, Wash.), Herzyme (Sirna Therapeutics, San Francisco, Calif.), HuMax-Her2 (Genmab, Copenhagen, Denmark), INSM-18 (Insmed, Richmond, Va.), lapatinib (ditosilate hydrate(N-[3-chloro-4-[(3-fluorobenzyl)oxy]phenyl]-6-[5-E2-(methylsulfonyl)ethyl]amino]methyl]furan-2-yl]quinazolin-4-amine bis(4-methylbenzenesulfonate)monohydrate) (GlaxoSmithKline, London, England), MP-412 (Mitsubishi Tanabe Pharma Co., Osaka, Japan), mu-4-D-5 (Genentech, South San Francisco, Calif.), mubritinib (1-[4-[4-[[2-[(E)-2-[4-(trifluoromethyl)phenyl]ethenyl]oxazol-4-yl]methoxy]phenyl]butyl]-1H-1,2,3-triazole) (Takeda Pharmaceuticals, Deerfield, Ill.), neratinib ((2E)-N-[4-[[3-chloro-4-[(pyridin-2-yl)methoxy]phenyl]]-3-cyano-7-ethoxyquinolin-6-yl]-4-(dimethylamino)but-2-enamide): (Wyeth, Madison, N.J.), pertuzumab (Genentech, South San Francisco, Calif.), PX-103.1 (Pharmexa, Copenhagen, Denmark), PX-103.2 (Pharmexa, Copenhagen, Denmark), PX-104.1 (Pharmexa, Copenhagen, Denmark), S-222611 (Shionogi, Osaka, Japan), TAK-285 (Takeda Pharmaceuticals, Deerfield, Ill.), trastuzumab (Genentech, South San Francisco, Calif.), Trastuzumab-DM1 (ImmunoGen, Waltham, Mass.), varlitinib (4-N-[3-chloro-4-(thiazol-2-ylmethoxy)phenyl]-6-N-[(4R)-4-methyl-4,5-dihydrooxazol-2-yl]quinazoline-4,6-diamine bis(4-methylbenzenesulfonate)) (Array BioPharma, Boulder, Colo.), VM-206 (ViroMed, Minneapolis, Minn.).

Agents that can affect an activity of HER4 include Dimercept (Receptor Biologix, Palo Alto, Calif.), neratinib ((2E)-N-[4-[[3-chloro-4-[(pyridin-2-yl)methoxy]phenyl]amino]-3-cyano-7-ethoxyquinolin-6-yl]-4-(dimethylamino)but-2-enamide) (Wyeth, Madison, N.J.).

Particular non-limiting examples of agents that can bind to and/or alter activity of other HER family members and can be used in the compositions and methods provided herein are described below.

Panitumumab, marketed as VECTIBIX® (Amgen, Thousand Oaks, Calif.), is a fully human monoclonal antibody specific to EGF-R.

Erlotinib, marketed as TARCEVA™ (Genentech, South San Francisco, Calif.; OSI Pharmaceuticals, Long Island, N.Y.; Roche, Basel, Switzerland), is a drug used to treat NSCLC, pancreatic cancer, and several other types of cancer. Erlotinib specifically targets the EGF-R tyrosine kinase, binding reversibly to the ATP binding site of the receptor.

Lapatinib (GlaxoSmithKline, London) is an orally active small molecule for the treatment of solid tumors such as breast cancer. Lapatinib is a dual tyrosine kinase inhibitor that inhibits tyrosine kinase activity associated with EGF-R and HER2/neu (human EGF-R type 2).

Trastuzumab, also known as HERCEPTIN® (Genentech, South San Francisco, Calif.), is a humanized monoclonal antibody that interferes with the HER2/neu receptor.

T-DM1 (Genentech, South San Francisco, Calif.; Roche) is an antibody-drug conjugate that includes trastuzumab chemically linked to a potent antimicrotubule drug (DM1) derived from maytansine. Maytansine has been used as a free drug, and has shown effectiveness in, e.g., breast and lung cancer patients. The non-reducible thioether MCC linker is used in T-DM1, providing a stable bond between trastuzumab and DM1, prolonging exposure, and reducing the toxicity of T-DM1 while maintaining activity.

Pertuzumab, marketed as OMNITARG™ (Genentech, South San Francisco, Calif.) and also known as c2C4, is a monoclonal antibody that inhibits the dimerization of HER2 with other HER receptors.

Cetuximab, marketed as PERTUZUMAB® (ImClone, New York, N.Y.; and Bristol Myers Squibb, New York, N.Y.), is a chimeric (mouse/human) monoclonal antibody that binds to and inhibits EGF-R.

Gefitinib, marketed as IRESSA® (AstraZeneca, London; and Teva, Petah Tikva, Israel), is a drug that acts in a similar manner to erlotinib. Gefitinib selectively inhibits EGF-R's tyrosine kinase domain.

Neratinib (Pfizer Inc., New York, N.Y.) is an inhibitor of the HER2 receptor tyrosine kinase. Neratinib binds irreversibly to the HER2 receptor and thereby reduces autophosphorylation in cells, apparently by targeting a cysteine residue in the ATP-binding pocket of the receptor. Treatment of cells with neratinib results in inhibition of downstream signal transduction events and cell cycle regulatory pathways, arrest at the G1-S-phase transition of the cell cycle, and ultimately decreased cellular proliferation. In addition, neratinib inhibits the EGF-R kinase and proliferation of EGF-R-dependent cells.

In some embodiments, a method for treatment of HER3-associated disease can include administration of U1-1, U1-2, U1-3, U1-4, U1-5, U1-6, U1-7, U1-8, U1-9, U1-10, U1-11, U1-12, U1-13, U1-14, U1-15, U1-16, U1-17, U1-18, U1-19, U1-20, U1-21, U1-22, U1-23, U1-24, U1-25, U1-26, U1-27, U1-28, U1-29, U1-30, U1-31, U1-32, U1-33, U1-34, U1-35, U1-36, U1-37, U1-38, U1-39, U1-40, U1-41, U1-42, U1-43, U1-44, U1-45, U1-46, U1-47, U1-48, U1-49, U1-50, U1-51, U1-52, U1-53, U1-55.1, U1-55, U1-57.1, U1-57, U1-58, U1-59, U1-61.1, U1-61, or U1-62, U1-1, U1-2, U1-3, U1-4, U1-5, U1-6, U1-7, U1-8, U1-9, U1-10, U1-11, U1-12, U1-13, U1-14, U1-15, U1-16, U1-17, U1-18, U1-19, U1-20, U1-21, U1-22, U1-23, U1-24, U1-25, U1-26, U1-27, U1-28, U1-29, U1-30, U1-31, U1-32, U1-33, U1-34, U1-35, U1-36, U1-37, U1-38, U1-39, U1-40, U1-41, U1-42, U1-43, U1-44, U1-45, U1-46, U1-47, U1-48, U1-49, U1-50, U1-51, U1-52, U1-53, U1-55.1, U1-55, U1-57.1, U1-57, U1-58, U1-59, U1-61.1, U1-61, or U1-62, U1-49, U1-53 or U1-59, in combination with any of the above agents (e.g., simultaneously or separately), or U1-1, U1-2, U1-3, U1-4, U1-5, U1-6, U1-7, U1-8, U1-9, U1-10, U1-11, U1-12, U1-13, U1-14, U1-15, U1-16, U1-17, U1-18, U1-19, U1-20, U1-21, U1-22, U1-23, U1-24, U1-25, U1-26, U1-27, U1-28, U1-29, U1-30, U1-31, U1-32, U1-33, U1-34, U1-35, U1-36, U1-37, U1-38, U1-39, U1-40, U1-41, U1-42, U1-43, U1-44, U1-45, U1-46, U1-47, U1-48, U1-49, U1-50, U1-51, U1-52, U1-53, U1-55.1, U1-55, U1-57.1, U1-57, U1-58, U1-59, U1-61.1, U1-61, or U1-62, U1-1, U1-2, U1-3, U1-4, U1-5, U1-6, U1-7, U1-8, U1-9, U1-10, U1-11, U1-12, U1-13, U1-14, U1-15, U1-16, U1-17, U1-18, U1-19, U1-20, U1-21, U1-22, U1-23, U1-24, U1-25, U1-26, U1-27, U1-28, U1-29, U1-30, U1-31, U1-32, U1-33, U1-34, U1-35, U1-36, U1-37, U1-38, U1-39, U1-40, U1-41, U1-42, U1-43, U1-44, U1-45, U1-46, U1-47, U1-48, U1-49, U1-50, U1-51, U1-52, U1-53, U1-55.1, U1-55, U1-57.1, U1-57, U1-58, U1-59, U1-61.1, U1-61, or U1-62, U1-49, U1-53 or U1-59, in combination with any of the above agents and any other agent(s), for treatment of prostate conditions, including, e.g., BPH and prostate cancer. For sequence identification of the CDR's antibodies listed above, see, TABLE 1 and the sequence listing and disclosure of U.S. Pat. No. 7,705,130, and which is hereby incorporated herein by reference in its entirety.

In some embodiments, the second agent can be a chemotherapeutic drug. For example, agents that act as microtubule stimulants include NK-105 (paclitaxel) [(−)-(1S,2R,3S,4S,5R,7S,8S,10R,13S)-4,10-diacetoxy-2-benzoyloxy-5,20-epoxy-1,7-dihydroxy-9-oxotax-1′-en-13-yl (2R,3S)-3-benzoylamino-2-hydroxy-3-phenylpropionate] (NanoCarrier, Chiba, Japan), milataxel (1,10β-dihydroxy-9-oxo-5β,20-epoxy-3zeta-tax-11-ene-2α,4,7β13α-tetrayl 4-acetate 2-benzoate 13-[(2R,3R)-3-(tert-butoxycarbonylamino)-3-(furan-2-yl)-2-hydroxypropanoate]7-propanoate) (Taxolog, Fairfield, N.J.), laulimalide (Kosan Biosciences, Hayward, Calif. (B-M Squibb)), sarcodictyin A (3-(1-methylimidazol-4-yl)-2(E)-propenoic acid (1R,4aR,6S,7S,10R,12aR)-1′-methoxycarbonyl-7,10-epoxy-10-hydroxy-1-isopropyl-4,7-dimethyl-1,2,4a,5,6,7,10,12a-octahydrobenzocyclododecen-6-yl ester) (Pfizer, New York, N.Y.), simotaxel ((2aR,4S,4aS,6R,9S,11S,12S,12aR,12bS)-4,11-dihydroxy-4a,8,13,13-tetramethyl-5-oxo-2a,3,4,4a,5,6,9,10,11,12,12a,12b-dodecahydro-7,11-methano-1H-cyclodeca[3,4]benz[1,2-b]oxete-6,9,12,12b-tetrayl 12b-acetate 12-benzoate 6-cyclopentanecarboxylate 9-[(2R,3R)-2-hydroxy-3-[[(1-methylethoxy)carbonyl]amino]-3-(thiophen-2-yl)propanoate]) (Taxolog, Fairfield, N.J.), SYN-2001 (CLL Pharma, Nice, France), TL-310 (Taxolog, Fairfield, N.J.), TL1836 (Taxolog, Fairfield, N.J.), tesetaxel (2′-[(dimethyla-mino)methyl]-1-hydroxy-5β,20-epoxy-9α,10α-dihydro[1,3]dioxolo[4′,5′:9,10]tax-11-ene-2α,4,13α-triyl-4-acetate 2-benzoate 13-[(2R,3S)-3-[(tert-butoxycarbonyl)amino]-3-(3-fluoropyridin-2-yl)-2-hydroxypropanoate) (Daiichi Sankyo, Tokyo, Japan), TL-1892 (Taxolog, Fairfield, N.J.), TPI-287((2′R,3′S)-2′-hydroxy-N-carboxy-3′-amino-5′-methyl-hexanoic, N-tert-butyl ester, 13 ester 5β-20-epoxy-1,2α,4,7β,9α,10α,13α-heptahydroxy-4,10-diacetate-2-benzoate-7,9-acrolein acetal-tax-1′-ene (Tapestry Pharmaceuticals, Boulder, Colo.), ortataxel (2aR-[2aα,4β,4aβ,6β,9α(2R,3S),10α,11α,12α,12aα,12bα]-3-(tert-butoxycarbonyl-amino)-2-hydroxy-5-methyl-hexanoic acid 6,12b-diacetoxy-12-benzoyloxy-10,11-carbonyldioxy-4-hydroxy-4a,8,13,13-tetramethyl-5-oxo-2a,3,4,4a,5,6,9,10,11,12,12a,12b-dodecahydro-1H-7,11-methanocyclodeca[3,4]benz[1,2-b]oxet-9-yl ester) (Indena, Milan, Italy), paclitaxel poliglumex (L-pyroglutamylpoly-L-glutamyl-L-glutamic acid partially γ-esterified with (1R,2S)-2-(benzoylamino)-1-[[[(2aR,4S,4aS,6R,9S,11S,12S,12aR,12bS)-6,12b-bis(acetyloxy)-12-(benzoyloxy)-4,11-dihydroxy-4a,8,13,13-tetramethyl-5-oxo-2a,3,4,4a,5,6,9,10,11,12,12a,12b-dodecahydro-7,11-methano-1H-cyclodeca[3,4]benzo[1,2-b]oxet-9-yl]oxy]carbonyl]-2-phenylethyl) (Cell Therapeutics, Seattle, Wash.), paclitaxel protein-bound particles (paclitaxel: (−)-(1S,2R,3S,4S,5R,7S,8S,10R,13S)-4,10-diacetoxy-2-benzoyloxy-5,20-epoxy-1,7-dihydroxy-9-oxotax-11-en-13-yl (2R,3S)-3-benzoylamino-2-hydroxy-3-phenylpropionate) (Abraxis BioScience, Los Angeles, Calif.), paclitaxel(NCl)((−)-(1S,2R,3S,4S,5R,7S,8S,10R,13S)-4,10-diacetoxy-2-benzoyloxy-5,20-epoxy-1,7-dihydroxy-9-oxotax-1′-en-13-yl (2R,3S)-3-benzoylamino-2-hydroxy-3-phenylpropionate) (NCl(NIH)), paclitaxel (NeoPharm, Lake Bluff, Ill.) ((−)-(1S,2R,3S,4S,5R,7S,8S,10R,13S)-4,10-diacetoxy-2-benzoyloxy-5,20-epoxy-1,7-dihydroxy-9-oxotax-1′-en-13-yl (2R,3S)-3-benzoylamino-2-hydroxy-3-phenylpropionate) (NeoPharm, Lake Bluff, Ill.), patupilone((1S,3S,7S,10R,11S,12S,16R)-7,11-dihydroxy-8,8,10,12,16-pentamethyl-3-[(1E)-1-(2-methyl-1,3-thiazol-4-yl)prop-1-en-2-yl]-4,17-dioxabicyclo[14.1.0]heptadecane-5,9-dione) (US Publication No. 20030104625, Novartis, Basel, Switzerland), PEG-paclitaxel (Enzo Pharmaceuticals, Long Island, N.Y.), docetaxel hydrate((−)-(1S,2S,3R,4S,5R,7S,8S,10R,13S)-4-acetoxy-2-benzoyloxy-5,20-epoxy-1,7,10-trihydroxy-9-oxotax-1′-ene-13-yl(2R,3S)-3-tert-butoxycarbonylamino-2-hydroxy-3-phenylpropionate trihydrate) (Sanofi-Aventis, Bridgewater, N.J.), eleutherobin (3-(1-methylimidazol-4-yl)-2(E)-propenoic acid (1R,4aR,6S,7S,10R,12aR)-1′-(2-O-acetyl-β-D-arabinopyranosyloxymethyl)-7,10-epoxy-1-isopropyl-10-methoxy-4,7-dimethyl-1,2,4a,5,6,7,10,12a-octahydrobenzocyclododecen-6-yl ester) (Bristol-Myers Squibb, New York, N.Y.), IDN-5390 (Indena, Milan, Italy), ixabepilone ((1S,3S,7S,10R,11S,12S,16R)-7,11-dihydroxy-8,8,10,12,16-pentamethyl-3-[(1E)-1-methyl-2-(2-methylthiazol-4-yl)ethenyl]-17-oxa-4-azabicyclo[14.1.0]heptadecane-5,9-dione) (Bristol-Myers Squibb, New York, N.Y.), KOS-1584 (Kosan Biosciences, Hayward, Calif. (B-M Squibb)), KOS-1803 (17-iso-oxazole 26-trifluoro-9,10-dehydro-12,13-desoxy-epothilone B) (Kosan Biosciences, Hayward, Calif. (B-M Squibb)), KOS-862 (Kosan Biosciences, Hayward, Calif. (B-M Squibb); U.S. Pat. Nos. 6,204,388 and 6,303,342), larotaxel (1-hydroxy-9-oxo-513,20-epoxy-7β,19-cyclotax-11-ene-2α,4,10β,13α-tetrayl 4,10-diacetate 2-benzoate 13-[(2R,3S)-3-[(tert-butoxycarbonyl)amino]-2-hydroxy-3-phenylpropanoate]dehydrate) (Sanofi-Aventis, Bridgewater, N.J., PCT Publication Nos. WO 95/26961 and WO 96/1259), ANG-1005 (Angiopep-2/paclitaxel conjugate) (AngioChem, Montreal, Canada, U.S. Pat. No. 7,557,182), BMS-184476 (See: Bristol-Myers Squibb, New York, N.Y., EP Publication No. 639577), BMS-188797 (Bristol-Myers Squibb, New York, N.Y.), BMS-275183 (3′-tert-butyl-3′-N-tert-butyloxycarbonyl-4-deacetyl-3′-dephenyl-3′-N-debenzoyl-4-O-methyoxycarbonyl-paclitaxel) (Bristol-Myers Squibb, New York, N.Y.), BMS-310705 (Bristol-Myers Squibb, New York, N.Y.), BMS-753493 (Bristol-Myers Squibb, New York, N.Y.), cabazitaxel (1-hydroxy-7β,10β-dimethoxy-9-oxo-513,20-epoxytax-11-ene-2α,4,13α-triyl-4-acetate 2-benzoate 13-[(2R,3S)-3-[[(tertbutoxy)carbonyl]amino]-2-hydroxy-3-phenyl-propanoate]) (Sanofi-Aventis, Bridgewater, N.J.), DHA-paclitaxel (Protarga, King of Prussia, Pa., TAXOPREXIN®), disermolide ([3S-[3α,4β,5β,6α(2R*,3Z,5R*,6R*,7S*,8Z,11R*,12S*, 13S*,14S*,15R*,16E)]]-6-[14[(amino-carbonyl)oxy]-2,6,12-trihydroxy-5,7,9,11,13,15-hexamethyl-3,8,16,18-nonadecate-traenyl]tetrahydro-4-hydroxy-3,5-dimethyl-2H-pyran-2-one) (See: Novartis, Basel, Switzerland, U.S. Pat. Nos. 4,939,168 and 5,681,847). Some of these microtubule stimulants have a taxane ring in their chemical structures; such compounds having a taxane ring are referred as “taxanes” herein.

Anthracyclins include actinomycins such as actinomycin D (Dactinomycin: 2-amino-N,N′-bis[(6S,9R,10S,13R,18a5)-6,13-diisopropyl-2,5,9-trimethyl-1,4,7,11,14-penta-oxohexadecahydro-1H-pyrrolo[2,1-i][1,4,7,10,13]oxatetraazacyclohexadecin-10-yl]-4,6-dimethyl-3-oxo-3H-phenoxazine-1,9-dicarboxamide), bleomycin (bleomycin hydrochloride: (3-{[(2′-{(5S,8S,9S,10R,13S)-15-{6-amino-2-[(1S)-3-amino-1-{[(2S)-2,3-diamino-3-oxopropyl]amino}-3-oxopropyl]-5-methylpyrimidin-4-yl}-13-[{[(2R,3S,4S,5S,6S)-3-{[(2R,3S,4S,5R,6R)-4-(carbamoyloxy)-3,5-dihydroxy-6-(hydroxymethyl)tetrahydro-2H-pyran-2-yl]oxy}-4,5-dihydroxy-6-(hydroxymethyl)tetrahydro-2H-pyran-2-yl]oxy}(1H-imidazol-5-yl)methyl]-9-hydroxy-5-[(1R)-1-hydroxyethyl]-8,10-dimethyl-4,7,12,15-tetraoxo-3,6,11,14-tetraaza-pentadec-1-yl}-2,4′-bi-1,3-thiazol-4-yl)carbonyl]amino}propyl)(dimethyl)sulfonium), daunorubicin hydrochloride (daunorubicin: 85-cis)-8-Acetyl-10-((3-amino-2,3,6-trideoxy-alpha-L-lyxo-hexopyranosyl)oxy)-7,8,9,10-tetrahydro-6,8,11-trihydroxy-1-methoxy-5,12-naphthacenedione hydrochloride), doxorubicin hydrochloride (doxorubicin: (8S,10S)-10-[(3-amino-2,3,6-trideoxy-α-L-lyxo-hexopyranosyl)oxy]-8-glycoloyl-7,8,9,10-tetrahydro-6,8,11-trihydroxy-1-methoxy-5,12-naphthacenedione hydrochloride) (Alza, Mountain View, Calif.), idarubicin hydrochloride ((7S,9S)-9-acetyl-7,8,9,10-tetrahydro-6,7,9,11-tetrahydroxy-7-O-(2,3,6-trideoxy-3-amino-α-L-lyxo-hexopyranosyl)-5,12-naphthacenedione hydrochloride) (See: Pfizer, New York, N.Y., U.S. Pat. Nos. 4,046,878 and 4,471,052), and mitomycin ((1aS,8S,8aR,8bR)-6-Amino-4,7-dioxo-1,1a,2,8,8a,8b-hexahydro-8a-methoxy-5-methylazirino[2,3:3,4]pyrrolo[1,2-α]indol-8-ylmethylcarbamate) (Kyowa-Hakko-Kirin, Tokyo, Japan).

Cisplatin and gemcitabine are additional examples of chemotherapeutic agents. Cisplatin or cis-diamminedichloroplatinum(II) is a platinum-based drug used to treat various types of cancers. The cisplatin platinum complex reacts in vivo, binding to and causing crosslinking of DNA, which ultimately triggers apoptosis. Gemcitabine is a nucleoside analog in which the hydrogen atoms on the 2′ carbons of deoxycytidine are replaced by fluorine atoms. Like fluorouracil and other pyrimidine analogues, gemcitabine replaces cytidine during DNA replication, which arrests tumor growth since further nucleosides cannot be attached to the “faulty” nucleoside, resulting in apoptosis. Gemcitabine is marketed as GEMZAR® by Eli Lilly and Company (Indianapolis, Ind.). In some embodiments, a combination for treatment of HER3-associated disease can be: U1-49, U1-53 or U1-59 in combination with a second agent as described herein and cisplatin or gemcitabine and other agent(s), for treatment of cancer which is gastrointestinal cancer, pancreatic cancer, prostate cancer, ovarian cancer, stomach cancer, endometrial cancer, salivary gland cancer, kidney cancer, colon cancer, thyroid cancer, bladder cancer, glioma, melanoma, lung cancer including non-small cell lung cancer, colorectal cancer and/or breast cancer including metastatic breast cancer.

Capecitabine (pentyl[1-(3,4-dihydroxy-5-methyl-tetrahydrofuran-2-yl)-5-fluoro-2-oxo-1H-pyrimidin-4-yl]aminomethanoate, Xeloda, Roche) is an orally-administered chemotherapeutic agent. Capecitabine is a prodrug that is enzymatically converted to 5-fluorouracil in the tumor, where it inhibits DNA synthesis and slows growth of tumor tissue. In some embodiments, a combination for treatment of HER3-associated disease can be: U1-49, U1-53 or U1-59 in combination with a second agent as described herein (e.g., lapatanib) and capecitabine for treatment of cancer, wherein the cancer is gastrointestinal cancer, pancreatic cancer, prostate cancer, ovarian cancer, stomach cancer, endometrial cancer, salivary gland cancer, kidney cancer, colon cancer, thyroid cancer, bladder cancer, glioma, melanoma, lung cancer including non-small cell lung cancer, colorectal cancer and/or breast cancer including metastatic breast cancer. In some cases, such a combination can be administered after failure of prior treatment with an anthracyclin or taxane, for example.

Docetaxel((2R,3S)—N-carboxy-3-phenylisoserine, N-tert-butyl ester, 13-ester with 5,20-epoxy-1,2,4,7,10,13-hexahydroxytax-1′-en-9-one 4-acetate 2-benzoate, trihydrate) and paclitaxel((2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-1′-en-2-yl be) are chemotherapeutic agents. Docetaxel is marketed as Taxotere by Sanofi Aventis. Paclitaxel is marketed as Taxol by Bristol-Myers Squibb. In the formulation of Taxol, paclitaxel is dissolved in Cremophor EL and ethanol, as a delivery agent. A formulation in which paclitaxel is bound to albumin is marketed as Abraxane. In some embodiments, a combination for treatment of HER3-associated disease can be: U1-49, U1-53 or U1-59 in combination with a second agent as described herein (e.g., trastuzumab) and docetaxel or paclitaxel and other agent(s) such as trastuzumab, for treatment of cancer, wherein the cancer is gastrointestinal cancer, pancreatic cancer, prostate cancer, ovarian cancer, stomach cancer, endometrial cancer, salivary gland cancer, kidney cancer, colon cancer, thyroid cancer, bladder cancer, glioma, melanoma, lung cancer including non-small cell lung cancer, colorectal cancer and/or breast cancer including metastatic breast cancer.

Doxorubicin hydrochloride liposome injection is marketed as Doxil, a liposome formulation comprising doxorubicin chloride. In some embodiments, a combination treatment for HER3-associated disease can include administering U1-49, U1-53 or U1-59 in combination with a second agent as described herein and doxorubicin hydrochloride liposome injection, with or without one or more other agents such as paclitaxel or platinum-based chemotherapeutic agents, for treatment of cancer such as breast cancer, gastro-intestinal cancer, pancreatic cancer, prostate cancer, ovarian cancer, stomach cancer, endometrial cancer, salivary gland cancer, lung cancer, renal cancer, colon cancer, colorectal cancer, thyroid cancer, bladder cancer, glioma, melanoma, metastatic breast cancer, non-small cell lung cancer, epidermoid carcinoma, fibrosarcoma, melanoma, nasopharyngeal carcinoma, and squamous cell carcinoma.

Irinotecan hydrochloride hydrate (irinotecan: (+)-(4S)-4,1′-diethyl-4-hydroxy-9-[(4-piperidinopiperidino)carbonyloxy]-1H-pyrano[3′,4′: 6,7]indolizino[1-2-b]quinoline-3,14(4H,12H)-dione hydrochloride trihydrate) (See: Yakult, EP Publication Nos. 137145 and 56692) is marketed as Campto, Camptosar and Ircan. In some embodiments, a combination treatment for HER3-associated disease can include administering U1-49, U1-53 or U1-59 in combination with a second agent as described herein and irinotecan hydrochloride hydrate, or U1-49, U1-53 or U1-59 in combination with a second agent as described herein, irinotecan hydrochloride hydrate, and one or more other agent(s) such as 5-FU(5′-deoxy-5-fluorouridine or 5-fluoro-2,4(1H,3H)-pyrimidinedione), calcium folinate (N-[4-[[(2-amino-5-formyl-1,4,5,6,7,8-hexahydro-4-oxo-6-pteridinyl)methylamino]benzoyl]-L-glutamic acid calcium salt (1:1)) or calcium levofolinate ((−)-calcium N-[4-[[[(6S)-2-amino-5-formyl-1,4,5,6,7,8-hexahydro-4-oxo-6-pteridinyl]methyl]amino]benzoyl]-L-glutamate), and combinations thereof, for treatment of cancer such as breast cancer, gastrointestinal cancer, pancreatic cancer, prostate cancer, ovarian cancer, stomach cancer, endometrial cancer, salivary gland cancer, lung cancer, renal cancer, colon cancer, colorectal cancer, thyroid cancer, bladder cancer, glioma, melanoma, metastatic breast cancer, non-small cell lung cancer, epidermoid carcinoma, fibrosarcoma, melanoma, nasopharyngeal carcinoma, and squamous cell carcinoma.

As further described below, these and other agents can be contained within the compositions provided herein, and can be administered in a variety of different forms, combinations and dosages.

### 5. Compositions

HER3 binding agents as described herein can be incorporated into compositions for treatment of a prostate condition such as BPH or prostate cancer. Thus, this document also provides the use of a HER3 binding agent in the manufacture of a medicament for treating BPH or prostate cancer, for example. The compositions can further include, for example, one or more pharmaceutically acceptable carriers, diluents and/or adjuvants, as well as a second agent (e.g., an agent that binds to another HER family member, or a chemotherapeutic agent). The term “pharmaceutical composition,” as used herein, refers to a chemical compound or composition capable of inducing a desired therapeutic effect when properly administered to a patient (See: The McGraw-Hill Dictionary of Chemical Terms, Parker, Ed., McGraw-Hill, San Francisco (1985)). The potency of the pharmaceutical compositions provided herein typically is based on the binding of the at least one binding protein to HER3. In some embodiments, this binding can lead to a reduction of the HER3-mediated signal transduction.

A “pharmaceutically acceptable carrier” (also referred to herein as an “excipient” or a “carrier”) is a pharmaceutically acceptable solvent, suspending agent, stabilizing agent, or any other pharmacologically inert vehicle for delivering one or more therapeutic compounds (e.g., HER binding proteins) to a subject, which is nontoxic to the cell or mammal being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10 residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylgly-cerol, or dioleoyl phosphatidylethanolamine. Numerous lipophilic agents are commercially available, including LIPOFECTIN® (Invitrogen/Life Technologies, Carlsbad, Calif.) and EFFECTENE™ (Qiagen, Valencia, Calif.).

In some embodiments, at least one of the agents contained in a pharmaceutical composition (e.g., a HER3 binding agent or an agent that binds and/or inhibits another HER family member) can be coupled to an effector such as calicheamicin, duocarmycins, auristatins, maytansinoids, a radioisotope, or a toxic chemotherapeutic agent such as geldanamycin and maytansine. Such conjugates can be particularly useful for targeting cells (e.g., cancer cells) expressing HER3.

Linking binding proteins to radioisotopes can provide advantages to tumor treatments. Unlike chemotherapy and other forms of cancer treatment, radioimmunotherapy or the administration of a radioisotope-binding protein combination can directly target cancer cells with minimal damage to surrounding normal, healthy tissue. With this “magic bullet,” patients can be treated with much smaller quantities of radioisotopes than other forms of treatment available today. Suitable radioisotopes include, for example, yttrium⁹⁰ (⁹⁰Y), indium¹¹¹ (¹¹¹In), ¹³¹I, ⁹⁹mTc, radiosilver-111, radiosilver-199, and Bismuth²¹³. The linkage of radioisotopes to binding proteins may be performed with, for example, conventional bifunctional chelates. Since silver is monovalent, for radiosilver-111 and radiosilver-199 linkage, sulphur-based linkers may be used (See: Hazra et al., Cell Biophys. 24-25:1-7 (1994)). Linkage of silver radioisotopes may involve reducing the immunoglobulin with ascorbic acid. Furthermore, tiuxetan is an MX-DTPA linker chelator attached to ibritumomab to form ibritumomab tiuxetan (Zevalin) (See: Witzig, Cancer Chemother. Pharmacol. 48 (Suppl 1):91-95 (2001)). Ibritumomab tiuxetan can react with radioisotypes such as indium¹¹¹ (¹¹¹In) or ⁹⁰Y to form ¹¹¹In-ibritumomab tiuxetan and ⁹⁰Y-ibritumomab tiuxetan, respectively.

The binding proteins described herein, particularly when used to treat cancer, can be conjugated with toxic chemotherapeutic drugs such as maytansinoids, (See: Hamann et al., Bioconjug. Chem. 13:40-46, (2002)), geldanamycinoids (See: Mandler et al., J. Natl. Cancer Inst. 92:1549-1551 (2000)) and maytansinoids, for example, the maytansinoid drug, DM1 (See: Liu et al., Proc. Natl. Acad. Sci. USA, 93:8618-8623, (1996)). Linkers that release the drugs under acidic or reducing conditions or upon exposure to specific proteases may be employed with this technology. A binding protein may be conjugated as described in the art.

In some embodiments, a binding protein can be conjugated to auristatin-PE. Auristatin-PE, e.g., is an antimicrotubule agent that is a structural modification of the marine, shell-less mollusk peptide constituent dolastatin 10. Auristatin-PE has both anti-tumor activity and anti-tumor vascular activity (See: Otani et al., Jpn. J. Cancer Res. 91:837-44 (2000)). For example, auristatin-PE inhibits cell growth and induces cell cycle arrest and apoptosis in pancreatic cancer cell lines (See: Li et al., Int. J. Mol. Med. 3:647-53 (1999)). Accordingly, to specifically target the anti-tumor activity and anti-tumor vascular activities of auristatin-PE to particular tumors, auristatin-PE may be conjugated to a binding protein as provided herein.

The pharmaceutical compositions provided herein also can contain at least one further active agent. Examples of further active agents include antibodies or low molecular weight inhibitors of other receptor protein kinases, such as IGFR-1 and c-met, receptor ligands such as vascular endothelial factor (VEGF), cytotoxic agents such as doxorubicin, cisplatin or carboplatin, cytokines, or anti-neoplastic agents. Many anti-neoplastic agents are known in the art. In some embodiments, an anti-neoplastic agent can be selected from the group of therapeutic proteins including, but not limited to, antibodies and immunomodulatory proteins. In some embodiments, an anti-neoplastic agent can be selected from the group of small molecule inhibitors and chemotherapeutic agents consisting of mitotic inhibitors, kinase inhibitors, alkylating agents, anti-metabolites, intercalating antibiotics, growth factor inhibitors, cell cycle inhibitors, enzymes, topoisomerase inhibitors, histone deacetylase inhibitors, anti-survival agents, biological response modifiers, anti-hormones (e.g., anti-androgens), microtubule stimulants, anthracyclins, and anti-angiogenesis agents. When the anti-neoplastic agent is radiation, treatment can be achieved either with an internal source (e.g., brachytherapy) or an external source (e.g., external beam radiation therapy). The one or more further active agent(s) can be administered with the HER3-binding agent and the second agent either simultaneously or separately, in a single formulation or in individual (separate) formulations for each active agent.

The pharmaceutical compositions provided herein can be especially useful for diagnosis, prevention, or treatment of a prostate condition (e.g., a prostate condition associated with increased HER family signal transduction). The condition can be, for example, associated with increased HER3 phosphorylation, increased complex formation between HER3 and other members of the HER family, increased PI₃ kinase activity, increased c-jun terminal kinase activity and/or AKT activity, increased ERK2 and/or PYK2 activity, or any combination thereof. The prostate condition can be, e.g., BPH or prostate cancer.

Pharmaceutical compositions can be formulated by mixing one or more active agents with one or more physiologically acceptable carriers, diluents, and/or adjuvants, and optionally other agents that are usually incorporated into formulations to provide improved transfer, delivery, tolerance, and the like. A pharmaceutical composition can be formulated, e.g., in lyophilized formulations, aqueous solutions, dispersions, or solid preparations, such as tablets, dragees or capsules. A multitude of appropriate formulations can be found in the formulary known to all pharmaceutical chemists: Remington's Pharmaceutical Sciences (18^{th} ed, Mack Publishing Company, Easton, Pa. (1990), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. Also See: Baldrick, Regul. Toxicol. Pharmacol. 32:210-218 (2000); Wang, Int. J. Pharm. 203:1-60 (2000); Charman, J. Pharm. Sci. 89:967-978 (2000); and Powell et al., PDA J. Pharm. Sci. Technol. 52:238-311 (1998), and the citations therein for additional information related to formulations, excipients and carriers well known to pharmaceutical chemists.

Methods for formulating and subsequently administering therapeutic compositions are well known to those skilled in the art. Dosing generally is dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual polypeptides, and can generally be estimated based on EC₅₀ found to be effective in in vitro and in vivo animal models. Typically, dosage is from about 0.01 μg to about 100 μg per kg of body weight, and may be given once or more daily, biweekly, weekly, monthly, or even less often. Following successful treatment, it may be desirable to have the patient undergo maintenance therapy to prevent the recurrence of the disease state.

In various embodiments dosage may be from about 0.01 to about 0.1 μg per kg of body weight, from about 0.1 to about 1 μg per kg of body weight, from about 1 to about 10 μg per kg of body weight, from about 10 to about 100 μg per kg of body weight or greater than about 100 μg per kg of body weight.

Pharmaceutical compositions can be administered by a number of methods, depending upon whether local or systemic treatment is desired and upon the area to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, HER3 binding proteins can be administered by injection or infusion into the cerebrospinal fluid, typically with one or more agents capable of promoting penetration of the polypeptides across the blood-brain barrier.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

HER binding agents can further encompass any pharmaceutically acceptable salts, esters, or salts of such esters, or any other compound which, upon administration to an animal including a human, is capable of providing (directly or indirectly) the biologically active metabolite or residue thereof. Accordingly, for example, this document provides pharmaceutically acceptable salts of small molecules and polypeptides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the polypeptides provided herein (i.e., salts that retain the desired biological activity of the parent polypeptide without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); and salts formed with organic acids (e.g., acetic acid, citric acid, oxalic acid, palmitic acid, or fumaric acid).

Some embodiments provided herein include pharmaceutical compositions containing one or more HER3 binding agents, with or without one or more second agents (e.g., one or more that bind to another HER family member, or one or more chemotherapeutic agents), and one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions. Other non-polypeptide agents (e.g., chemotherapeutic agents) also are within the scope of this document. Such combined compounds can be used together or sequentially.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

The pharmaceutical formulations, which can be presented conveniently in unit dosage form, can be prepared according to conventional techniques well known in the pharmaceutical industry. Such techniques include the step of bringing into association the active ingredients (e.g., the HER family binding agents provided herein) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the polypeptide contained in the formulation.

The compositions described herein can be formulated into any of many possible dosage forms such as, but not limited to, tablets, capsules, liquid syrups, soft gels, suppositories, and enemas. The compositions also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

HER binding agents can be combined with packaging material and sold as kits for treating prostate conditions. Components and methods for producing articles of manufacture are well known. The articles of manufacture may combine one or more of the polypeptides and compounds set out in the above sections. In addition, the article of manufacture further may include, for example, buffers or other control reagents for reducing or monitoring reduced immune complex formation. Instructions describing how the polypeptides are effective for treating prostate conditions can be included in such kits.

### 6. Methods

This document also provides methods for treating or preventing a prostate condition (e.g., BPH or prostate cancer) in a subject. For example, a method can include administering to a subject (e.g., a mammal such as a human) a HER3 binding agent or a composition containing a HER3 binding agent, as described herein. The methods can be used to, for example, treat or prevent BPH in a subject in need thereof, reduce prostate weight in a subject, treat or prevent prostate cancer; inhibit or reduce growth of prostate cancer cells, or prevent, reduce, or reverse androgen independent growth and/or proliferation of prostate cancer cells.

The term “treatment or prevention,” when used herein, refers to both therapeutic treatment and prophylactic or preventative measures, which can be used to prevent, slow, or lessen the effects of the targeted condition or disorder. Those in need of prevention or treatment can include those already having the disorder, as well as those who may be likely to develop the disorder, or those in whom the disorder is to be prevented. The subject in need of prevention or treatment can be a mammalian patient (i.e., any animal classified as a mammal, including humans, domestic and farm animals, and zoo, sports, or pet animals, such as dogs, cats, cattle, horses, sheep, pigs, goats, and rabbits). In some embodiments, the subject in need of treatment is a human patient.

Methods for preventing or treating prostate conditions in subject in need thereof can include administering to the subject an effective amount of at least one HER3 binding agent as described herein. In some cases, the methods also can include administering at least one other agent (e.g., an agent against another HER family member, or a chemotherapeutic compound, as described above). Such treatment can, for example, inhibit abnormal cell growth, migration or invasion. When a combination of agents is administered, they can be administered simultaneously (e.g., when they are contained in the same composition, or by admixture into a common i.v. bag), or separately (e.g., sequentially).

As used herein, the term “effective amount” is an amount of an agent that results in a decrease or stabilization in one or more symptoms or clinical characteristics of the prostate condition being treated. For example, administration of an effective amount of a composition as described herein can result in decreased prostate size (e.g., as determined by weight or diameter), slowing of tumor growth, decreased tumor size, or decreased activation of HER3 or HER3-responsive biomarkers (e.g., Akt, HER2, ERK, or EGF-R). The decrease or slowing can be any reduction as compared to a previous value (e.g., a 5%, 10%, 20%, 25%, or more than 25% reduction in symptom or characteristic). In some embodiments, an “effective amount” can result in stable disease.

In addition to classical modes of administration of potential binding protein therapeutics, e.g., via the above mentioned formulations, newly developed modalities of administration may also be useful. For example, local administration of ¹³¹I-labeled monoclonal antibody for treatment of primary brain tumors after surgical resection has been reported. Additionally, direct stereotactic intracerebral injection of monoclonal antibodies and their fragments is also being studied clinically and pre-clinically. Intracarotid hyperosmolar perfusion is an experimental strategy to target primary brain malignancy with drug conjugated human monoclonal antibodies.

As described above, the dose of the agents administered can depend on a variety of factors. These include, for example, the nature of the agents, the tumor type, and the route of administration. It should be emphasized that the present methods are not limited to any particular doses. Methods for determining suitable doses are known in the art.

Depending on the type and severity of the condition to be treated, up to about 20 mg/kg of HER binding agent can be administered to a patient in need thereof, e.g., by one or more separate administrations or by continuous infusion. A typical daily dosage might range from about 1 μg/day to about 100 mg/day or more, depending on the factors mentioned above. For repeated administrations over several days or longer, depending on the condition to be treated, the treatment can be sustained until a desired suppression of disease symptoms occurs.

In some cases, a method as provided herein can include one or more steps for monitoring the therapeutic outcome of the treatment. For example, a subject can be monitored for symptoms of their disease, to determine whether a reduction in symptoms has occurred. The subject also can be monitored, for example, for potential side effects of the treatment. The monitoring can be done after the administration step, and, in some embodiments, can be done multiple times (e.g., between administrations, if dosages are given more than once). Such methods can be used to assess efficacy and safety of the treatment methods described herein, for example.

The invention will be further described in the following examples, which do not limit the scope of the invention described in the claims.

### EXAMPLES

### Example 1

### HER3 Expression is Increased During Androgen Withdrawal

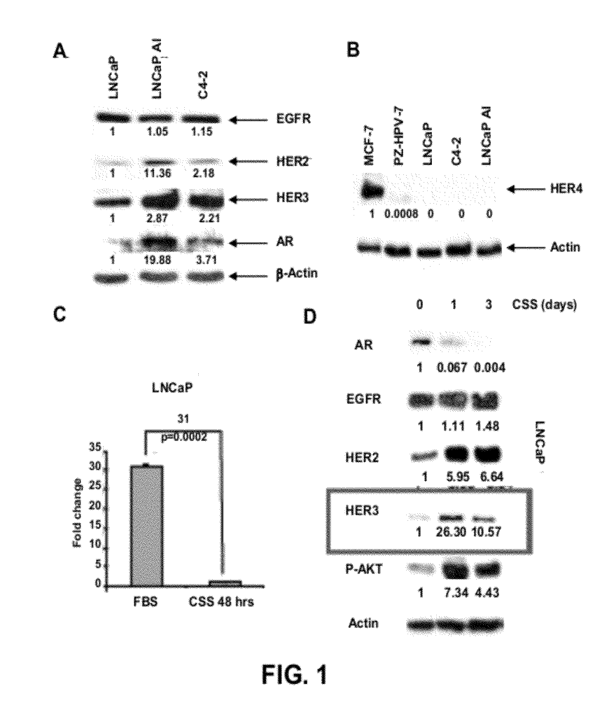

U1-59 is a fully human anti-HER3 monoclonal antibody that has in vivo anti-tumor activities in various types of human cancers (See: Treder et al., “Fully human Anti-HER3 monoclonal antibodies (mAbs) inhibit oncogenic signaling and tumor cell growth in vitro and in vivo,” AACR Annual Meeting, San Diego, Calif. (2008); and Freeman et al., “Fully human Anti-HER3 monoclonal antibodies (mAbs) have unique in vitro and in vivo functional and antitumor activities versus other HER family inhibitors,” AACR Annual Meeting, San Diego, Calif. (2008)). This antibody may be useful to inhibit proliferation and xenograft tumor growth, as well as to restore androgen dependence, in various prostate cancer cell lines. Androgen dependent LNCaP cells become androgen independent (AI) when cultured over extended periods of time in medium containing low androgen levels (See: Murillo et al., (supra) (2008); and Hsieh et al., Cancer Res. 53(12):2852-2857 (1993)). Comparison of LNCaP cells with their AI sublines LNCaP-AI and C4-2 showed that the AI sublines overexpressed HER2 and HER3 but not EGFR (FIG. 1A), while HER4 levels were extremely low (FIG. 1B). The increase in HER2 and HER3 was associated with androgen withdrawal. LNCaP cells were plated in medium containing FBS and then switched to low-androgen medium containing charcoal stripped serum. As shown in FIG. 1C, this treatment caused a sharp decrease in AR transcriptional activity from a PSA promoter construct, and also in AR expression (FIG. 1D, upper panel). There was a significant increase in the expression of HER2 (3^{rd} panel), HER3 (4^{th} panel), but not EGF-R. In addition, despite the lack of functional PTEN (a negative regulator of PI3K activation), LNCaP cells still experienced an increase in Akt phosphorylation (5^{th} panel).

### Example 2

### HER3 Overexpression Induces LNCaP Propagation in the Absence of Androgens

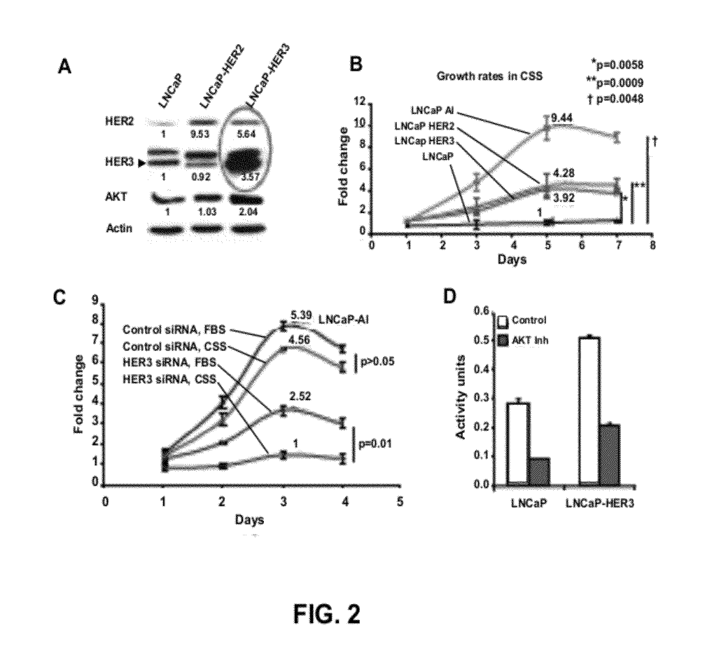

LNCaP cells were stably transfected with cDNA encoding HER2 or HER3 (pcDNA3-HER2 and pcDNA3-HER3) to generate the stable cell lines LNCaP-HER2 and LNCaP-HER3 (FIG. 2A). Multiple clones were screened to select those that expressed HER2 and HER3 at levels comparable to the levels in LNCaP-AI cells. Significantly, overexpression of HER3 also induced the overexpression of HER2. Overexpression of both HER2 and HER3 resulted in an increase in proliferation in low-androgen media versus the untransfected control cell line (FIG. 2B). On the other hand, transfection of HER3 siRNA inhibited proliferation in LNCaP-AI cells in both FBS and charcoal stripped serum (FIG. 2C). Inhibition of Akt prevented proliferation caused by HER3 overexpression (FIG. 2D). Thus, HER3 regulates cell growth in an Akt-dependent manner.

### Example 3

### HER3 Regulates Proliferation and Androgen Dependence in Prostate Cancer

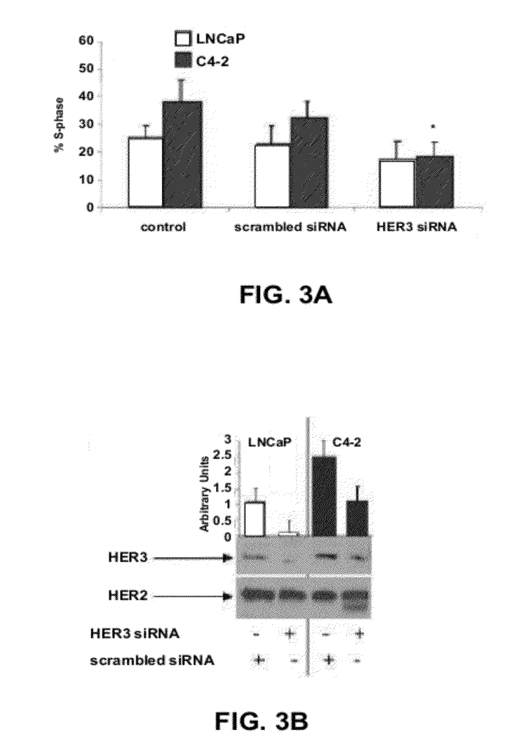

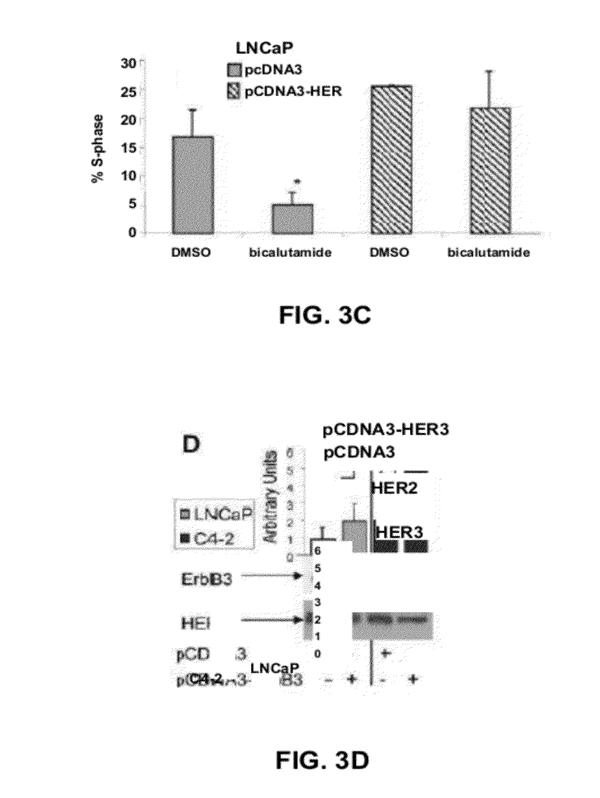

Flow cytometric analyses to evaluate the percentage of cells in S-phase revealed that transfection with HER3-specific siRNA, but not a scrambled siRNA, reduced proliferation rates in both LNCaP and C4-2 cells (FIG. 3A), indicating that HER3 is necessary for proliferation of these cells. As before, C4-2 cells expressed higher levels of HER3 compared to LNCaP cells (approximately 2×), and HER3-specific siRNA resulted in the downregulation of HER3 expression in both cell lines (FIG. 3B). Cells were transfected with 10 M siRNA because it appeared that this amount of HER3-specific siRNA duplex downregulated HER3 levels in C4-2 cells back to the levels seen in LNCaP cells. Co-transfection with -galactosidase followed by -gal assay showed similar levels of transfection efficiency in all cases. Similarly, overexpression of HER3 using a pcDNA3-HER3 plasmid increased proliferation rates in LNCaP cells (FIG. 3C) as well as C4-2 cells (data not shown). Importantly, flow cytometric analysis revealed that LNCaP cells transfected with an empty vector were growth-inhibited by treatment for 48 hours with the AR antagonist bicalutamide (5 M), indicating androgen dependence. In contrast, bicalutamide did not significantly reduce proliferation in cells that overexpress HER3, indicating androgen-independence (FIG. 3C). HER3 overexpression was confirmed by western blotting, and showed that LNCaP cells transfected with 2 g pcDNA3-HER3 increased HER3 levels to that of C4-2 cells (FIG. 3D). As before, transfection efficiency was determined by co-transfection of -gal, and -gal assays confirmed similar transfection efficiencies in all cases.

### Example 4

### Does Inhibition of HER3 Inhibit Proliferation Rates and Confer Androgen Dependence to Androgen-Independent Prostate Cancer Cells?

HER3 is overexpressed in prostate cancer relative to normal prostate (See: Chaib et al., supra (2001)). The Examples above show that HER3 is overexpressed in androgen independent clones of LNCaP cells compared to the androgen dependent parental cell line. Further, overexpression of HER3 in LNCaP cells conferred androgen independence, while inhibition of HER3 reduced proliferation. Culture in the absence of androgens (in medium containing charcoal stripped serum) and treatment with the anti-androgen bicalutamide inhibits cell proliferation in LNCaP cells but not in C4-2 cells (See: Mikhailova et al., Adv. Exp. Med. Biol. 617:397-405 (2008); and Wang et al., Oncogene 26(41):6061-6070 (2007); and Wang et al., Oncogene 27(56):7106-7117 (2008)). Transfection of HER3 cDNA into LNCaP cells prevented this inhibition, and also induced LNCaP cells to proliferate in the absence of androgens, HER3-specific siRNA prevented androgen independent growth in androgen-independent sublines of LNCaP cells.

Further studies are conducted to determine whether inhibition of HER3 using the fully human anti-HER3 monoclonal antibody, U1-59, inhibits proliferation in androgen-independent cells and reverts the cells back to an androgen-dependent phenotype. LNCaP cells or the androgen independent sublines C4-2 and LNCaP-AI, as well as “normal-like cells” RWPE1 and pRNS-1-1 (Shi et al. (2007) Prostate 67(6):591-602) and androgen independent cells such as MDA-PCa-2b and CWR22Rv1, are treated with U1-59 or vehicle as per manufacturer's recommended protocol at a range of concentrations to determine the optimum concentration needed to inhibit HER3 oncogenic signaling. The rate of cell growth is determined by MTT (3-(4,5-Dimethylthiazol-2-yl)-2,5-diphenyltetrazolium bromide) assay, and the rate of proliferation vs. apoptosis is determined by flow cytometry followed by Western blotting to determine the effect of the drug on cell cycle proteins (e.g., phosphorylation of cyclins D1, E, A, cdk2, and cdk4/6, and expression of p27 and p21), as well as on proteins involved in apoptosis (e.g., Bcl2, BclXL, BAD, and BAX).

To determine the effect of U1-59 on AR transcriptional activity, levels of AR and PSA expression are determined by Western blotting in cells that express PSA. AR transcriptional activity also is evaluated by transfecting cells with hPSA-luc, a luciferase-tagged PSA promoter region expressing two AR binding regions (ARE), and treating transfected cells with vehicle or antibody and then carrying out luciferase assays to determine AR transcriptional activity. Assays also are conducted to evaluate effects of the antibody on cell motility and invasiveness, growth in soft agar, and effects on downstream effectors of HER3 (i.e., the Akt and ERK signaling pathways).

To determine whether U1-59 retards androgen independence, the growth of cells in FBS vs. charcoal stripped serum is assessed in the presence or absence of bicalutamide, together with the antibody or vehicle. HER3 inhibition is detected in the cells by Western blotting in plasma membrane fractions obtained by Triton X-114 fractionation as previously described (See: Ghosh et al., Oncogene 18(28):4120-4130 (1999); Ghosh et al., Biochim. Biophys. Acta 1359(1):13-24 (1997); and Ghosh et al., J. Cell. Biochem. 74(4):532-543 (1999)). Freshly synthesized active HER3 localizes to the membrane and lack of HER3 in the membrane fraction is indicative of effective HER3 silencing (See: Walters et al., Oncogene 22(23):3598-3607 (2003)). The other half of the cells is fixed in 70% ethanol, stained with PI and the % cells in S-phase detected by flow cytometry (See: Ghosh et al., Cancer Res. 62(9):2630-2636 (2002)). It is expected that inhibition of HER3 will make the cells responsive to bicalutamide.

### Example 5

### Does Inhibition of HER3 During Androgen Withdrawal Prevent Development of Androgen Independent Tumors in Animal Models of Prostate Cancer?

Increased HER3 expression is associated with androgen independence in vitro, and HER3 levels are increased during androgen withdrawal. In androgen dependent cells, HER3 levels are negatively regulated by the androgen receptor. On the other hand, increased HER3 levels prevent androgen regulation of HER3. Studies are conducted to determine whether inhibition of HER3 in vivo during androgen withdrawal will prevent androgen independent growth of prostate cancer cells. An appropriate model of prostate cancer recurrence, the CWR22 model, is available for studies in vivo. CWR22 tumors are easily established in nude mice, are androgen dependent in the primary tumor state, and undergo rapid regression upon castration of the host (See: Nagabhushan et al., Cancer Res. 56(13):3042-3046 (1996)). Within 3-4 months, however, androgen independent tumors arise in the host animals, thus replicating the effect seen in human patients. The CWR22 recurrence model therefore is used to determine the effect of U1-59 on tumor development and tumor regression.

To determine whether inhibition of HER3 prevents tumor incidence, 4- to 5-week old nude/nude athymic male mice (Harlan, Indianapolis) are implanted with testosterone pellets prior to subcutaneous injections in both flanks of 20 million cells each extracted from CWR22 tumors in a 1:1 mixture with 50% Matrigel (n=40). Three weeks after implantation (or when palpable tumors are observed), the mice are castrated. These animals are followed for up to 12 months following surgery. It is expected that tumors will regress initially but will recur within 8-9 months in 50% of the animals. Growth of tumors in castrated mice indicates hormone-refractory growth. Mice bearing recurrent tumors are divided into two groups (n=20 tumors/group) that receive vehicle or U1-59, respectively. Treatment is initiated when the size of the recurrent tumor reaches 150 mm³. Tumor size is measured every other day with digital calipers, and tumor volume is calculated. Growth inhibition is calculated by tumor volume of treated mice divided by tumor volume of control mice (T/C). The effect of antibody treatment on tumor volume is followed for up to 12 weeks following the start of treatment. At the end of the study, all remaining tumors are collected, and recurrent tumors are weighed and bisected. One part was fixed in 10% formalin for immunohistochemistry, and the rest are quick frozen (isopropanol in liquid nitrogen).

Experiments also are conducted to determine whether inhibition of HER3 at the time of androgen withdrawal prevents recurrence, by determining whether progression of prostate cancer cells to androgen independence in castrated nude mice can be prevented by prior treatment with U1-59. Mice (n=40) are subcutaneously and bilaterally injected with CWR22 cells as described above, and the animals are castrated when palpable tumors are observed (approximately three weeks after injection). In these experiments, however, the castrated animals are treated with (i) vehicle or (ii) U1-59 (n=20/group) in the days following castration, well before the onset of recurrent tumors. This protocol is maintained for up to 9 months, or whenever the control animals begin to develop recurrent tumors. It is expected that the control animals will ultimately develop recurrent tumors, whereas treated animals either will not develop recurrent tumors or will do so at a reduced rate as compared to controls. At the end of the study (i.e., about 9-10 months after the start of treatment), the animals are sacrificed and any tumors formed are collected as described above.

Immunohistochemistry is used to evaluate the effect of the antibody on HER3 expression and phosphorylation, as well as on downstream effectors of HER3 (e.g., Akt and ERK). Paraffin-embedded tissues are sectioned and immunostained for total and phospho-HER3 (Tyr1189), phospho-HER3 (Tyr1222), phospho-HER3 (Tyr1289), phospho-Akt (Ser 473), and phospho-ERK (Thr202/Tyr204). Staining intensity in treated vs. untreated tumors is analyzed to determine whether the antibody has the desired effects. Scores are reported as percent of tumor staining positively multiplied with the intensity of staining (on a 1 to 3 scale, with 1 being weak staining intensity, 2 being moderate staining, and 3 being strong staining), resulting in a scale of 0 to 300.

The time to tumor incidence after implantation and time to tumor recurrence after castration are explored with Kaplan-Meier survival and hazard function curves (n=20 per group), and the effect of treatment on survival evaluated by log rank tests (See: Dawson-Saunders and Bat, “Methods for analyzing survival data,” Basic and Clinical Biostatistics, Appelton & Lange, Norfolk, Conn., p. 188 (1994)). Further analyses use proportional hazards models to determine the extent to which this relationship is mediated through tumor weight. The mean differences in tumor volume across the four comparison groups are assessed by analysis of variance (ANOVA). Equality of variance across the four groups is assessed using Levene's homogeneity of variance test (See: Bonham et al., J. Natl. Cancer Inst. 94(21):1641-1647 (2002)). Contrasts (each with one degree of freedom) are used to compare all treatment groups with the control group. Dunnett's multiple comparisons procedure also is used to compare all treatment groups with the control group (Bonhan et al., supra). Dunnett's procedure is used to create simultaneous (adjusted for multiple comparisons) 95% confidence intervals (CIs). P values are reported for all significance tests, and all statistical tests are two-sided. Main effects, interactions, and multiple comparisons are done, after assuring assumptions are met for a valid analysis. Standard transformations and/or the bootstrap procedure are considered if needed. Contrasts of the appropriate means can be done for specific comparisons following the analysis of variance.

### Example 6

### In Vitro Effects of U1-59 on Prostate Cancer Cell Lines

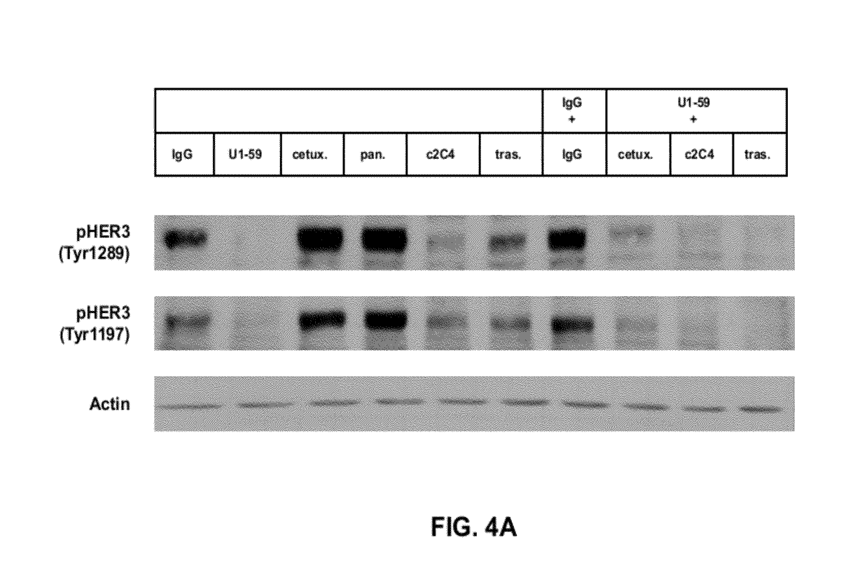

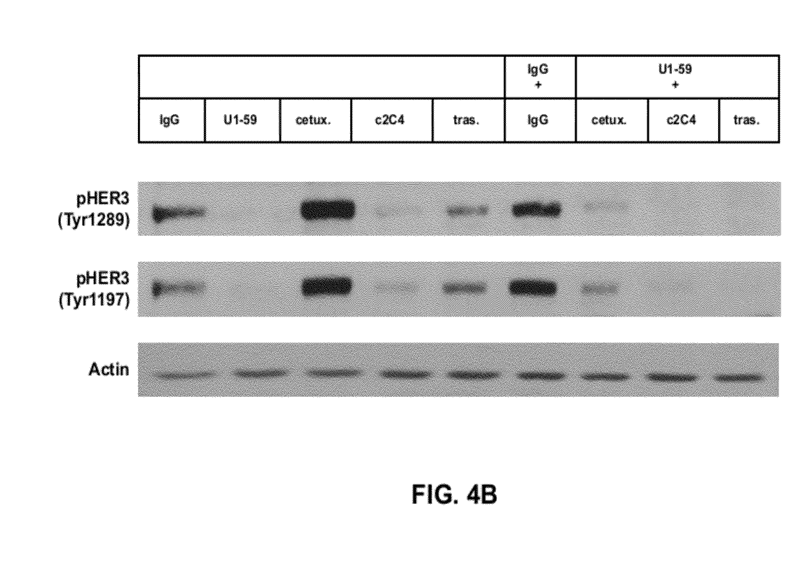

DU-145 prostate cancer cells were obtained from DSMZ (Braunschweig, Germany) and were incubated for 24 hours with IgG control, U1-59, the anti-EGF-R antibodies cetuximab and panitumumab (VECTIBIX®), the anti-HER2 antibody trastuzumab (HERCEPTIN®), the HER2 inhibitor c2C4, or combinations thereof, and effects on HER3 phosphorylation were evaluated by Western blot analysis. As shown in FIGS. 4A and 4B, western blotting of cell lysates with antibodies against particular phosphorylated tyrosine residues on HER3 (pHER3Tyr1289 and pHER3Tyr1197; Cell Signaling Technology, Beverly, Mass.)) demonstrated that U1-59 alone completely inhibit phosphorylation of HER3, while cetuximab and panitumumab increased phosphorylation, c2C4 partially inhibited phosphorylation, and trastuzumab had little effect. U1-59 in combination with cetuximab, c2C4, and trastuzumab resulted in complete or nearly complete inhibition of HER3 phosphorylation.

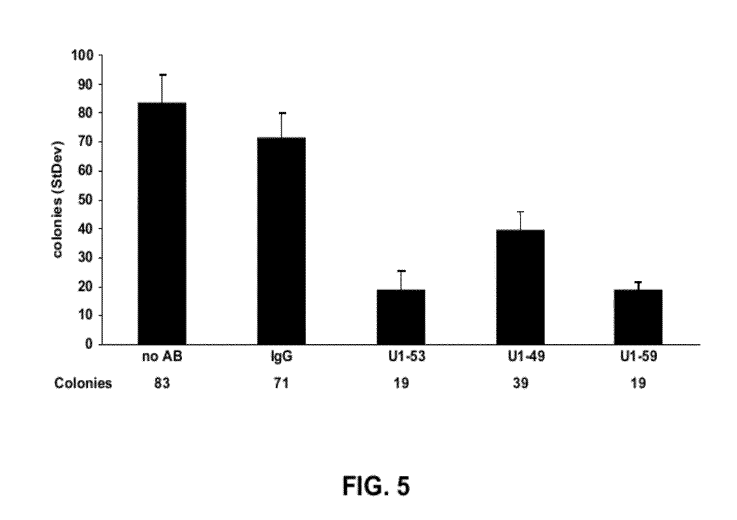

In separate experiments, the effect of U1-59 on anchorage independent growth of PC-3 prostate cancer cells, obtained from American Type Culture Collection (Manassas, Va.), was evaluated in a colony formation assay (CFA). PC-3 cells were pre-incubated with 10 g/ml each of IgG control, U1-59, or two anti-HER3 antibodies (U1-53 and U1-49) and effects on colony formation were determined in a three-layer soft agar system. The bottom agar layer contained 0.75% agar and 20% FBS in IMDM without phenol red. The top agar layer contained antibody-pre-incubated cells in 0.4% agar. The top layer was covered with a liquid feeding layer of 50 μl IMDM. Cells were cultivated at 37° C. for 12 days and colonies were counted following staining with MTT (0.21 mg/ml). As shown in FIG. 5, anchorage-independent growth of PC-3 cells was significantly inhibited by all anti-HER3 antibodies. A 78% inhibition in colony formation was obtained in the presence of U1-59 and the anti-HER3 antibody U1-53, whereas the anti-HER3 antibody U1-49 inhibited colony formation by 52%.

### Example 7

### U1-59 Inhibits Ligand-Induced HER3 Phosphorylation

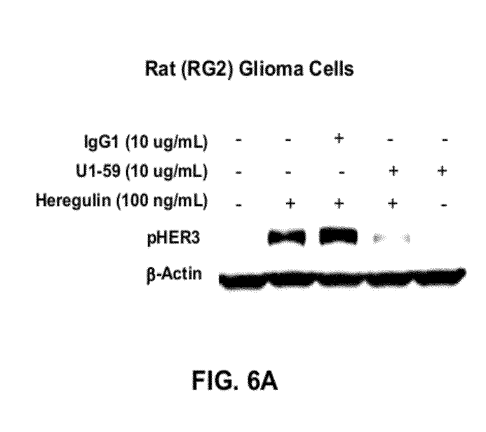

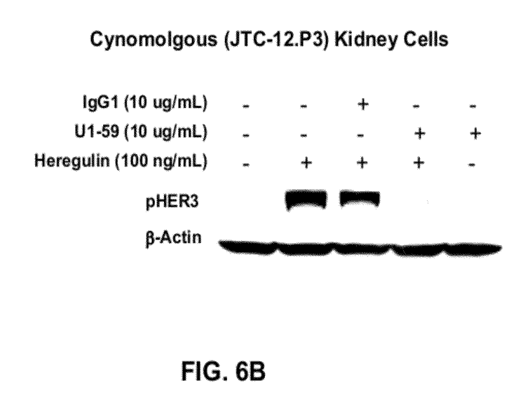

Rat RG2 glioma cells and cynomolgus monkey JTC-12.P3 kidney cells were obtained from the American Type Culture Collection (ATCC, Manassas, Va.). Cells were incubated in serum free medium with 10 g/ml IgG control or U1-59 for 1 hour prior to a 15 minute incubation with 100 ng/ml heregulin or protein control. After treatment the cells were lysed in RIPA buffer and the levels of phosphorylated HER3 were examined by western blotting (pHER3Tyr1289, Cell Signaling Technology, Beverly, Mass.). As shown in FIGS. 6A and 6B, the HER3 phosphorylation that was stimulated by heregulin was blocked by U1-59, while the IgG control had no effect.

### Example 8

### Toxicity Study of U1-59 in Rats and Monkeys

Rats (n=10/gender/dose) and cynomolgus monkeys (n=5/gender/dose) were treated once weekly for 4 weeks (five doses) with intravenous (i.v.) injection of U1-59, at doses of 20, 60, or 200 mg/kg/day. TABLE 2 shows key study findings noted after five doses and exposure levels during the fourth week of treatment. In the rats (sexually mature), the only significant finding was a reduction in prostate weight. No adverse effects were observed in either rats or monkeys. Further, there were no changes in testis weight and no microscopic changes. Statistically significant differences in prostate weights were not noted at the end of a 3-month treatment-free phase, indicating that the effects in rats were reversible.

TABLE 2
Toxicity Study Key Findings

| | Dose | AUC0-tau^{a} | |
|---|---|---|---|
| Species | (mg/kg/day) | (ug-hr/mL) | Key Findings |
| Rat | 20 | 2070 | — |
| | (NOEL) | | |
| | 60 | 4800 | ↓ mean prostate weight |
| | | | (16% mean decrease) |
| | 200 | 10300 | ↓ mean prostate weight |
| | (NOAEL) | | (22% mean decrease) |
| Cyno | 20 | 2180^{b} | — |
| | 60 | 5900 | — |
| | 200 | 13600 | — |
| | (NOEL; NOAEL) | | |

^{a}AUC₀₋ₜₐᵤ = Area under the serum concentration time curve during the final dosing interval beginning on Day 22
^{b}Two animals that tested positive for anti-U1-59 antibodies were excluded from group mean calculation.
NOEL, no observed effect level; NOAEL, no observed adverse effect level.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating benign prostate hyperplasia (BPH) in a subject in need thereof, comprising administering to the subject a therapeutically effective amount of a composition comprising a HER3 binding agent.

2. The method of claim 1, wherein the HER3 binding agent is a small molecule compound or an antigen-binding protein that binds to HER3.

3. The method of claim 1, wherein the HER3 binding agent is an antigen-binding protein that binds to HER3, and comprises:
a heavy chain amino acid sequence that comprises a CDRH1 selected from the group consisting of SEQ ID NOs:236, 251, 252, and 256; a CDRH2 selected from the group consisting of SEQ ID NOs:258, 278, 280, and 282; and a CDRH3 selected from the group consisting of SEQ ID NOs:283, 285, 309, 313, and 315; and
a light chain amino acid sequence that comprises a CDRL1 selected from the group consisting of SEQ ID NOs:320, 334, 337, and 340; a CDRL2 selected from the group consisting of SEQ ID NOs: 343, 356, 351, and 344; and a CDRL3 selected from the group consisting of SEQ ID NOs:360, 381, 385, and 387.

4. The method of claim 1, wherein the HER3 binding agent is an antigen-binding protein that binds to HER3, and comprises a heavy chain amino acid sequence that comprises at least one of the CDR's selected from the group consisting of (a) CDRH1's as shown in SEQ ID NOs:236, 251, 252, and 256; (b) CDRH2's as shown in SEQ ID NOs:258, 278, 280, and 282; and (c) CDRH3's as shown in SEQ ID NOs:283, 285, 309, 313, and 315.

5. The method of claim 1, wherein the HER3 binding agent is an antigen-binding protein that binds to HER3, and comprises a light chain amino acid sequence that comprises at least one of the CDR's selected from the group consisting of: (d) CDRL1's as shown in SEQ ID NOs: 320, 334, 337, and 340; (e) CDRL2's as shown in SEQ ID NOs:343, 356, 351, and 344; and (f) CDRL3's as shown in SEQ ID NOs:360, 381, 385, and 387.

6. The method of claim 1, wherein the HER3 binding agent is an antigen-binding protein that binds to HER3, and comprises a heavy chain amino acid sequence that comprises at least one of the CDR's selected from the group consisting of (a) CDRH1's as shown in SEQ ID NOs: 236, 251, 252, and 256; (b) CDRH2's as shown in SEQ ID NOs:258, 278, 280, and 282; and (c) CDRH3's as shown in SEQ ID NOs:283, 285, 309, 313, and 315; and a light chain amino acid sequence that comprises at least one of the CDR's selected from the group consisting of: (d) CDRL1's as shown in SEQ ID NOs:320, 334, 337, and 340; (e) CDRL2's as shown in SEQ ID NOs:343, 356, 351, and 344; and (f) CDRL3's as shown in SEQ ID NOs:360, 381, 385, and 387.

7. The method of claim 1, wherein the HER3 binding agent is an antigen-binding protein that binds to HER3, and comprises a heavy chain amino acid sequence that comprises a CDRH1 selected from the group consisting of SEQ ID NOs: 236, 251, 252, and 256, a CDRH2 selected from the group consisting of SEQ ID NOs: 258, 278, 280, and 282, and a CDRH3 selected from the group consisting of SEQ ID NOs: 283, 285, 309, 313, and 315, or a light chain amino acid sequence that comprises a CDRL1 selected from the group consisting of SEQ ID NOs: 320, 334, 337, and 340, a CDRL2 selected from the group consisting of SEQ ID NOs: 343, 356, 351, and 344, and a CDRL3 selected from the group consisting of SEQ ID NOs: 360, 381, 385, and 387.

8. The method of claim 1, wherein the HER3 binding agent is an antigen-binding protein that binds to HER3, and comprises a heavy chain amino acid sequence selected from the group consisting of SEQ ID NOs: 42, 54, 70, 92, and 96.

9. The method of claim 8, wherein the antigen-binding protein comprises a light chain amino acid sequence selected from the group consisting of SEQ ID NOs: 44, 56, 72, 94, and 98.

10. The method of claim 1, wherein the HER3 binding agent is an antigen-binding protein that binds to HER3, and comprises a heavy chain amino acid sequence selected from the group consisting of SEQ ID NOs: 42, 54, 70, 92, and 96; and a light chain amino acid sequence selected from the group consisting of SEQ ID NOs: 44, 56, 72, 94, and 98.

11. The method of claim 1, wherein the HER3 binding agent is an antigen-binding protein that binds to HER3, and comprises the heavy chain amino acid sequence of SEQ ID NO: 42 and the light chain amino acid sequence of SEQ ID NO: 44.

12. The method of claim 1, wherein the HER3 binding agent is an antigen-binding protein that binds to HER3, and comprises the heavy chain amino acid sequence of SEQ ID NO: 54 and the light chain amino acid sequence of SEQ ID NO: 56.

13. The method of claim 1, wherein the HER3 binding agent is an antigen-binding protein that binds to HER3, and comprises the heavy chain amino acid sequence of SEQ ID NO:70 and the light chain amino acid sequence of SEQ ID NO: 72.

14. The method of claim 1, wherein the HER3 binding agent is an antigen-binding protein that binds to HER3, and comprises a CDRH3 selected from the group consisting of SEQ ID NOs: 283, 285, 309, 313, and 315.

15. The method of claim 1, wherein the HER3 binding agent is an antigen-binding protein that binds to HER3, and comprises a CDHL3 selected from the group consisting of SEQ ID NOs: 360, 381, 385, and 387.

16. The method of claim 3, wherein the antigen-binding protein is directed against the extracellular domain of HER3.

17. The method of claim 3, wherein binding of the antigen-binding protein to HER3 has one or more effects selected from the group consisting of reduction of HER3-mediated signal transduction, reduction of HER3 phosphorylation, reduction of cell proliferation, reduction of cell migration, and increasing downregulation of HER3.

18. The method of claim 3, wherein the antigen-binding protein that binds to HER3 is an antibody.

19. The method of claim 18, wherein the antibody is a monoclonal antibody, a polyclonal antibody, a recombinant antibody, a humanized antibody, a human antibody, a chimeric antibody, a multispecific antibody, or an antibody fragment thereof.

20. The method of claim 19, wherein the antibody fragment is a Fab fragment, a Fab′ fragment, a F(ab′)2 fragment, a Fv fragment, a diabody, or a single chain antibody molecule.

21. The method of claim 18, wherein the antibody is of the IgG1-, IgG2-, IgG3- or IgG4-type.

22. The method of claim 1, wherein the HER3 binding agent is an antigen-binding protein that binds to HER3, and wherein the antigen-binding protein is coupled to an effector group.

23. The method of claim 22, wherein the effector group is a radioisotope or radionuclide, a toxin, or a therapeutic or chemotherapeutic group.

24. The method of claim 23, wherein the therapeutic or chemotherapeutic group is selected from the group consisting of calicheamicin, auristatin-PE, geldanamycin, maytansine and derivatives thereof.

25. The method of claim 1, further comprising identifying the subject as having BPH.

26. The method of claim 1, further comprising monitoring prostate size in the subject after administering the composition.

27. A method for reducing prostate weight in a subject, comprising administering to the subject a therapeutically effective amount of a composition comprising a HER3 binding agent.

28. The method of claim 27, wherein the HER3 binding agent is a small molecule compound or an antigen-binding protein that binds to HER3.

29. The method of claim 27, wherein the HER3 binding agent is an antigen-binding protein that binds to HER3, and comprises:
a heavy chain amino acid sequence that comprises a CDRH1 selected from the group consisting of SEQ ID NOs: 236, 251, 252, and 256; a CDRH2 selected from the group consisting of SEQ ID NOs: 258, 278, 280, and 282; and a CDRH3 selected from the group consisting of SEQ ID NOs: 283, 285, 309, 313, and 315; and
a light chain amino acid sequence that comprises a CDRL1 selected from the group consisting of SEQ ID NOs: 320, 334, 337, and 340; a CDRL2 selected from the group consisting of SEQ ID NOs: 343, 356, 351, and 344; and a CDRL3 selected from the group consisting of SEQ ID NOs: 360, 381, 385, and 387.


SEQUENCE LISTING (390 sequences)

SEQ ID NO: 1 - DNA, 342 bp, Artificial Sequence
  Description of Artificial Sequence Synthetic polynucleotide construct
gaggtgcagctggtggagtctggaggaggcttgatccagcctggggggtccctgagactctcctgtgcagcctctgggttcaccgtcagtagcaactacatgagctgggtccgccaggctccagggaaggggctggattgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagggcagtggctggacgtctggggccaagggaccacggtcaccgtctcctca

SEQ ID NO: 2 - protein, 114 aa, Artificial Sequence
  Description of Artificial Sequence Synthetic polypeptide construct
EVQLVESGGGLIQPGGSLRLSCAASGFTVSSNYMSWVRQAPGKGLDWVSVIYSGGSTYYADSVKGRFTISRDNSKNTLYLQMNSLRAEDTAVYYCARGQWLDVWGQGTTVTVSS

SEQ ID NO: 3 - DNA, 336 bp, Artificial Sequence
  Description of Artificial Sequence Synthetic polynucleotide construct
gatattgtgatgactcagtctccactctccctgcccgtcacccctggagagccggcctccatctcctgcaggtcaagtcagagcctcctgcatagtaatggatacaactatttggattggtacctgcagaggccagggcagtctccacaactcctgttctatttgggttttcatcgggcctccggggtccctgacaggttcagtggcagtggatcaggcacagattttacactgaaaatcagcagagtggaggctgaggatgttggggtttattactgcaggcaagctctacaaactccgctcactttcggcggagggaccaaggtggagatcaaa

SEQ ID NO: 4 - protein, 112 aa, Artificial Sequence
  Description of Artificial Sequence Synthetic polypeptide construct
DIVMTQSPLSLPVTPGEPASISCRSSQSLLHSNGYNYLDWYLQRPGQSPQLLFYLGFHRASGVPDRFSGSGSGTDFTLKISRVEAEDVGVYYCRQALQTPLTFGGGTKVEIK

SEQ ID NO: 5 - DNA, 374 bp, Artificial Sequence
  Description of Artificial Sequence Synthetic polynucleotide construct
caggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattccagtgggagcacctactacaacccgtccctcaagagtcgagttaccatatcagtagacacgtctaagaaccagttctccctgaagctgagctctgtgactgccgcggacacggccgtgtattactgtgcgagagatagggaactggaactttactactactactacggtatggacgtctggggccaagggaccacggtcaccgtctcctc

SEQ ID NO: 6 - protein, 124 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYSSGSTYYNPSLKSRVTISVDTSKNQFSLKLSSVTAADTAVYYCARDRELELYYYYYGMDVWGQGTTVTVS

SEQ ID NO: 7 - DNA, 336 bp, Artificial Sequence
  Description of Artificial Sequence Synthetic polynucleotide construct
gatattgtgatgactcagtctccactctccctgcccgtcacccctggagagccggcctccatctcctgcaggtctagtcagagcctcctgtatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttgggatttattactgcatgcaagctctacaaactccgctcactttcggcggagggaccaaggtggagatcaaa

SEQ ID NO: 8 - protein, 112 aa, Artificial Sequence
  Description of Artificial Sequence Synthetic polypeptide construct
DIVMTQSPLSLPVTPGEPASISCRSSQSLLYSNGYNYLDWYLQKPGQSPQLLIYLGSNRASGVPDRFSGSGSGTDFTLKISRVEAEDVGIYYCMQALQTPLTFGGGTKVEIK

SEQ ID NO: 9 - DNA, 357 bp, Artificial Sequence
  Description of Artificial Sequence Synthetic polynucleotide construct
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggactggcttgcactcatttattggaatgatgataagcgctacagcccatctctgaagagcaggctcaccatcaccaaggacacctccaaaaaccaggtggtccttacaatgaccaacatggatcttgtggacacagccacatattactgtgtacacagagacgaagttcgagggtttgactactggggccagggaaccctggtcaccgtctcctca

SEQ ID NO: 10 - protein, 119 aa, Artificial Sequence
  Description of Artificial Sequence Synthetic polypeptide construct
QITLKESGPTLVKPTQTLTLTCTFSGFSLSTSGVGVGWIRQPPGKALDWLALIYWNDDKRYSPSLKSRLTITKDTSKNQVVLTMTNMDLVDTATYYCVHRDEVRGFDYWGQGTLVTVSS

SEQ ID NO: 11 - DNA, 336 bp, Artificial Sequence
  Description of Artificial Sequence Synthetic polynucleotide construct
gatgttgtgatgactcagtctccactctccctgcccgtcacccttggacagccggcctccatctcctgcaggtctagtcaaagcctcgtatacagtgatggatacacctacttgcattggtttcagcagaggccaggccaatctccaaggcgccttatttataaggtttctaactgggactctggggtcccagacagattcagcggcagtgggtcaggcactgatttcacactgaaaatcagcagggtggaggctgaggatgttggggtttattactgcatgcaaggtgcacactggccgatcaccttcggccaagggacacgactggagattaaa

SEQ ID NO: 12 - protein, 112 aa, Artificial Sequence
  Description of Artificial Sequence Synthetic polypeptide construct
DVVMTQSPLSLPVTLGQPASISCRSSQSLVYSDGYTYLHWFQQRPGQSPRRLIYKVSNWDSGVPDRFSGSGSGTDFTLKISRVEAEDVGVYYCMQGAHWPITFGQGTRLEIK

SEQ ID NO: 13 - DNA, 374 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgggtactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtatttctgtgcgagagatcgggaacttgagggttactccaactactacggtgtggacgtctggggccaagggaccacggtcaccgtctcctc

SEQ ID NO: 14 - protein, 124 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYFCARDRELEGYSNYYGVDVWGQGTTVTVS

SEQ ID NO: 15 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcaggccattagcaactatttaaattggtatcagcagaaaccagggaaagcccctaagctcctgatctatgctgcatccagtttgcaaagtggggtcccatcaaggttcagtggcagtggatctgggacagatttcactctcaccatcagcagcctgcagcctgaagattttgcaacttattactgtcaacagaataatagtctcccgatcaccttcggccaagggacacgactggagattaaa

SEQ ID NO: 16 - protein, 107 aa, Artificial Sequence
  Description of Artificial Sequence Synthetic polypeptide construct
DIQMTQSPSSLSASVGDRVTITCRASQAISNYLNWYQQKPGKAPKLLIYAASSLQSGVPSRFSGSGSGTDFTLTISSLQPEDFATYYCQQNNSLPITFGQGTRLEIK

SEQ ID NO: 17 - DNA, 354 bp, Artificial Sequence
  Description of Artificial Sequence Synthetic polynucleotide construct
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatgaaaactacggtgactacaactactggggccagggaaccctggtcaccgtctcctca

SEQ ID NO: 18 - protein, 118 aa, Artificial Sequence
  Description of Artificial Sequence Synthetic polypeptide construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHENYGDYNYWGQGTLVTVSS

SEQ ID NO: 19 - DNA, 321 bp, Artificial Sequence
  Description of Artificial Sequence Synthetic polynucleotide construct
gacatccagatgacccagtctccatcctccctgtctgcatctgtcggagacagagtcaccatcacttgccgggcaagtcagagcattcgcagctatttaaattggtatcagcagaaaccagggaaagcccctaagctcctgatctatgctgcttccagtttgcaaagtggggtcccatcaaggttcagtggcagtggatctgggacagatttcactctcaccatcagcagtctgcaacctgaagattttgcactttactgctgtcaacagagtaacggttccccgctcactttcggcggagggaccaaggtggagatcaaa

SEQ ID NO: 20 - protein, 107 aa, Artificial Sequence
  Description of Artificial Sequence Synthetic polypeptide construct
DIQMTQSPSSLSASVGDRVTITCRASQSIRSYLNWYQQKPGKAPKLLIYAASSLQSGVPSRFSGSGSGTDFTLTISSLQPEDFALYCCQQSNGSPLTFGGGTKVEIK

SEQ ID NO: 21 - DNA, 380 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaggagtcgagttaccatatcagtagacacgtctaagaaccagttctccctgaagctgagctctgtgactgccgcggacacggccgtgtattactgtgcgagagatagagagagagagtgggatgattacggtgacccccaaggtatggacgtctggggccaagggaccacggtcaccgtctcctc

SEQ ID NO: 22 - protein, 126 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYYSGSTYYNPSLRSRVTISVDTSKNQFSLKLSSVTAADTAVYYCARDREREWDDYGDPQGMDVWGQGTTVTVS

SEQ ID NO: 23 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagagcattagcagctatttacattggtatcagcagaaaccagggaaagcccctaagctcctgatccatgctgcatccagtttacaaagtggggtcccatcaaggttcagtggcagtggatctgggacagatttcactctcaccatcagtagtctgcaacctgaagattttgcaacttactactgtcaacagagttacagtaacccgctcactttcggcggagggaccaaggtggagatccaa

SEQ ID NO: 24 - protein, 107 aa, Artificial Sequence
  Description of Artificial Sequence Synthetic polypeptide construct
DIQMTQSPSSLSASVGDRVTITCRASQSISSYLHWYQQKPGKAPKLLIHAASSLQSGVPSRFSGSGSGTDFTLTISSLQPEDFATYYCQQSYSNPLTFGGGTKVEIQ

SEQ ID NO: 25 - DNA, 354 bp, Artificial Sequence
  Description of Artificial Sequence Synthetic polynucleotide construct
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctggcctggtgactctgataccatatacagcccgtccttccaaggccaggtcaccatctcagccgacaagtccatcagcaccgcctacctgcagtggagcagcctgaaggcctcggacaccgccatgtattactgtgcgagacatgaaaactacggtgactacaactactggggccagggaaccctggtcaccgtctcctca

SEQ ID NO: 26 - protein, 118 aa, Artificial Sequence
  Description of Artificial Sequence Synthetic polypeptide construct
EVQLVQSGAEVKKPGESLKISCKGSGYSFTSYWIGWVRQMPGKGLEWMGIIWPGDSDTIYSPSFQGQVTISADKSISTAYLQWSSLKASDTAMYYCARHENYGDYNYWGQGTLVTVSS

SEQ ID NO: 27 - DNA, 321 bp, Artificial Sequence
  Description of Artificial Sequence Synthetic polynucleotide construct
gacatccagatgacccagtctccatcctccctgtctgcatctgtgggagacagagtcaccatcacttgccgggcaagtcagagcattcgaagttatttaaattggtatcagcagaaaccggggaatgcccctaaactcctgatctatgctgcatccagtttgcaaagtggggtcccatcaaggttcagtggcagtggatctgggacagatttcactctcaccatcagcagtctgcaacctgaagattttgcactttactactgtcaacagagtatcagttccccgctcactttcggcggagggaccaaggtggagatcaaa

SEQ ID NO: 28 - protein, 107 aa, Artificial Sequence
  Description of Artificial Sequence Synthetic polypeptide construct
DIQMTQSPSSLSASVGDRVTITCRASQSIRSYLNWYQQKPGNAPKLLIYAASSLQSGVPSRFSGSGSGTDFTLTISSLQPEDFALYYCQQSISSPLTFGGGTKVEIK

SEQ ID NO: 29 - DNA, 374 bp, Artificial Sequence
  Description of Artificial Sequence Synthetic polynucleotide construct
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtgacactggctatgcacaggtgttccagggcagagtcaccatgacctggaacacctccataagcacagcctacatggaactgagcagcctgagatctgaggacacggccgtgtattactgtgcgagatttggggatctcccgtatgactacagttactacgaatggttcgacccctggggccagggaaccctggtcaccgtctcctc

SEQ ID NO: 30 - protein, 124 aa, Artificial Sequence
  Description of Artificial Sequence Synthetic polypeptide construct
QVQLVQSGAEVKKPGASVKVSCKASGYTFTSYDINWVRQATGQGLEWMGWMNPNSGDTGYAQVFQGRVTMTWNTSISTAYMELSSLRSEDTAVYYCARFGDLPYDYSYYEWFDPWGQGTLVTVS

SEQ ID NO: 31 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagccagagcattagcagctatttaaattggtatcagcagagaccagggaaagcccctaagctcctgatctatgcagcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaa

SEQ ID NO: 32 - protein, 107 aa, Artificial Sequence
  Description of Artificial Sequence Synthetic polypeptide construct
DIQMTQSPSSLSASVGDRVTITCRASQSISSYLNWYQQRPGKAPKLLIYAASSLQSGVPSRFSGSGSGTDFTLTISSLQPEDFATYYCQQSYSTPLTFGGGTKVEIK

SEQ ID NO: 33 - DNA, 386 bp, Artificial Sequence
  Description of Artificial Sequence Synthetic polynucleotide construct
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagatctctacgatttttggagtggttatccctactactacggtatggacgtctggggccaagggaccacggtcaccgtctcctc

SEQ ID NO: 34 - protein, 128 aa, Artificial Sequence
  Description of Artificial Sequence Synthetic polypeptide construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDLYDFWSGYPYYYGMDVWGQGTTVTVS

SEQ ID NO: 35 - DNA, 383 bp, Artificial Sequence
  Description of Artificial Sequence Synthetic polynucleotide construct
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagattactatggttcggggagtttctactactactacggtatggacgtctggggccaagggaccacggtcaccgtctcctc

SEQ ID NO: 36 - protein, 127 aa, Artificial Sequence
  Description of Artificial Sequence Synthetic polypeptide construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDYYGSGSFYYYYGMDVWGQGTTVTVS

SEQ ID NO: 37 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagagcattagcagctatttaaattggtatcagcagaaaccagggaaagcccctaaggtcctgatctatgctgcatccaatttgcaaagtggggtcccatcaaggttcagtggcagtggatctgggacagatttcactctcaccatcagcagtctgcaacctgaagattttgcaacttactactgtcaacagagttacagtacccctcggacgttcggccaagggaccaaggtggaaatcaaa

SEQ ID NO: 38 - protein, 107 aa, Artificial Sequence
  Description of Artificial Sequence Synthetic polypeptide construct
DIQMTQSPSSLSASVGDRVTITCRASQSISSYLNWYQQKPGKAPKVLIYAASNLQSGVPSRFSGSGSGTDFTLTISSLQPEDFATYYCQQSYSTPRTFGQGTKVEIK

SEQ ID NO: 39 - DNA, 371 bp, Artificial Sequence
  Description of Artificial Sequence Synthetic polynucleotide construct
caggtgcagctgcaggagtcgggcccaggactggtgaagccttcggagaccctgtccctcacctgcactgtctctggtggctccatcagtagttactactggagctggatccggcagcccgccgggaagggactggagtggattgggcatatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagaagcgatttttggagtgggcccctactactactacggtatggacgtctggggccaagggaccacggtcaccgtctcctc

SEQ ID NO: 40 - protein, 123 aa, Artificial Sequence
  Description of Artificial Sequence Synthetic polypeptide construct
QVQLQESGPGLVKPSETLSLTCTVSGGSISSYYWSWIRQPAGKGLEWIGHIYTSGSTNYNPSLKSRVTMSVDTSKNQFSLKLSSVTAADTAVYYCAREAIFGVGPYYYYGMDVWGQGTTVTVS

SEQ ID NO: 41 - DNA, 377 bp, Artificial Sequence
  Description of Artificial Sequence Synthetic polynucleotide construct
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaatattggtggcacaaactgtgcacagaagtttcagggcagggtcaccatgaccagggacacgtccatcagcacagcctacatggagctgagcaggctgagatctgacgacacggccgtgtattactgtgcgagagggggacggtatagcagcagctggtcctactactactacggtatggacgtctggggccaagggaccacggtcaccgtctcctc

SEQ ID NO: 42 - protein, 125 aa, Artificial Sequence
  Description of Artificial Sequence Synthetic polypeptide construct
QVQLVQSGAEVKKPGASVKVSCKASGYTFTGYYMHWVRQAPGQGLEWMGWINPNIGGTNCAQKFQGRVTMTRDTSISTAYMELSRLRSDDTAVYYCARGGRYSSSWSYYYYGMDVWGQGTTVTVS

SEQ ID NO: 43 - DNA, 336 bp, Artificial Sequence
  Description of Artificial Sequence Synthetic polynucleotide construct
gatattctgatgacccagactccactctctctgtccgtcacccctggacagccggcctccatctcctgcaagtctagtcagagcctcctgcttagtgatggagggacctatttgtattggtacctgcagaagccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatgcagcttccgatcaccttcggccaagggacacgactggaaattaaa

SEQ ID NO: 44 - protein, 112 aa, Artificial Sequence
  Description of Artificial Sequence Synthetic polypeptide construct
DILMTQTPLSLSVTPGQPASISCKSSQSLLLSDGGTYLYWYLQKPGQPPQLLIYEVSNRFSGVPDRFSGSGSGTDFTLKISRVEAEDVGVYYCMQSMQLPITFGQGTRLEIK

SEQ ID NO: 45 - DNA, 380 bp, Artificial Sequence
  Description of Artificial Sequence Synthetic polynucleotide construct
caggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagggggggacagtaactacgaggattactactactactacggtatggacgtctggggccaagggaccacggtcaccgtctcctc

SEQ ID NO: 46 - protein, 126 aa, Artificial Sequence
  Description of Artificial Sequence Synthetic polypeptide construct
QVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCARGGDSNYEDYYYYYGMDVWGQGTTVTVS

SEQ ID NO: 47 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagagcattagcatctatttacattggtatcagcagaaaccagggaaagcccctaagctcttgatctctgctgcatccagtttgcaaagtggggtcccgtcaaggttcagtggcagtggatctgggacagatttcactctcaccatcagaagtctgcaacctgaagattttgcaacttactactgtcaacagagttacacttccccgatcaccttcggccaagggacacgactggagattaaa

SEQ ID NO: 48 - protein, 107 aa, Artificial Sequence
  Description of Artificial Sequence Synthetic polypeptide construct
DIQMTQSPSSLSASVGDRVTITCRASQSISIYLHWYQQKPGKAPKLLISAASSLQSGVPSRFSGSGSGTDFTLTIRSLQPEDFATYYCQQSYTSPITFGQGTRLEIK

SEQ ID NO: 49 - DNA, 380 bp, Artificial Sequence
  Description of Artificial Sequence Synthetic polynucleotide construct
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagcaccagttctccctgaagctgagctctgtgaccgctgcggacacggccgtgtattactgtgcgagagattcgagttactatgatagtagtggttattacttatactactacgctatggacgtctggggccaagggaccacggtcaccgtctcctc

SEQ ID NO: 50 - protein, 126 aa, Artificial Sequence
  Description of Artificial Sequence Synthetic polypeptide construct
QVQLQESGPGLVKPSETLSLTCTVSGGSISSYYWSWIRQPPGKGLEWIGYIYYSGSTNYNPSLKSRVTISVDTSKHQFSLKLSSVTAADTAVYYCARDSSYYDSSGYYLYYYAMDVWGQGTTVTVS

SEQ ID NO: 51 - DNA, 339 bp, Artificial Sequence
  Description of Artificial Sequence Synthetic polynucleotide construct
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttcctgggcatctacccgggaatccggggtccctgaccgattcagtggcagcgggtctgggacagatttcactctcaccatcagcagcctgcaggctgaagatgtggcagtttattactgtcagcaatattatactactcctctcactttcggccctgggaccaaagtggatatcaaa

SEQ ID NO: 52 - protein, 113 aa, Artificial Sequence
  Description of Artificial Sequence Synthetic polypeptide construct
DIVMTQSPDSLAVSLGERATINCKSSQSVLYSSNNKNYLAWYQQKPGQPPKLLISWASTRESGVPDRFSGSGSGTDFTLTISSLQAEDVAVYYCQQYYTTPLTFGPGTKVDIK

SEQ ID NO: 53 - DNA, 357 bp, Artificial Sequence
  Description of Artificial Sequence Synthetic polynucleotide construct
gaggtgcaactggtggagtctgggggaggcttggtacagcctggggggtccctgagactctcctgtgcagcctctggattcaccttcagtatctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagacgaggacacggctgtgtattactgtgcgagagataggggtgacttcgatgcttttgatatctggggccaagggacaatggtcaccgtctcttca

SEQ ID NO: 54 - protein, 119 aa, Artificial Sequence
  Description of Artificial Sequence Synthetic polypeptide construct
EVQLVESGGGLVQPGGSLRLSCAASGFTFSIYSMNWVRQAPGKGLEWVSYISSSSSTIYYADSVKGRFTISRDNAKNSLYLQMNSLRDEDTAVYYCARDRGDFDAFDIWGQGTMVTVSS

SEQ ID NO: 55 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccaggcgagtcaggacattaccaactatttgaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatataactgtcaacagtgtgaaaatttcccgatcaccttcggccaagggacacgactggagattaaa

SEQ ID NO: 56 - protein, 107 aa, Artificial Sequence
  Description of Artificial Sequence Synthetic polypeptide construct
DIQMTQSPSSLSASVGDRVTITCQASQDITNYLNWYQQKPGKAPKLLIYDASNLETGVPSRFSGSGSGTDFTFTISSLQPEDIATYNCQQCENFPITFGQGTRLEIK

SEQ ID NO: 57 - DNA, 336 bp, Artificial Sequence
  Description of Artificial Sequence Synthetic polynucleotide construct
gatattgtgatgactcagtctccactctccctgcccgtcacccctggagagccggcctccatctcctgcaggtctagtcagagcctcctgtatagtaatggatacaagtatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattattgcatgcaggctctacaaactccgatcaccttcggccaagggacacgactggagattaaa

SEQ ID NO: 58 - protein, 112 aa, Artificial Sequence
  Description of Artificial Sequence Synthetic polypeptide construct
DIVMTQSPLSLPVTPGEPASISCRSSQSLLYSNGYKYLDWYLQKPGQSPQLLIYLGSNRASGVPDRFSGSGSGTDFTLKISRVEAEDVGVYYCMQALQTPITFGQGTRLEIK

SEQ ID NO: 59 - DNA, 374 bp, Artificial Sequence
  Description of Artificial Sequence Synthetic polynucleotide construct
caggtgcagctgcaggagtcgggcccaggactggtgaagccttcggagaccctgtccctcacctgcactgtctctggtggctccgtcagcagtggtggttactactggaactggatccggcagcccccagggaagggactggagtggattgggtatatcaattacagtgggagcaccaactacaacccctccctcaagagtcgagtcaccatatcagtagacacgtccaagaaccagttctccctgaagctgagctctgtgaccgctgcggacacggccgtgtattactgtgcgagagatcgagaactggaactttactactactactacggtatggacgtctggggccaagggaccacggtcaccgtctcctc

SEQ ID NO: 60 - protein, 124 aa, Artificial Sequence
  Description of Artificial Sequence Synthetic polypeptide construct
QVQLQESGPGLVKPSETLSLTCTVSGGSVSSGGYYWNWIRQPPGKGLEWIGYINYSGSTNYNPSLKSRVTISVDTSKNQFSLKLSSVTAADTAVYYCARDRELELYYYYYGMDVWGQGTTVTVS

SEQ ID NO: 61 - DNA, 374 bp, Artificial Sequence
  Description of Artificial Sequence Synthetic polynucleotide construct
caggtgcagctgcaggagtcgggcccaggactggtgaagccttctgagaccctgtccctcacctgcactgtctctggtggctccgtcagcagtggtggttactactggaactggatccggcagcccccagggaagggactggagtggattgggtatatcaattacagtgggagcaccaactacaacccctccctcaagagtcgagtcaccatatcagtagacacgtccaagaaccagttctccctgaagctgagctctgtgaccgctgcggacacggccgtgtattactgtgcgagagatcgagaactggaactttactactactactacggtatggacgtctggggccaagggaccacggtcaccgtctcctc

SEQ ID NO: 62 - protein, 124 aa, Artificial Sequence; identical to SEQ ID NO: 60
  Description of Artificial Sequence Synthetic polypeptide construct

SEQ ID NO: 63 - DNA, 336 bp, Artificial Sequence
  Description of Artificial Sequence Synthetic polynucleotide construct
gatattgtgatgactcagtctccactctccctgcccgtcacccctggagagccggcctccatctcctgcaggtctagtcagagcctcctgtatagtaatggatacaagtatttggattggtacctgcagaagccagggcagtctccacagctcatgatctatttgggttctaatcgggcctccggggtccctgacaggttcagtggcagtggatcaggcacagattttacactgaaaatcagcagagtggaggctgaggatgttggggtttattattgcatgcaggctctacaaactccgatcaccttcggccaagggacacgactggagattaaa

SEQ ID NO: 64 - protein, 112 aa, Artificial Sequence
  Description of Artificial Sequence Synthetic polypeptide construct
DIVMTQSPLSLPVTPGEPASISCRSSQSLLYSNGYKYLDWYLQKPGQSPQLMIYLGSNRASGVPDRFSGSGSGTDFTLKISRVEAEDVGVYYCMQALQTPITFGQGTRLEIK

SEQ ID NO: 65 - DNA, 363 bp, Artificial Sequence
  Description of Artificial Sequence Synthetic polynucleotide construct
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagcagctcgccttgactactactacggtatggacgtctggggccaagggaccacggtcaccgtctcctca

SEQ ID NO: 66 - protein, 121 aa, Artificial Sequence
  Description of Artificial Sequence Synthetic polypeptide construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARAARLDYYYGMDVWGQGTTVTVSS

SEQ ID NO: 67 - DNA, 321 bp, Artificial Sequence
  Description of Artificial Sequence Synthetic polynucleotide construct
gacatccagatgacccagtctccatcctccctgtctgcatctgtaggagacagagtctccatcacttgccgggcaagtcagagcattaacagctatttaaattggtttcagcagaagccagggaaagcccctcagctcctgatctttggtgcatccggtttgcaaagtggggtcccatcaaggttcagtggcagtggatctgggacagatttcactctcaccatcaacagtctgcaacctgaagattttgcaacttactactgtcaacagagttacagttccccgctcaccttcggccaagggacacgactggagattaaa

SEQ ID NO: 68 - protein, 107 aa, Artificial Sequence
  Description of Artificial Sequence Synthetic polypeptide construct
DIQMTQSPSSLSASVGDRVSITCRASQSINSYLNWFQQKPGKAPQLLIFGASGLQSGVPSRFSGSGSGTDFTLTINSLQPEDFATYYCQQSYSSPLTFGQGTRLEIK

SEQ ID NO: 69 - DNA, 351 bp, Artificial Sequence
  Description of Artificial Sequence Synthetic polynucleotide construct
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagaaacgtccaagaaccagttctccctgaagctgagctctgtgaccgccgcggacacggctgtgtattactgtgcgagagataagtggacctggtacttcgatctctggggccgtggcaccctggtcactgtctcctca

SEQ ID NO: 70 - protein, 117 aa, Artificial Sequence [sequence omitted]

SEQ ID NO: 71 - DNA, 339 bp, Artificial Sequence
  Description of Artificial Sequence Synthetic polynucleotide construct
gacatcgagatgacccagtctccagactccctggctgtgtctctgggcgagagggccaccatcaactgcaggtccagccagagtgttttatacagctccagcaataggaactacttagcttggtaccagcagaacccaggacagcctcctaagctgctcatttactgggcttctacccgggaatccggggtccctgaccgattcagtggcagcgggtctgggacagatttcactctcaccatcagcagcctgcaggctgaagatgtggcagtttattactgtcagcaatattatagtactcctcggacgttcggccaagggaccaaggtggaaatcaaa

SEQ ID NO: 72 - protein, 113 aa, Artificial Sequence [sequence omitted]

SEQ ID NO: 73 - DNA, 374 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtggttactactggagctggatccgccagcacccagggaagggcctggagtggatggggaacatctattacagtgggagcacctactacaacccgtccctcaagagtcgagttaccatatcagtagacacgtctgagaaccagttctccctgaagctgaactctgtgactgccgcggacacggccgtatattactgtgcgagagggggaactggaaccaattactactactactacggtatggacgtctggggccaagggaccacggtcaccgtctcctc

SEQ ID NO: 74 - protein, 124 aa, Artificial Sequence
  Description of Artificial Sequence Synthetic polypeptide construct
QVQLQESGPGLVKPSQTLSLTCTVSGGSISSGGYYWSWIRQHPGKGLEWMGNIYYSGSTYYNPSLKSRVTISVDTSENQFSLKLNSVTAADTAVYYCARGGTGTNYYYYYGMDVWGQGTTVTVS

SEQ ID NO: 75 - DNA, 324 bp, Artificial Sequence
  Description of Artificial Sequence Synthetic polynucleotide construct
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagctgggccactggcatcccaaacaggttcagtggcagtgggtctgggacagacttcactctcaccatcagcagactggagcctgaagattttgcagtgtattactgtcagcagtatggtagctcaccgctcactttcggcggagggaccaaggtggagatcaaa

SEQ ID NO: 76 - protein, 108 aa, Artificial Sequence
  Description of Artificial Sequence Synthetic polypeptide construct
EIVLTQSPGTLSLSPGERATLSCRASQSVSSSYLAWYQQKPGQAPRLLIYGASSWATGIPNRFSGSGSGTDFTLTISRLEPEDFAVYYCQQYGSSPLTFGGGTKVEIK

SEQ ID NO: 77 - DNA, 377 bp, Artificial Sequence
  Description of Artificial Sequence Synthetic polynucleotide construct
caggtgcagctgcaggagtcgggcccaggactggtgaagccttcacagaccctgtccctcacctgcactgtctctggtgtctccatcagcagtggtggttactactggagctggatccgccagcacccagggatgggcctggagtggattgggtacatctattacagtgggagcacctactacaacccgtccctcaagagtcgagtcaccatatcagaagacacgtctaagaaccagttctccctgaagctgagctctgtgactgccgcggacacggccgtgtattactgtgcgagagattccgagtccgagtatagcagctcgtcgaactacggtatggacgtctggggccaagggaccacggtcaccgtctcctc

SEQ ID NO: 78 - protein, 125 aa, Artificial Sequence
  Description of Artificial Sequence Synthetic polypeptide construct
QVQLQESGPGLVKPSQTLSLTCTVSGVSISSGGYYWSWIRQHPGMGLEWIGYIYYSGSTYYNPSLKSRVTISEDTSKNQFSLKLSSVTAADTAVYYCARDSESEYSSSSNYGMDVWGQGTTVTVS

SEQ ID NO: 79 - DNA, 377 bp, Artificial Sequence; identical to SEQ ID NO: 77
  Description of Artificial Sequence Synthetic polynucleotide construct

SEQ ID NO: 80 - protein, 125 aa, Artificial Sequence; identical to SEQ ID NO: 78
  Description of Artificial Sequence Synthetic polypeptide construct

SEQ ID NO: 81 - DNA, 321 bp, Artificial Sequence
  Description of Artificial Sequence Synthetic polynucleotide construct
gacatccagatgacccagtctccatcctccctgtctgcatctgtaggagacagaatcaccatcacttgccgggcaagtcagaccattagcagctatttaaattggtatcagcagaaaccagggaaagcccctaagctcctgatctatgctgcatccagtttgcaaggtggggtcccatcaaggttcagtggcagtgtatctgggacagatttcaccctcaccgtcagcagtctgcaacctgaagattttgcaacttactactgtcaacagagttacagtaacccgctcactttcggcggagggaccaaggtggagatcaaa

SEQ ID NO: 82 - protein, 107 aa, Artificial Sequence
  Description of Artificial Sequence Synthetic polypeptide construct
DIQMTQSPSSLSASVGDRITITCRASQTISSYLNWYQQKPGKAPKLLIYAASSLQGGVPSRFSGSVSGTDFTLTVSSLQPEDFATYYCQQSYSNPLTFGGGTKVEIK

SEQ ID NO: 83 - DNA, 371 bp, Artificial Sequence
  Description of Artificial Sequence Synthetic polynucleotide construct
gaggtgcagctggtgcagtctggagcagaggtgaaaaagcccggggagtctctgaagatctcctgtaagggttctggatacagttttaccagctactggatcggctgggtgcgccagatgcccgggaaaggcctggagtggatggggatcatctatcctggtgactctgataccagatacagcccgtccttccaaggccaggtcaccatgtcagccgacaagtccatcagtaccgcctacctgcagctgagcagccatgaaggcctcggacaccgccatgtattactgtgcgagacagatggctggaaactacgtacatcacgggtgatcgagacgtcctggggccaagggaccacggtcaccgtctcctc

SEQ ID NO: 84 - protein, 123 aa, Artificial Sequence
  Description of Artificial Sequence Synthetic polypeptide construct
EVQLVQSGAEVKKPGESLKISCKGSGYSFTSYWIGWVRQMPGKGLEWMGIIYPGDSDTRYSPSFQGQVTMSADKSISTAYLQLSSHEGLGHRHVLLCETDGWKLRTSRVIETSWGQGTTVTVS

SEQ ID NO: 85 - DNA, 324 bp, Artificial Sequence
  Description of Artificial Sequence Synthetic polynucleotide construct
gaaattgtgttgacgcagtctccaggcaccctgtctttgtctccaggggaaagagccaccctctcctgcagggccagtcagagtgttatcagcatctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcagttttggccaggggaccaaactggagatcaaa

SEQ ID NO: 86 - protein, 108 aa, Artificial Sequence
  Description of Artificial Sequence Synthetic polypeptide construct
EIVLTQSPGTLSLSPGERATLSCRASQSVISIYLAWYQQKPGQAPRLLIYGASSRATGIPDRFSGSGSGTDFTLTISRLEPEDFAVYYCQQYGSSPCSFGQGTKLEIK

SEQ ID NO: 87 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaggagtcgagttaccatatcagtagacacgtctaagaaccagttctccctgaagctgagctctgtgactgccgcggacacggccgtgtattactgtgcgagagcggattacgatttttggagtggttattttgactactggggccagggaaccctggtcaccgtctcctca

SEQ ID NO: 88 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLRSRVTISVDTSKNQFSLKLSSVTAADTAVYYCARADYDFWSGYFDYWGQGTLVTVSS

SEQ ID NO: 89 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagatacctgggaaagcccctaagcgcctgatctatgctgcatccagtttgcaaagtggggtcccatcaaggttcagcggcagtggatctgggacagaattcactctcacaatcaacagcctgcagcctgaagattttgcaacttattactgtctacagcataatggttacccgtggacgttcggccaagggaccaaggtggaaatcaaac

SEQ ID NO: 90 - protein, 107 aa, Artificial Sequence
  Description of Artificial Sequence Synthetic polypeptide construct
DIQMTQSPSSLSASVGDRVTITCRASQGIRNDLGWYQQIPGKAPKRLIYAASSLQSGVPSRFSGSGSGTEFTLTINSLQPEDFATYYCLQHNGYPWTFGQGTKVEIK

SEQ ID NO: 91 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattggatacatctattacagtgggagcacctactacaacccgtccctcaagagtcgagttaccatatcagtagacacgtctaagaaccagttctccctgaagctgagctctgtgactgccgcggacacggccgtgtattactgtgcgagagcggattacgatttttggagtggttattttgactactggggccagggaaccctggtcaccgtctcctca

SEQ ID NO: 92 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWSGYFDYWGQGTLVTVSS

SEQ ID NO: 93 - DNA, 322 bp, Artificial Sequence
  Description of Artificial Sequence Synthetic polynucleotide construct
gacttccagatgacccagtctccatcctccctgtctgcatctgtaggagacagagtcaccatcacttgccgggcaagtcaggacattcgaaatgatttaggctggtatcggcagaaacctgggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 94 - protein, 107 aa, Artificial Sequence
  Description of Artificial Sequence Synthetic polypeptide construct
DFQMTQSPSSLSASVGDRVTITCRASQDIRNDLGWYRQKPGKAPKRLIYAASSLQSGVPSRFSGSGSGTEFTLTISSLQPEDFATYYCLQHNSYPWTFGQGTKVEIK

SEQ ID NO: 95 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattggatacatctattacagtgggagcacctactacaacccgtccctcaagagtcgagttaccatatcaatagacacgtctaagaaccagttctccctgaagctgagctctgtgactgccgcggacacggccgtgtattactgtgcgagagcggattacgatttttggaatggttattttgactactggggccagggaaccctggtcaccgtctcctca

SEQ ID NO: 96 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISIDTSKNQFSLKLSSVTAADTAVYYCARADYDFWNGYFDYWGQGTLVTVSS

SEQ ID NO: 97 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcaggacattagaaatgatttaggctggtatcggcagaaacctgggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaa

SEQ ID NO: 98 - protein, 107 aa, Artificial Sequence
  Description of Artificial Sequence Synthetic polypeptide construct
DIQMTQSPSSLSASVGDRVTITCRASQDIRNDLGWYRQKPGKAPKRLIYAASSLQSGVPSRFSGSGSGTEFTLTISSLQPEDFATYYCLQHNSYPWTFGQGTKVEIK

SEQ ID NO: 99 - DNA, 366 bp, Artificial Sequence
  Description of Artificial Sequence Synthetic polynucleotide construct
caggtgcagctgcaggagtcgggcccaggactggtgaagcctacacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgacttaccatatcagtagacacgtctaagaaccagttctccctgaagctgagctctgtgactgccgcggacacggccgtgtattactgtgcgagagcagattacgatttttggagtggttactttgactactggggccagggaaccctggtcaccgtctcctca

SEQ ID NO: 100 - protein, 122 aa, Artificial Sequence
  Description of Artificial Sequence Synthetic polypeptide construct
QVQLQESGPGLVKPTQTLSLTCTVSGGSISSGDYYWSWIRQHPGKGLEWIGYIYYSGSTYYNPSLKSRLTISVDTSKNQFSLKLSSVTAADTAVYYCARADYDFWSGYFDYWGQGTLVTVSS

SEQ ID NO: 101 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataataattacccgtggacgttcggccaagggaccaaggtggaaatcaaa

SEQ ID NO: 102 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPWTFGQGTKVEIK

SEQ ID NO: 103 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagtagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagttgagctctgtgactgccgcggacacggccgtgtattactgtgcgagagccgattacgatttttggagtggttattttgactactggggccagggaaccctggtcaccgtctcctca

SEQ ID NO: 104 - protein, 122 aa, Artificial Sequence; identical to SEQ ID NO: 92
  Description of Artificial Sequence Synthetic polypeptide construct

SEQ ID NO: 105 - DNA, 321 bp, Artificial Sequence; identical to SEQ ID NO: 101
  Description of Artificial Sequence Synthetic polynucleotide construct

SEQ ID NO: 106 - protein, 107 aa, Artificial Sequence; identical to SEQ ID NO: 102
  Description of Artificial Sequence Synthetic polypeptide construct

SEQ ID NO: 107 - DNA, 351 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagaggacgacggtatggacgtctggggccaagggaccacggtcaccgtctcctca

SEQ ID NO: 108 - protein, 117 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAREDDGMDVWGQGTTVTVSS

SEQ ID NO: 109 - DNA, 336 bp, Artificial Sequence
  Description of Artificial Sequence Synthetic polynucleotide construct
gatattgtgatgactcagtctccactctccctgcccgtcacccctggagagccggcctccatttcctgcaggtctagtcagagcctcctgcatagtaatggatacaactatttggaatggtacctgcagaagccagggcagtccccacagttcatgatttatttggggtctaatcgggcctccggggtccctgacaggttcagtggcagtggatcaggcacagattttacactgaaaatcagcagagtggaggctgaggatgttggggtttattactgcatgcaagctctacaaactccgatcaccttcggccaagggacacgactggagattaaa

SEQ ID NO: 110 - protein, 112 aa, Artificial Sequence
  Description of Artificial Sequence Synthetic polypeptide construct
DIVMTQSPLSLPVTPGEPASISCRSSQSLLHSNGYNYLEWYLQKPGQSPQFMIYLGSNRASGVPDRFSGSGSGTDFTLKISRVEAEDVGVYYCMQALQTPITFGQGTRLEIK

SEQ ID NO: 111 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagtacccagggaagggcctggagtggattgggtacatctattacagtgggagcacctactacaacccgtccctcaagagtcgagttaccatatcagtagacacgtctaagaaccagttctccctgaagctgaggtctgtgactgccgcggacacggccgtgtattactgtgcgagagcggattacgatttttggagtggttattttgactactggggccagggaaccctggtcaccgtctcctca

SEQ ID NO: 112 - protein, 122 aa, Artificial Sequence
  Description of Artificial Sequence Synthetic polypeptide construct
QVQLQESGPGLVKPSQTLSLTCTVSGGSISSGDYYWSWIRQYPGKGLEWIGYIYYSGSTYYNPSLKSRVTISVDTSKNQFSLKLRSVTAADTAVYYCARADYDFWSGYFDYWGQGTLVTVSS

SEQ ID NO: 113 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggaaatcaaac

SEQ ID NO: 114 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPWTFGQGTKVEIK

SEQ ID NO: 115 - DNA, 372 bp, Artificial Sequence
  Description of Artificial Sequence Synthetic polynucleotide construct
caggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagatggggacgtggatacagctatggtcgatgcttttgatatctggggccaagggacaatggtcaccgtctcctca

SEQ ID NO: 116 - protein, 124 aa, Artificial Sequence
  Description of Artificial Sequence Synthetic polypeptide construct
QVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCARDGDVDTAMVDAFDIWGQGTMVTVSS

SEQ ID NO: 117 - DNA, 324 bp, Artificial Sequence
  Description of Artificial Sequence Synthetic polynucleotide construct
gaaattgtattgacgcagtctccaggcaccctgtctttgtctccaggggaaagagccaccctctcctgcagggccagtcagagtttaagcggcaactacttagcctggtaccagcagaagcctggccaggctcccaggctcatcatctgtggtgcatccagcagggccactggcatcccagacaggttcagtggcagtgggtctgggacagacttcactctcaccatcacaagactggagcctgaagattttgcagtgtattactgtcagcagtatgataggtcaccgctcactttcggcggagggaccaaggtggagatcaaa

SEQ ID NO: 118 - protein, 108 aa, Artificial Sequence
  Description of Artificial Sequence Synthetic polypeptide construct
EIVLTQSPGTLSLSPGERATLSCRASQSLSGNYLAWYQQKPGQAPRLIICGASSRATGIPDRFSGSGSGTDFTLTITRLEPEDFAVYYCQQYDRSPLTFGGGTKVEIK

SEQ ID NO: 119 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaggagattacgatttttggagtggagagtttgactactggggccagggaaccctggtcaccgtctcctca

SEQ ID NO: 120 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARGDYDFWSGEFDYWGQGTLVTVSS

SEQ ID NO: 121 - DNA, 386 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctatgacagtgggagcacctactacaacccgtccctcaagagtcgagttaccatatcagtagacacgtctaagaaccagttctccctgaagctgaggtctgtgactgccgcggacacggccgtgtattactgtgcgagagatcaggggcaggacggatacagctatggttacggctactactacggtatggacgtctggggccaagggaccacggtcaccgtctcctc

SEQ ID NO: 122 - protein, 128 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYDSGSTYYNPSLKSRVTISVDTSKNQFSLKLRSVTAADTAVYYCARDQGQDGYSYGYGYYYGMDVWGQGTTVTVS

SEQ ID NO: 123 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccaggcgagtcaggacattagcaattatttaaattggtatcagcagaaaccagggaaagcccctaaactcctgatctacgttgcatccaatttggaaacaggggtcccatcaaggttcagtggaagtggatctgggacagattttactttcaccatcagcagcctgcagcctgaagatattgcaacatattactgtcaacagtgtgataatctccctctcactttcggcggagggaccaaggtggagatcaaa

SEQ ID NO: 124 - protein, 107 aa, Artificial Sequence
  Description of Artificial Sequence Synthetic polypeptide construct
DIQMTQSPSSLSASVGDRVTITCQASQDISNYLNWYQQKPGKAPKLLIYVASNLETGVPSRFSGSGSGTDFTFTISSLQPEDIATYYCQQCDNLPLTFGGGTKVEIK

SEQ ID NO: 125 - DNA, 365 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattggatacatctattacagtgggagcacctactacaacccgtccctcaagagtcgagttaccatatcagtagacacgtctaagaaccagttctccctgaagctgagctctgtgactgccgcggacacggccgtgtattactgtgcgagagcggattacgatttttggagtggttattttgactactggggccagggaaccctggtcaccgtctcctc

SEQ ID NO: 126 - protein, 121 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWSGYFDYWGQGTLVTVS

SEQ ID NO: 127 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcaggacattagaaatgatttaggctggtatcggcagaaacctgggaaagcccctaagcgcctgatctatgctgcatcccgtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 128 - protein, 107 aa, Artificial Sequence
  Description of Artificial Sequence Synthetic polypeptide construct
DIQMTQSPSSLSASVGDRVTITCRASQDIRNDLGWYRQKPGKAPKRLIYAASRLQSGVPSRFSGSGSGTEFTLTISSLQPEDFATYYCLQHNSYPWTFGQGTKVEIK

SEQ ID NO: 129 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagccgattacgatttttggagtggttattttgactactggggccagggaaccctggtcaccgtctcctca

SEQ ID NO: 130 - protein, 122 aa, Artificial Sequence; identical to SEQ ID NO: 92
  Description of Artificial Sequence Synthetic polypeptide construct

SEQ ID NO: 131 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaaatggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 132 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQNGVPSRFSGSGSGTEFTLTISSLQPEDFATYYCLQHNSYPWTFGQGTKVEIK

SEQ ID NO: 133 - DNA, 365 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagcggattacgatttttggagtggttattttgactactggggccagggaatcctggtcaccgtctcctc

SEQ ID NO: 134 - protein, 121 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWSGYFDYWGQGILVTVS

SEQ ID NO: 135 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatttatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 136 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 137 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagtagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagttgagctctgtgactgccgcggacacggccgtgtattactgtgcgagagccgattacgatttttggaatggttattttgactactggggccagggaaccctggtcaccgtctcctca

SEQ ID NO: 138 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWNGYFDYWGQGTLVTVSS

SEQ ID NO: 139 - DNA, 321 bp, Artificial Sequence; identical to SEQ ID NO: 101
  Description of Artificial Sequence Synthetic polynucleotide construct

SEQ ID NO: 140 - protein, 107 aa, Artificial Sequence; identical to SEQ ID NO: 102
  Description of Artificial Sequence Synthetic polypeptide construct

SEQ ID NO: 141 - DNA, 366 bp, Artificial Sequence; identical to SEQ ID NO: 129
  Description of Artificial Sequence Synthetic polynucleotide construct

SEQ ID NO: 142 - protein, 122 aa, Artificial Sequence; identical to SEQ ID NO: 92
  Description of Artificial Sequence Synthetic polypeptide construct

SEQ ID NO: 143 - DNA, 322 bp, Artificial Sequence
  Description of Artificial Sequence Synthetic polynucleotide construct
gacatccagctgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaaatggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 144 - protein, 107 aa, Artificial Sequence
  Description of Artificial Sequence Synthetic polypeptide construct
DIQLTQSPSSLSASVGDRVTITCRASQGIRNDLGWYQQKPGKAPKRLIYAASSLQNGVPSRFSGSGSGTEFTLTISSLQPEDFATYYCLQHNSYPWTFGQGTKVEIK

SEQ ID NO: 145 - DNA, 365 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggtgattactactggagctggatccgccagtacccagggaagggcctggagtggattgggtacatctattacagtgggagcacctactacaacccgtccctcaagagtcgagttaccatatcagtagacacgtctaagaaccagttctccctgaagctgggctctgtgactgccgcggacacggccgtgtatttctgtgcgagagccgattacgatttttggagtggttattttgacttctggggccagggaaccctggtcaccgtctcctc

SEQ ID NO: 146 - protein, 121 aa, Artificial Sequence
  Description of Artificial Sequence Synthetic polypeptide construct
QVQLQESGPGLVKPSQTLSLTCTVSGGSISSGDYYWSWIRQYPGKGLEWIGYIYYSGSTYYNPSLKSRVTISVDTSKNQFSLKLGSVTAADTAVYFCARADYDFWSGYFDFWGQGTLVTVS

SEQ ID NO: 147 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatggttacccgtggacgttcggccaagggaccaaggtggaaatcaaac

SEQ ID NO: 148 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQSGVPSRFSGSGSGTEFTLTISSLQPEDFATYYCLQHNGYPWTFGQGTKVEIK

SEQ ID NO: 149 - DNA, 365 bp, Artificial Sequence; identical to SEQ ID NO: 145
  Description of Artificial Sequence Synthetic polynucleotide construct

SEQ ID NO: 150 - protein, 121 aa, Artificial Sequence; identical to SEQ ID NO: 146
  Description of Artificial Sequence Synthetic polypeptide construct

SEQ ID NO: 151 - DNA, 322 bp, Artificial Sequence; identical to SEQ ID NO: 147
  Description of Artificial Sequence Synthetic polynucleotide construct

SEQ ID NO: 152 - protein, 107 aa, Artificial Sequence; identical to SEQ ID NO: 148
  Description of Artificial Sequence Synthetic polypeptide construct

SEQ ID NO: 153 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagtagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagcggattacgatttttggagtggttattttgactcctggggccagggaaccctggtcaccgtctcctca

SEQ ID NO: 154 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWSGYFDSWGQGTLVTVSS

SEQ ID NO: 155 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagatacctgggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatggttacccgtggacgttcggccaagggaccaaggtggaaatcaaa

SEQ ID NO: 156 - protein, 107 aa, Artificial Sequence
  Description of Artificial Sequence Synthetic polypeptide construct
DIQMTQSPSSLSASVGDRVTITCRASQGIRNDLGWYQQIPGKAPKRLIYAASSLQSGVPSRFSGSGSGTEFTLTISSLQPEDFATYYCLQHNGYPWTFGQGTKVEIK

SEQ ID NO: 157 - DNA, 366 bp, Artificial Sequence
  Description of Artificial Sequence Synthetic polynucleotide construct
caggttcagctggtgcagtctggagctgaggtgaagaagcctggggcctcagtgaaggtctcctgcaaggcttctggttacacctttaccaactatggtatcagctgggtgcggcaggcccctggacaagggcttgagtggatgggatggatcagcgcttacgatggttacagaaactatgcacagaagctccagggcagagtcaccatgaccacagacacatccacgaccactgcctacatggagctgaggagcctgagatctgacgacacggccgtgtattactgtgcgagagatgttcaagactacggtgactacgactactttgactactggggccagggaaccctggtcaccgtctcctca

SEQ ID NO: 158 - protein, 122 aa, Artificial Sequence
  Description of Artificial Sequence Synthetic polypeptide construct
QVQLVQSGAEVKKPGASVKVSCKASGYTFTNYGISWVRQAPGQGLEWMGWISAYDGYRNYAQKLQGRVTMTTDTSTTTAYMELRSLRSDDTAVYYCARDVQDYGDYDYFDYWGQGTLVTVSS

SEQ ID NO: 159 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagagcattagcagttatttaaattggtatcagcagaaaccagggaaagcccctaacctcctgatctatgctgcatccagtttgcaaagtggggtcccatcaagattcaggggcagtggatctgggacagatttcactctcaccatcagcagtctgcaacctgaagattttgcaacttactactgtcaacagagttacagtacccccatcaccttcggccaagggacacgactggagattaaa

SEQ ID NO: 160 - protein, 107 aa, Artificial Sequence
  Description of Artificial Sequence Synthetic polypeptide construct
DIQMTQSPSSLSASVGDRVTITCRASQSISSYLNWYQQKPGKAPNLLIYAASSLQSGVPSRFRGSGSGTDFTLTISSLQPEDFATYYCQQSYSTPITFGQGTRLEIK

SEQ ID NO: 161 - DNA, 366 bp, Artificial Sequence
  Description of Artificial Sequence Synthetic polynucleotide construct
caggtgcagctgcaggagtcgggcccaggactggtgaagcctttacagaccctgtccctcacctgcactgtctctggtggctccatcagcagtggtgattactactggagctggatccgccagcacccagggaagggcctggagtggattgggtacatctattacagtgggaccacctactacaacccgtccctcaagagtcgagttaccatatcagtagacacgtctaagaaccagttcgccctgaagctgaactctgtgactgccgcggacacggccgtgtattactgtgcgagagccgattacgatttttggagtggttattttgactactggggccagggaaccctggtcaccgtctcctca

SEQ ID NO: 162 - protein, 122 aa, Artificial Sequence
  Description of Artificial Sequence Synthetic polypeptide construct
QVQLQESGPGLVKPLQTLSLTCTVSGGSISSGDYYWSWIRQHPGKGLEWIGYIYYSGTTYYNPSLKSRVTISVDTSKNQFALKLNSVTAADTAVYYCARADYDFWSGYFDYWGQGTLVTVSS

SEQ ID NO: 163 - DNA, 322 bp, Artificial Sequence
  Description of Artificial Sequence Synthetic polynucleotide construct
gacatccagatgacccagtctccatcctccctgtctgcatctgtaggagacagagtcaccatcacttgccgggcaggtcagggcattagaaatgatttaggctggtatcagcagaaaccagggaaagcccctcagcgcctgatctatgctgcatccagtttgcaaagtggggtcccatcaaggttcagcggcagtggatctgggacagaattctctctcacaatctccagcctgcagcctgaagattttgcaacttattactgtctacagcataatagttacccgtggacgttcggccaagggaccaaggtggaaatcaaac

SEQ ID NO: 164 - protein, 107 aa, Artificial Sequence
  Description of Artificial Sequence Synthetic polypeptide construct
DIQMTQSPSSLSASVGDRVTITCRAGQGIRNDLGWYQQKPGKAPQRLIYAASSLQSGVPSRFSGSGSGTEFSLTISSLQPEDFATYYCLQHNSYPWTFGQGTKVEIK

SEQ ID NO: 165 - DNA, 369 bp, Artificial Sequence
  Description of Artificial Sequence Synthetic polynucleotide construct
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatatattagtagtagtggtaataacatataccacgcagactctgtgaagggccgattcaccatctccagggacaacgccaagaactcactgtatctgcaaatgaacagcctgagagccgaggacacggccgtgtattactgtgcgagagagagatatagtggctacgacgaccctgatggttttgatatctggggccaagggacaatggtcaccgtctcttca

SEQ ID NO: 166 - protein, 123 aa, Artificial Sequence
  Description of Artificial Sequence Synthetic polypeptide construct
QVQLVESGGGLVKPGGSLRLSCAASGFTFSDYYMSWIRQAPGKGLEWVSYISSSGNNIYHADSVKGRFTISRDNAKNSLYLQMNSLRAEDTAVYYCARERYSGYDDPDGFDIWGQGTMVTVSS

SEQ ID NO: 167 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccaggcgagtcaggacattagcaactatttaagttggtttcagcagaaaccagggaaagcccctaagctcctgatccacgatgcatccaatttggaaacaggggtcccttcaaggttcagtggaagtggatctgggacagattttactttcaccatcagcagcctgcagcctgaagatattgcaacatattactgtcaacagtatgataatcccccgtgcagttttggccaggggaccaagctggagatcaaa

SEQ ID NO: 168 - protein, 107 aa, Artificial Sequence
  Description of Artificial Sequence Synthetic polypeptide construct
DIQMTQSPSSLSASVGDRVTITCQASQDISNYLSWFQQKPGKAPKLLIHDASNLETGVPSRFSGSGSGTDFTFTISSLQPEDIATYYCQQYDNPPCSFGQGTKLEIK

SEQ ID NO: 169 - DNA, 366 bp, Artificial Sequence
  Description of Artificial Sequence Synthetic polynucleotide construct
caggtgcagctgcaggagtcgggcccaggactggtgaagccttcacagaccctgtccctcacctgcactgtctctggtggctccatcagcagtggttattactactggagctggatccgccagcacccagggaagggcctggagtggattgggtacatctattacagtgggaccacctactacaatccgtccttcaagagtcgagttaccatatcagtagacacgtctaagaaccagttctccctgaaactgagctctgtgactgccgcggacacggccgtgtattactgtgcgagagccgattacgatttttggagtggtcactttgactactggggccagggaaccctggtcaccgtctcctca

SEQ ID NO: 170 - protein, 122 aa, Artificial Sequence
  Description of Artificial Sequence Synthetic polypeptide construct
QVQLQESGPGLVKPSQTLSLTCTVSGGSISSGYYYWSWIRQHPGKGLEWIGYIYYSGTTYYNPSFKSRVTISVDTSKNQFSLKLSSVTAADTAVYYCARADYDFWSGHFDYWGQGTLVTVSS

SEQ ID NO: 171 - DNA, 321 bp, Artificial Sequence
  Description of Artificial Sequence Synthetic polynucleotide construct
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaa

SEQ ID NO: 172 - protein, 107 aa, Artificial Sequence; identical to SEQ ID NO: 136
  Description of Artificial Sequence Synthetic polypeptide construct

SEQ ID NO: 173 - DNA, 365 bp, Artificial Sequence
  Description of Artificial Sequence Synthetic polynucleotide construct
caggttcagctggtgcagtctggagctgaggtgaagaagcctggggcctcagtgaaggtctcctgcaaggcttctggttacacctttaccagctatggtatcagctgggtgcgacaggcccctggacaaggacttgagtggatgggatggatcagcgcttacgatggtcacacaaactatgcacagaagctccagggcagagtcaccatgaccacagacacatccacgaacacagcctacatggagctgaggagcctgagatctgacgacacggccgtttattactgtgcgagagacccccatgactacagtaactacgaggcttttgacttctggggccagggaaccctggtcaccgtctcctc

SEQ ID NO: 174 - protein, 121 aa, Artificial Sequence
  Description of Artificial Sequence Synthetic polypeptide construct
QVQLVQSGAEVKKPGASVKVSCKASGYTFTSYGISWVRQAPGQGLEWMGWISAYDGHTNYAQKLQGRVTMTTDTSTNTAYMELRSLRSDDTAVYYCARDPHDYSNYEAFDFWGQGTLVTVS

SEQ ID NO: 175 - DNA, 519 bp, Artificial Sequence
  Description of Artificial Sequence Synthetic polynucleotide construct
atgaggtcccctgctcagctcctggggctcctgctactctggctccgaggtgccagatgtgacatccagatgacccagtctccatcctccctgtctgcatctgtaggagacagagtcaccatcacttgccgggcaagtcagagcattagcagttatttaaattggtatcagcagaaaccagggaaagcccctaacctcctgatctatgctgcatccagtttgcaaagtggggtcccatcaagattcagtggcagtggatctgggacagatttcactctcaccatcagcagtctgcaacctgaagattttgcaacttactactgtcaacagagttacagtacccccatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 176 - protein, 107 aa, Artificial Sequence
  Description of Artificial Sequence Synthetic polypeptide construct
DIQMTQSPSSLSASVGDRVTITCRASQSISSYLNWYQQKPGKAPNLLIYAASSLQSGVPSRFSGSGSGTDFTLTISSLQPEDFATYYCQQSYSTPITFGQGTRLEIK

SEQ ID NO: 177 - DNA, 534 bp, Artificial Sequence
  Description of Artificial Sequence Synthetic polynucleotide construct
accatggactggacctggagggtccttttcttggtggcagcagcaacaggtgcccactcccaggttcagctggtgcagtctggagctgaggtgaagaagcctggggcctcagtgaaggtctcctgcaaggcttctggttacacctttaccaactatggtatcagctgggtgcggcaggcccctggacaagggcttgagtggatgggatggatcagcgcttacgatggttacagaaactatgcacagaagctccagggcagagtcaccatgaccacagacacatccacgaccactgcctacatggagctgaggagcctgagatctgacgacacggccgtgtattactgtgcgagagatgttcaagactacggtgactacgactactttgactactggggccagggaaccctggtcaccgtctcctcagcttccaccaagggcccatccgtcttccccctggtgccctgctccaggagcacctccgagagcacagccgccctgggctgcctggtcaaggactacttccccgaaccg

SEQ ID NO: 178 - protein, 122 aa, Artificial Sequence; identical to SEQ ID NO: 158
  Description of Artificial Sequence Synthetic polypeptide construct

SEQ ID NO: 179 - DNA, 504 bp, Artificial Sequence
  Description of Artificial Sequence Synthetic polynucleotide construct
cagctcctggggctcctgctactctggctccgaggtgccagatgtgacatccagatgacccagtctccatcctccctgtctgcatctgtaggagacagagtcaccatcacttgccgggcaagtcagagcattagcagttatttaaattggtatcagcagaaaccagggaaagcccctaacctcctgatctatgctgcatccagtttgcaaagtggggtcccatcaagattcagtggcagtggatctgggacagatttcactctcaccatcagcagtctgcaacctgaagattttgcaacttactactgtcaacagagttacagtacccccatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 180 - protein, 107 aa, Artificial Sequence; identical to SEQ ID NO: 176
  Description of Artificial Sequence Synthetic polypeptide construct

SEQ ID NO: 181 - DNA, 493 bp, Artificial Sequence
  Description of Artificial Sequence Synthetic polynucleotide construct
catctgtggttcttcctcctgctggtggcagctcccagatgggtcctgtcccaggtgcagctgcaggagtcgggcccaggactggtgaagccttcacagaccctgtccctcacctgcactgtctctggtggctccatcaacagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagcagattacgatttttggagtggttactttgactactggggccagggaaccctggtcaccgtctcctcagcctccaccaagggcccatcggtcttccccctggcaccctcctccaagagcacctctgggggcacaacggccctgg

SEQ ID NO: 182 - protein, 122 aa, Artificial Sequence
  Description of Artificial Sequence Synthetic polypeptide construct
QVQLQESGPGLVKPSQTLSLTCTVSGGSINSGDYYWSWIRQHPGKGLEWIGYIYYSGSTYYNPSLKSRVTISVDTSKNQFSLKLSSVTAADTAVYYCARADYDFWSGYFDYWGQGTLVTVSS

SEQ ID NO: 183 - DNA, 518 bp, Artificial Sequence
  Description of Artificial Sequence Synthetic polynucleotide construct
atgagggtccctgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 184 - protein, 107 aa, Artificial Sequence; identical to SEQ ID NO: 136
  Description of Artificial Sequence Synthetic polypeptide construct

SEQ ID NO: 185 - DNA, 436 bp, Artificial Sequence
  Description of Artificial Sequence Synthetic polynucleotide construct
tggttcttccttctgctggtggcagctcccagatgggtcctgtcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagatggctatgatagtagtggttattaccacggctactttgactactggggccagggaaccctggtcaccgtctcctcagcctccaccaagggcc

SEQ ID NO: 186 - protein, 125 aa, Artificial Sequence
  Description of Artificial Sequence Synthetic polypeptide construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDGYDSSGYYHGYFDYWGQGTLVTVSS

SEQ ID NO: 187 - DNA, 521 bp, Artificial Sequence
  Description of Artificial Sequence Synthetic polynucleotide construct
caggtcttcatttctctgttgctctggatctctggtgcctacggggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 188 - protein, 113 aa, Artificial Sequence
  Description of Artificial Sequence Synthetic polypeptide construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 189 - DNA, 455 bp, Artificial Sequence
  Description of Artificial Sequence Synthetic polynucleotide construct
ctgtggttcttcctcctgctggtggcagctcccagatgggtcctgtcccaggtgcagctgcaggagtcgggcccaggactggtgaagccttcacagaccctgtccctcacctgcactgtctctggtggctccatcagtagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagttgagctctgtgactgccgcggacacggccgtgtattactgtgcgagagccgattacgatttttggagtggttattttgactactggggccagggaaccctggtcaccgtctcctcagcctccaccaagggcccatcggtcttccccctggcaccctc

SEQ ID NO: 190 - protein, 122 aa, Artificial Sequence; identical to SEQ ID NO: 92
  Description of Artificial Sequence Synthetic polypeptide construct

SEQ ID NO: 191 - DNA, 442 bp, Artificial Sequence
  Description of Artificial Sequence Synthetic polynucleotide construct
gtgcccgctcagcgcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataataattacccgtggacgttcggccaagggaccaaggtggaaatcaaacgaactgtggctgcaccatctgtcttcatcttcccgccatctgatgagcagttgaaatctggaactg

SEQ ID NO: 192 - protein, 107 aa, Artificial Sequence; identical to SEQ ID NO: 102
  Description of Artificial Sequence Synthetic polypeptide construct

SEQ ID NO: 193 - DNA, 427 bp, Artificial Sequence
  Description of Artificial Sequence Synthetic polynucleotide construct
tggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtatttctgtgcgagagccgattacgatttttggagtggttattttgactactggggccagggaaccctggtcaccgtctcctcagcctccaccaagggcc

SEQ ID NO: 194 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYFCARADYDFWSGYFDYWGQGTLVTVSS

SEQ ID NO: 195 - DNA, 518 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 196 - protein, 107 aa, Artificial Sequence; identical to SEQ ID NO: 114
  Description of Artificial Sequence Synthetic polypeptide construct

SEQ ID NO: 197 - DNA, 428 bp, Artificial Sequence
  Description of Artificial Sequence Synthetic polynucleotide construct
tggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtatttctgtgcgagagccgattacgatttttggaatggttattttgactactggggccagggaaccctggtcaccgtctcctcagcctccaccaagggccc

SEQ ID NO: 198 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYFCARADYDFWNGYFDYWGQGTLVTVSS

SEQ ID NO: 199 - DNA, 519 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcttccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 200 - protein, 107 aa, Artificial Sequence; identical to SEQ ID NO: 114
  Description of Artificial Sequence Synthetic polypeptide construct

SEQ ID NO: 201 - DNA, 398 bp, Artificial Sequence
  Description of Artificial Sequence Synthetic polynucleotide construct
ttggtggcagcagctacaggcacccacgcccaggtccagctggtacagtctggggctgaggtgaagaagcctggggcctcagtgaaggtctcctgcaaggtttccggatacaccctcactgaattatccatgtactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaactgggtggaactacgtctttgactactggggccagggaaccctggtcaccgtctcctcagcctccaccaagggccc

SEQ ID NO: 202 - protein, 117 aa, Artificial Sequence
  Description of Artificial Sequence Synthetic polypeptide construct
QVQLVQSGAEVKKPGASVKVSCKVSGYTLTELSMYWVRQAPGKGLEWMGGFDPEDGETIYAQKFQGRVTMTEDTSTDTAYMELSSLRSEDTAVYYCATGWNYVFDYWGQGTLVTVSS

SEQ ID NO: 203 - DNA, 388 bp, Artificial Sequence
  Description of Artificial Sequence Synthetic polynucleotide construct
ggatccagtggggatattgtgatgactcagtctccactctccctgcccgtcacccctggagagccggcctccatctcctgcaggtccagtcagagcctcctgcatagtaatggatacaactatttggattggtacctgcagaagccagggcagtctccacagctcctgatctatttggattctcatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaacgaactgtggctgcaccatctgtcttcatcttcccgccat

SEQ ID NO: 204 - protein, 112 aa, Artificial Sequence
  Description of Artificial Sequence Synthetic polypeptide construct
DIVMTQSPLSLPVTPGEPASISCRSSQSLLHSNGYNYLDWYLQKPGQSPQLLIYLDSHRASGVPDRFSGSGSGTDFTLKISRVEAEDVGVYYCMQALQTPLTFGGGTKVEIK

SEQ ID NO: 205 - DNA, 446 bp, Artificial Sequence
  Description of Artificial Sequence Synthetic polynucleotide construct
tggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtatttctgtgcgagagccgattacgatttttggagtggttattttgactactggggccagggaaccctggtcaccgtctcctcagcctccaccaagggcccatcgagtcttccccctgg

SEQ ID NO: 206 - protein, 122 aa, Artificial Sequence; identical to SEQ ID NO: 194
  Description of Artificial Sequence Synthetic polypeptide construct

SEQ ID NO: 207 - DNA, 519 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacaaaattcactctcactatcagcagcctgcagcctgaagattttgcaacttattactgtctacagcataatacttacccgtggacgttcggccaagggaccaaggtggaaatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 208 - protein, 107 aa, Artificial Sequence
  Description of Artificial Sequence Synthetic polypeptide construct
DIQMTQSPSSLSASVGDRVTITCRASQGIRNDLGWYQQKPGKAPKRLIYAASSLQSGVPSRFSGSGSGTKFTLTISSLQPEDFATYYCLQHNTYPWTFGQGTKVEIR

SEQ ID NO: 209 - DNA, 564 bp, Artificial Sequence
  Description of Artificial Sequence Synthetic polynucleotide construct
accatgaaacatctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagcggattacgatttttggagtggttattttgactactggggccagggaatcctggtcaccgtctcctcagcctccaccaagggcccatcggtcttccccctggcaccctcctccaagaacacctctgggggcacagcggccctgggctgcctggtcaaggactacttccccgaaccggtgacggtgtcctggaactcaggcgccctg

SEQ ID NO: 210 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTISVDTSKNQFSLKLSSVTAADTAVYYCARADYDFWSGYFDYWGQGILVTVSS

SEQ ID NO: 211 - DNA, 519 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 212 - protein, 107 aa, Artificial Sequence; identical to SEQ ID NO: 136
  Description of Artificial Sequence Synthetic polypeptide construct

SEQ ID NO: 213 - DNA, 432 bp, Artificial Sequence
  Description of Artificial Sequence Synthetic polynucleotide construct
tggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattggatacatctattacagtgggagcacctactacaattcgtccctcaagagtcgagttaccatatcagtagacacgtctaagaaccagttctccctgaagctgagctctgtgactgccgcggacacggccgtgtattactgtgcgagagcggattacgatttttggagtggttattttgactactggggccagggaaccctggtcaccgtctcctcagcctccaccaagggcccatcg

SEQ ID NO: 214 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNSSLKSRVTISVDTSKNQFSLKLSSVTAADTAVYYCARADYDFWSGYFDYWGQGTLVTVSS

SEQ ID NO: 215 - DNA, 372 bp, Artificial Sequence
  Description of Artificial Sequence Synthetic polynucleotide construct
ggtgccaggtgtgacatccagatgacccagtctccatcctccctgtctgcatctgtaggagacagagtcaccatcacttgccgggcaagtcagggcattagaaatgatttaggctggtatcagcagaaacctgggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcacaatagttacccgtggacgttcggccaagggaccaaggtggaaatcaaacgaactgtggctgcaccatctgtcttcatcttcccgcca

SEQ ID NO: 216 - protein, 107 aa, Artificial Sequence; identical to SEQ ID NO: 136
  Description of Artificial Sequence Synthetic polypeptide construct

SEQ ID NO: 217 - DNA, 548 bp, Artificial Sequence
  Description of Artificial Sequence Synthetic polynucleotide construct
aggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattggatacatctattacagtgggagcacctactacaacccgtccctcaagagtcgagttaccatatcagtagacacgtctaagaaccagttctccctgaagctgagctctgtgactgccgcggacacggccgtgtattactgtgcgagagccgattacgatttttggagtggttattttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccct

SEQ ID NO: 218 - protein, 122 aa, Artificial Sequence; identical to SEQ ID NO: 92
  Description of Artificial Sequence Synthetic polypeptide construct

SEQ ID NO: 219 - DNA, 517 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacg

SEQ ID NO: 220 - protein, 107 aa, Artificial Sequence; identical to SEQ ID NO: 136
  Description of Artificial Sequence Synthetic polypeptide construct

SEQ ID NO: 221 - DNA, 446 bp, Artificial Sequence
  Description of Artificial Sequence Synthetic polynucleotide construct
ctgtggttcttccttctgctggtggcagctcccagatgggtcctgtcccaggtgcagctgcaggagtcgggcccaggactggtgaagccttcacagaccctgtccctcacctgcactgtctctggtggctccatcagcagtggtgattactactggagctggatccgccagcacccagggaagggcctggagtggattgggtacatctattacagtgggagcacctactacaacccgtccctcaagagtcgagttaccatgtcagtagacacgtctaagaaccagttctccctgaagctgagctctgtgactgccgcggacacggccgtgtattactgtgcgagagccgattacgatttttggagtggtcactttgactgctggggccagggaaccctggtcaccgtctcctcagcttccaccaagggccccatccgtcttccccc

SEQ ID NO: 222 - protein, 122 aa, Artificial Sequence
  Description of Artificial Sequence Synthetic polypeptide construct
QVQLQESGPGLVKPSQTLSLTCTVSGGSISSGDYYWSWIRQHPGKGLEWIGYIYYSGSTYYNPSLKSRVTMSVDTSKNQFSLKLSSVTAADTAVYYCARADYDFWSGHFDCWGQGTLVTVSS

SEQ ID NO: 223 - DNA, 419 bp, Artificial Sequence
  Description of Artificial Sequence Synthetic polynucleotide construct
atgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagagatgatttaggctggtatcagcagaaaccagggaaagcccctaagcgcctgatctatgctgaatccagtttgcaaagtggggtcccatcaaggttcagcggcagtggatctgggacagaattcactctcacaatcagcagcctgcagcctgaagattttgcaacttattactgtctacagcatcatagttacccgtggacgttcggccaagggaccaaggtggaaatcaaacgaactgtggctgcaccatctgtcttcatcttcccgcc

SEQ ID NO: 224 - protein, 107 aa, Artificial Sequence
  Description of Artificial Sequence Synthetic polypeptide construct
DIQMTQSPSSLSASVGDRVTITCRASQGIRDDLGWYQQKPGKAPKRLIYAESSLQSGVPSRFSGSGSGTEFTLTISSLQPEDFATYYCLQHHSYPWTFGQGTKVEIK

SEQ ID NO: 225 - DNA, 504 bp, Artificial Sequence
  Description of Artificial Sequence Synthetic polynucleotide construct
tggctgagctgggttttcctcgttgctcttttaagaggtgtccagtgtcaggtgcagctggtggagtctgggggaggcgtggtccagcctgggaggtccctgagactctcctgtgcagcgtctggattcaccttcaatagctatgacatgcactgggtccgccaggctccaggcaaggggctggagtgggtggcagttatatggtatgatggaagtaataaatactatgcagactccgtgaagggccgattcaccatctctagagacaattccaagaacacgctgtatctgcaaatgaacagcctgagagccgaggacacggctgtgtattactgtgcgagagaccgcttgtgtactaatggtgtatgctatgaagactacggtatggacgtctggggccaagggaccacggtcaccgtctcctcagcttccaccaagggcccatccgtcttccccctggcgccctgctccaggagcacctccgagagcacagccgccctgggc

SEQ ID NO: 226 - protein, 126 aa, Artificial Sequence
  Description of Artificial Sequence Synthetic polypeptide construct
QVQLVESGGGVVQPGRSLRLSCAASGFTFNSYDMHWVRQAPGKGLEWVAVIWYDGSNKYYADSVKGRFTISRDNSKNTLYLQMNSLRAEDTAVYYCARDRLCTNGVCYEDYGMDVWGQGTTVTVSS

SEQ ID NO: 227 - DNA, 504 bp, Artificial Sequence
  Description of Artificial Sequence Synthetic polynucleotide construct
atgagggtccctgctcagctcctggggctcctgctgctctggctctcaggtgccagatgtgacatccagatgacccagtctccatcctccctgtctgcatctgtaggagacagagtcaccatcacttgccaggcgagtcaggacattagcaactatttaaattggtatcagcagaaaccagggaaagcccctaaggtcctgatctacgatgcatccaatttggaaacaggggtcccatcaaggttcagtggaagtggatctgggacagattttactttcaccatcagcagcctgcagcctgaagatgttgcaacatattactgtcaacactatgatactctcccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtgg

SEQ ID NO: 228 - protein, 107 aa, Artificial Sequence
  Description of Artificial Sequence Synthetic polypeptide construct
DIQMTQSPSSLSASVGDRVTITCQASQDISNYLNWYQQKPGKAPKVLIYDASNLETGVPSRFSGSGSGTDFTFTISSLQPEDVATYYCQHYDTLPLTFGGGTKVEIK

SEQ ID NO: 229 - DNA, 472 bp, Artificial Sequence
  Description of Artificial Sequence Synthetic polynucleotide construct
ggactgtgcaagaacatgaaacacctgtggttcttcctcctgctggtggcagctcccagatgggtcctgtcccaggtgcagctgcaggagtcgggcccaggactggtgaagcctttacagaccctgtccctcacctgcactgtctctggtggctccatcagcagtggtgattactactggagctggatccgccagcacccagggaagggcctggagtggattgggtacatctattacagtgggaccacctactacaacccgtccctcaagagtcgagttaccatatcagtagacacgtctaagaaccagttcgccctgaagctgaactctgtgactgccgcggacacggccgtgtattactgtgcgagagccgattacgatttttggagtggttattttgactactggggccagggaaccctggtcaccgtctcctcagcttccaccaagggcccatccgtcttccccctgg

SEQ ID NO: 230 - protein, 122 aa, Artificial Sequence; identical to SEQ ID NO: 162
  Description of Artificial Sequence Synthetic polypeptide construct

SEQ ID NO: 231 - DNA, 531 bp, Artificial Sequence
  Description of Artificial Sequence Synthetic polynucleotide construct
atgagggtccctgctcagctcctggggctcctgctgctctggttcccaggtgccaggtgtgacatccagatgacccagtctccatcctccctgtctgcatctgtaggagacagagtcaccatcacttgccgggcaggtcagggcattagaaatgatttaggctggtatcagcagaaaccagggaaagcccctcagcgcctgatctatgctgcatccagtttgcaaagtggggtcccatcaaggttcagcggcagtggatctgggacagaattctctctcacaatctccagcctgcagcctgaagattttgcaacttattactgtctacagcataatagttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcccttccaatcggg

SEQ ID NO: 232 - protein, 107 aa, Artificial Sequence; identical to SEQ ID NO: 164
  Description of Artificial Sequence Synthetic polypeptide construct

SEQ ID NO: 233 - DNA, 26 bp, Artificial Sequence
  Description of Artificial Sequence Synthetic primer
cgggatccatgtcctagcctaggggc

SEQ ID NO: 234 - DNA, 39 bp, Artificial Sequence
  Description of Artificial Sequence Synthetic primer
gctctagattaatgatgatgatgatgatgttgtcctaaa

SEQ ID NO: 235 - protein, 12 aa, Artificial Sequence
  Description of Artificial Sequence Synthetic peptide
GGSINSGDYYWS

SEQ ID NO: 236 - protein, 12 aa, Artificial Sequence
  Description of Artificial Sequence Synthetic peptide
GGSISSGDYYWS

SEQ ID NO: 237 - protein, 12 aa, Artificial Sequence
  Description of Artificial Sequence Synthetic peptide
GGSISSGGYYWS

SEQ ID NO: 238 - protein, 10 aa, Artificial Sequence
  Description of Artificial Sequence Synthetic peptide
GYTLTELSMY

SEQ ID NO: 239 - protein, 12 aa, Artificial Sequence
  Description of Artificial Sequence Synthetic peptide
GGSVSSGGYYWS

SEQ ID NO: 240 - protein, 10 aa, Artificial Sequence
  Description of Artificial Sequence Synthetic peptide
GFTFNSYDMH

SEQ ID NO: 241 - protein, 10 aa, Artificial Sequence
  Description of Artificial Sequence Synthetic peptide
GYTFTNYGIS

SEQ ID NO: 242 - protein, 10 aa, Artificial Sequence
  Description of Artificial Sequence Synthetic peptide
GFTFSDYYMS

SEQ ID NO: 243 - protein, 12 aa, Artificial Sequence
  Description of Artificial Sequence Synthetic peptide
GGSISSGYYYWS

SEQ ID NO: 244 - protein, 10 aa, Artificial Sequence
  Description of Artificial Sequence Synthetic peptide
GYTFTSYGIS

SEQ ID NO: 245 - protein, 12 aa, Artificial Sequence
  Description of Artificial Sequence Synthetic peptide
GFSLSTSGVGVG

SEQ ID NO: 246 - protein, 10 aa, Artificial Sequence
  Description of Artificial Sequence Synthetic peptide
GFTVSSNYMS

SEQ ID NO: 247 - protein, 10 aa, Artificial Sequence
  Description of Artificial Sequence Synthetic peptide
GYSFTSYWIG

SEQ ID NO: 248 - protein, 10 aa, Artificial Sequence
  Description of Artificial Sequence Synthetic peptide
GYTFTSYDIN

SEQ ID NO: 249 - protein, 12 aa, Artificial Sequence
  Description of Artificial Sequence Synthetic peptide
GDSVSSNSAAWN

SEQ ID NO: 250 - protein, 10 aa, Artificial Sequence
  Description of Artificial Sequence Synthetic peptide
GGSISSYYWS

SEQ ID NO: 251 - protein, 10 aa, Artificial Sequence
  Description of Artificial Sequence Synthetic peptide
GYTFTGYYMH

SEQ ID NO: 252 - protein, 10 aa, Artificial Sequence
  Description of Artificial Sequence Synthetic peptide
GFTFSIYSMN

SEQ ID NO: 253 - protein, 12 aa, Artificial Sequence
  Description of Artificial Sequence Synthetic peptide
GGSVSSGGYYWN

SEQ ID NO: 254 - protein, 12 aa, Artificial Sequence
  Description of Artificial Sequence Synthetic peptide
GGSVSSGGYYWN

SEQ ID NO: 255 - protein, 10 aa, Artificial Sequence
  Description of Artificial Sequence Synthetic peptide
GFTFSSYGMH

SEQ ID NO: 256 - protein, 10 aa, Artificial Sequence
  Description of Artificial Sequence Synthetic peptide
GGSFSGYYWS

SEQ ID NO: 257 - protein, 12 aa, Artificial Sequence
  Description of Artificial Sequence Synthetic peptide
GVSISSGGYYWS

SEQ ID NO: 258 - protein, 16 aa, Artificial Sequence
  Description of Artificial Sequence Synthetic peptide
YIYYSGSTYYNPSLKS

SEQ ID NO: 259 - protein, 16 aa, Artificial Sequence
  Description of Artificial Sequence Synthetic peptide
YIYYSGSTYYNPSLRS

SEQ ID NO: 260 - protein, 17 aa, Artificial Sequence
  Description of Artificial Sequence Synthetic peptide
GFDPEDGETIYAQKFQG

SEQ ID NO: 261 - protein, 16 aa, Artificial Sequence
  Description of Artificial Sequence Synthetic peptide
YIYYSGSTNYNPSLKS

SEQ ID NO: 262 - protein, 16 aa, Artificial Sequence
  Description of Artificial Sequence Synthetic peptide
YIYYSGSTYYNSSLKS

SEQ ID NO: 263 - protein, 16 aa, Artificial Sequence
  Description of Artificial Sequence Synthetic peptide
YIYDSGSTYYNPSLKS

SEQ ID NO: 264 - protein, 17 aa, Artificial Sequence
  Description of Artificial Sequence Synthetic peptide
VIWYDGSNKYYADSVKG

SEQ ID NO: 265 - protein, 16 aa, Artificial Sequence
  Description of Artificial Sequence Synthetic peptide
YIYYSGTTYYNPSLKS

SEQ ID NO: 266 - protein, 17 aa, Artificial Sequence
  Description of Artificial Sequence Synthetic peptide
WISAYDGYRNYAQKLQG

SEQ ID NO: 267 - protein, 17 aa, Artificial Sequence
  Description of Artificial Sequence Synthetic peptide
YISSSGNNIYHADSVKG

SEQ ID NO: 268 - protein, 16 aa, Artificial Sequence
  Description of Artificial Sequence Synthetic peptide
YIYYSGTTYYNPSFKS

SEQ ID NO: 269 - protein, 17 aa, Artificial Sequence
  Description of Artificial Sequence Synthetic peptide
WISAYDGHTNYAQKLQG

SEQ ID NO: 270 - protein, 16 aa, Artificial Sequence
  Description of Artificial Sequence Synthetic peptide
LIYWNDDKRYSPSLKS

SEQ ID NO: 271 - protein, 16 aa, Artificial Sequence
  Description of Artificial Sequence Synthetic peptide
VIYSGGSTYYADSVKG

SEQ ID NO: 272 - protein, 16 aa, Artificial Sequence
  Description of Artificial Sequence Synthetic peptide
YIYSSGSTYYNPSLKS

SEQ ID NO: 273 - protein, 17 aa, Artificial Sequence
  Description of Artificial Sequence Synthetic peptide
IIYPGDSDTRYSPSFQG

SEQ ID NO: 274 - protein, 17 aa, Artificial Sequence
  Description of Artificial Sequence Synthetic peptide
IIWPGDSDTIYSPSFQG

SEQ ID NO: 275 - protein, 17 aa, Artificial Sequence
  Description of Artificial Sequence Synthetic peptide
WMNPNSGDTGYAQVFQG

SEQ ID NO: 276 - protein, 18 aa, Artificial Sequence
  Description of Artificial Sequence Synthetic peptide
RTYYRSKWYNDYAVSVKS

SEQ ID NO: 277 - protein, 16 aa, Artificial Sequence
  Description of Artificial Sequence Synthetic peptide
HIYTSGSTNYNPSLKS

SEQ ID NO: 278 - protein, 17 aa, Artificial Sequence
  Description of Artificial Sequence Synthetic peptide
WINPNIGGTNCAQKFQG

SEQ ID NO: 279 - protein, 16 aa, Artificial Sequence
  Description of Artificial Sequence Synthetic peptide
NIYYSGSTYYNPSLKS

SEQ ID NO: 280 - protein, 17 aa, Artificial Sequence
  Description of Artificial Sequence Synthetic peptide
YISSSSSTIYYADSVKG

SEQ ID NO: 281 - protein, 16 aa, Artificial Sequence
  Description of Artificial Sequence Synthetic peptide
YINYSGSTNYNPSLKS

SEQ ID NO: 282 - protein, 16 aa, Artificial Sequence
  Description of Artificial Sequence Synthetic peptide
EINHSGSTNYNPSLKS

SEQ ID NO: 283 - protein, 12 aa, Artificial Sequence
  Description of Artificial Sequence Synthetic peptide
ADYDFWSGYFDY

SEQ ID NO: 284 - protein, 15 aa, Artificial Sequence
  Description of Artificial Sequence Synthetic peptide
DGYDSSGYYHGYFDY

SEQ ID NO: 285 - protein, 12 aa, Artificial Sequence
  Description of Artificial Sequence Synthetic peptide
ADYDFWNGYFDY

SEQ ID NO: 286 - protein, 8 aa, Artificial Sequence
  Description of Artificial Sequence Synthetic peptide
GWNYVFDY

SEQ ID NO: 287 - protein, 7 aa, Artificial Sequence
  Description of Artificial Sequence Synthetic peptide
EDDGMDV

SEQ ID NO: 288 - protein, 14 aa, Artificial Sequence
  Description of Artificial Sequence Synthetic peptide
DGDVDTAMVDAFDI

SEQ ID NO: 289 - protein, 12 aa, Artificial Sequence
  Description of Artificial Sequence Synthetic peptide
GDYDFWSGEFDY

SEQ ID NO: 290 - protein, 19 aa, Artificial Sequence
  Description of Artificial Sequence Synthetic peptide
DQGQDGYSYGYGYYYGMDV

SEQ ID NO: 291 - protein, 12 aa, Artificial Sequence
  Description of Artificial Sequence Synthetic peptide
ADYDFWSGYFDF

SEQ ID NO: 292 - protein, 12 aa, Artificial Sequence
  Description of Artificial Sequence Synthetic peptide
ADYDFWSGYFDS

SEQ ID NO: 293 - protein, 17 aa, Artificial Sequence
  Description of Artificial Sequence Synthetic peptide
DRLCTNGVCYEDYGMDV

SEQ ID NO: 294 - protein, 13 aa, Artificial Sequence
  Description of Artificial Sequence Synthetic peptide
DVQDYGDYDYFDY

SEQ ID NO: 295 - protein, 12 aa, Artificial Sequence
  Description of Artificial Sequence Synthetic peptide
ADYDFWSGHFDC

SEQ ID NO: 296 - protein, 14 aa, Artificial Sequence
  Description of Artificial Sequence Synthetic peptide
ERYSGYDDPDGFDI

SEQ ID NO: 297 - protein, 12 aa, Artificial Sequence
  Description of Artificial Sequence Synthetic peptide
ADYDFWSGHFDY

SEQ ID NO: 298 - protein, 13 aa, Artificial Sequence
  Description of Artificial Sequence Synthetic peptide
DPHDYSNYEAFDF

SEQ ID NO: 299 - protein, 9 aa, Artificial Sequence
  Description of Artificial Sequence Synthetic peptide
RDEVRGFDY

SEQ ID NO: 300 - protein, 6 aa, Artificial Sequence
  Description of Artificial Sequence Synthetic peptide
GQWLDV

SEQ ID NO: 301 - protein, 15 aa, Artificial Sequence
  Description of Artificial Sequence Synthetic peptide
DRELELYYYYYGMDV

SEQ ID NO: 302 - protein, 15 aa, Artificial Sequence
  Description of Artificial Sequence Synthetic peptide
DRELEGYSNYYGVDV

SEQ ID NO: 303 - protein, 9 aa, Artificial Sequence
  Description of Artificial Sequence Synthetic peptide
HENYGDYNY

SEQ ID NO: 304 - protein, 17 aa, Artificial Sequence
  Description of Artificial Sequence Synthetic peptide
DREREWDDYGDPQGMDV

SEQ ID NO: 305 - protein, 16 aa, Artificial Sequence
  Description of Artificial Sequence Synthetic peptide
FGDLPYDYSYYEWFDP

SEQ ID NO: 306 - protein, 17 aa, Artificial Sequence
  Description of Artificial Sequence Synthetic peptide
DLYDFWSGYPYYYGMDV

SEQ ID NO: 307 - protein, 16 aa, Artificial Sequence
  Description of Artificial Sequence Synthetic peptide
DYYGSGSFYYYYGMDV

SEQ ID NO: 308 - protein, 16 aa, Artificial Sequence
  Description of Artificial Sequence Synthetic peptide
EAIFGVGPYYYYGMDV

SEQ ID NO: 309 - protein, 17 aa, Artificial Sequence
  Description of Artificial Sequence Synthetic peptide
GGRYSSSWSYYYYGMDV

SEQ ID NO: 310 - protein, 17 aa, Artificial Sequence
  Description of Artificial Sequence Synthetic peptide
GGDSNYEDYYYYYGMDV

SEQ ID NO: 311 - protein, 19 aa, Artificial Sequence
  Description of Artificial Sequence Synthetic peptide
DSSYYDSSGYYLYYYAMDV

SEQ ID NO: 312 - protein, 15 aa, Artificial Sequence
  Description of Artificial Sequence Synthetic peptide
GGTGTNYYYYYGMDV

SEQ ID NO: 313 - protein, 10 aa, Artificial Sequence
  Description of Artificial Sequence Synthetic peptide
DRGDFDAFDI

SEQ ID NO: 314 - protein, 12 aa, Artificial Sequence
  Description of Artificial Sequence Synthetic peptide
AARLDYYYGMDV

SEQ ID NO: 315 - protein, 9 aa, Artificial Sequence
  Description of Artificial Sequence Synthetic peptide
DKWTWYFDL

SEQ ID NO: 316 - protein, 16 aa, Artificial Sequence
  Description of Artificial Sequence Synthetic peptide
DSESEYSSSSNYGMDV

SEQ ID NO: 317 - protein, 12 aa, Artificial Sequence
  Description of Artificial Sequence Synthetic peptide
QMAGNYYYGMDV

SEQ ID NO: 318 - protein, 11 aa, Artificial Sequence
  Description of Artificial Sequence Synthetic peptide
RASQGIRNDLG

SEQ ID NO: 319 - protein, 17 aa, Artificial Sequence
  Description of Artificial Sequence Synthetic peptide
KSSQSVLYSSNNKNYLA

SEQ ID NO: 320 - protein, 11 aa, Artificial Sequence
  Description of Artificial Sequence Synthetic peptide
RASQDIRNDLG

SEQ ID NO: 321 - protein, 16 aa, Artificial Sequence
  Description of Artificial Sequence Synthetic peptide
RSSQSLLHSNGYNYLD

SEQ ID NO: 322 - protein, 16 aa, Artificial Sequence
  Description of Artificial Sequence Synthetic peptide
RSSQSLLHSNGYNYLE

SEQ ID NO: 323 - protein, 12 aa, Artificial Sequence
  Description of Artificial Sequence Synthetic peptide
RASQSLSGNYLA

SEQ ID NO: 324 - protein, 11 aa, Artificial Sequence
  Description of Artificial Sequence Synthetic peptide
QASQDISNYLN

SEQ ID NO: 325 - protein, 11 aa, Artificial Sequence
  Description of Artificial Sequence Synthetic peptide
RAGQGIRNDLG

SEQ ID NO: 326 - protein, 11 aa, Artificial Sequence
  Description of Artificial Sequence Synthetic peptide
RASQSISSYLN

SEQ ID NO: 327 - protein, 11 aa, Artificial Sequence
  Description of Artificial Sequence Synthetic peptide
RASQGIRDDLG

SEQ ID NO: 328 - protein, 11 aa, Artificial Sequence
  Description of Artificial Sequence Synthetic peptide
QASQDISNYLS

SEQ ID NO: 329 - protein, 16 aa, Artificial Sequence
  Description of Artificial Sequence Synthetic peptide
RSSQSLVYSDGYTYLH

SEQ ID NO: 330 - protein, 16 aa, Artificial Sequence
  Description of Artificial Sequence Synthetic peptide
RSSQSLLYSNGYNYLD

SEQ ID NO: 331 - protein, 11 aa, Artificial Sequence
  Description of Artificial Sequence Synthetic peptide
RASQAISNYLN

SEQ ID NO: 332 - protein, 11 aa, Artificial Sequence
  Description of Artificial Sequence Synthetic peptide
RASQSIRSYLN

SEQ ID NO: 333 - protein, 11 aa, Artificial Sequence
  Description of Artificial Sequence Synthetic peptide
RASQSISSYLH

SEQ ID NO: 334 - protein, 16 aa, Artificial Sequence
  Description of Artificial Sequence Synthetic peptide
KSSQSLLLSDGGTYLY

SEQ ID NO: 335 - protein, 11 aa, Artificial Sequence
  Description of Artificial Sequence Synthetic peptide
RASQSISIYLH

SEQ ID NO: 336 - protein, 12 aa, Artificial Sequence
  Description of Artificial Sequence Synthetic peptide
RASQSVSSSYLA

SEQ ID NO: 337 - protein, 11 aa, Artificial Sequence
  Description of Artificial Sequence Synthetic peptide
QASQDITNYLN

SEQ ID NO: 338 - protein, 16 aa, Artificial Sequence
  Description of Artificial Sequence Synthetic peptide
RSSQSLLYSNGYKYLD

SEQ ID NO: 339 - protein, 11 aa, Artificial Sequence
  Description of Artificial Sequence Synthetic peptide
RASQSINSYLN

SEQ ID NO: 340 - protein, 17 aa, Artificial Sequence
  Description of Artificial Sequence Synthetic peptide
RSSQSVLYSSSNRNYLA

SEQ ID NO: 341 - protein, 11 aa, Artificial Sequence
  Description of Artificial Sequence Synthetic peptide
RASQTISSYLN

SEQ ID NO: 342 - protein, 12 aa, Artificial Sequence
  Description of Artificial Sequence Synthetic peptide
RASQSVISIYLA

SEQ ID NO: 343 - protein, 7 aa, Artificial Sequence
  Description of Artificial Sequence Synthetic peptide
AASSLQS

SEQ ID NO: 344 - protein, 7 aa, Artificial Sequence
  Description of Artificial Sequence Synthetic peptide
WASTRES

SEQ ID NO: 345 - protein, 7 aa, Artificial Sequence
  Description of Artificial Sequence Synthetic peptide
LDSHRAS

SEQ ID NO: 346 - protein, 7 aa, Artificial Sequence
  Description of Artificial Sequence Synthetic peptide
LGSNRAS

SEQ ID NO: 347 - protein, 7 aa, Artificial Sequence
  Description of Artificial Sequence Synthetic peptide
GASSRAT

SEQ ID NO: 348 - protein, 7 aa, Artificial Sequence
  Description of Artificial Sequence Synthetic peptide
VASNLET

SEQ ID NO: 349 - protein, 7 aa, Artificial Sequence
  Description of Artificial Sequence Synthetic peptide
AASRLQS

SEQ ID NO: 350 - protein, 7 aa, Artificial Sequence
  Description of Artificial Sequence Synthetic peptide
AASSLQN

SEQ ID NO: 351 - protein, 7 aa, Artificial Sequence
  Description of Artificial Sequence Synthetic peptide
DASNLET

SEQ ID NO: 352 - protein, 7 aa, Artificial Sequence
  Description of Artificial Sequence Synthetic peptide
AESSLQS

SEQ ID NO: 353 - protein, 7 aa, Artificial Sequence
  Description of Artificial Sequence Synthetic peptide
KVSNWDS

SEQ ID NO: 354 - protein, 7 aa, Artificial Sequence
  Description of Artificial Sequence Synthetic peptide
LGFHRAS

SEQ ID NO: 355 - protein, 7 aa, Artificial Sequence
  Description of Artificial Sequence Synthetic peptide
AASNLQS

SEQ ID NO: 356 - protein, 7 aa, Artificial Sequence
  Description of Artificial Sequence Synthetic peptide
EVSNRFS

SEQ ID NO: 357 - protein, 7 aa, Artificial Sequence
  Description of Artificial Sequence Synthetic peptide
GASSWAT

SEQ ID NO: 358 - protein, 7 aa, Artificial Sequence
  Description of Artificial Sequence Synthetic peptide
GASGLQS

SEQ ID NO: 359 - protein, 7 aa, Artificial Sequence
  Description of Artificial Sequence Synthetic peptide
AASSLQG

SEQ ID NO: 360 - protein, 9 aa, Artificial Sequence
  Description of Artificial Sequence Synthetic peptide
LQHNSYPWT

SEQ ID NO: 361 - protein, 9 aa, Artificial Sequence
  Description of Artificial Sequence Synthetic peptide
LQHNGYPWT

SEQ ID NO: 362 - protein, 9 aa, Artificial Sequence
  Description of Artificial Sequence Synthetic peptide
QQYYSTPLT

SEQ ID NO: 363 - protein, 9 aa, Artificial Sequence
  Description of Artificial Sequence Synthetic peptide
LQHNNYPWT

SEQ ID NO: 364 - protein, 9 aa, Artificial Sequence
  Description of Artificial Sequence Synthetic peptide
LQHNTYPWT

SEQ ID NO: 365 - protein, 9 aa, Artificial Sequence
  Description of Artificial Sequence Synthetic peptide
MQALQTPLT

SEQ ID NO: 366 - protein, 9 aa, Artificial Sequence
  Description of Artificial Sequence Synthetic peptide
MQALQTPIT

SEQ ID NO: 367 - protein, 9 aa, Artificial Sequence
  Description of Artificial Sequence Synthetic peptide
QQYDRSPLT

SEQ ID NO: 368 - protein, 9 aa, Artificial Sequence
  Description of Artificial Sequence Synthetic peptide
QQCDNLPLT

SEQ ID NO: 369 - protein, 9 aa, Artificial Sequence
  Description of Artificial Sequence Synthetic peptide
QHYDTLPLT

SEQ ID NO: 370 - protein, 9 aa, Artificial Sequence
  Description of Artificial Sequence Synthetic peptide
QQSYSTPIT

SEQ ID NO: 371 - protein, 9 aa, Artificial Sequence
  Description of Artificial Sequence Synthetic peptide
LQHHSYPWT

SEQ ID NO: 372 - protein, 9 aa, Artificial Sequence
  Description of Artificial Sequence Synthetic peptide
QQYDNPPCS

SEQ ID NO: 373 - protein, 9 aa, Artificial Sequence
  Description of Artificial Sequence Synthetic peptide
MQGAHWPIT

SEQ ID NO: 374 - protein, 9 aa, Artificial Sequence
  Description of Artificial Sequence Synthetic peptide
RQALQTPLT

SEQ ID NO: 375 - protein, 9 aa, Artificial Sequence
  Description of Artificial Sequence Synthetic peptide
QQNNSLPIT

SEQ ID NO: 376 - protein, 9 aa, Artificial Sequence
  Description of Artificial Sequence Synthetic peptide
QQSNGSPLT

SEQ ID NO: 377 - protein, 9 aa, Artificial Sequence
  Description of Artificial Sequence Synthetic peptide
QQSYSNPLT

SEQ ID NO: 378 - protein, 9 aa, Artificial Sequence
  Description of Artificial Sequence Synthetic peptide
QQSISSPLT

SEQ ID NO: 379 - protein, 9 aa, Artificial Sequence
  Description of Artificial Sequence Synthetic peptide
QQSYSTPLT

SEQ ID NO: 380 - protein, 9 aa, Artificial Sequence
  Description of Artificial Sequence Synthetic peptide
QQSYSTPRT

SEQ ID NO: 381 - protein, 9 aa, Artificial Sequence
  Description of Artificial Sequence Synthetic peptide
MQSMQLPIT

SEQ ID NO: 382 - protein, 9 aa, Artificial Sequence
  Description of Artificial Sequence Synthetic peptide
QQSYTSPIT

SEQ ID NO: 383 - protein, 9 aa, Artificial Sequence
  Description of Artificial Sequence Synthetic peptide
QQYYTTPLT

SEQ ID NO: 384 - protein, 9 aa, Artificial Sequence
  Description of Artificial Sequence Synthetic peptide
QQYGSSPLT

SEQ ID NO: 385 - protein, 9 aa, Artificial Sequence
  Description of Artificial Sequence Synthetic peptide
QQCENFPIT

SEQ ID NO: 386 - protein, 9 aa, Artificial Sequence
  Description of Artificial Sequence Synthetic peptide
QQSYSSPLT

SEQ ID NO: 387 - protein, 9 aa, Artificial Sequence
  Description of Artificial Sequence Synthetic peptide
QQYYSTPRT

SEQ ID NO: 388 - protein, 9 aa, Artificial Sequence
  Description of Artificial Sequence Synthetic peptide
QQYGSSPCS

SEQ ID NO: 389 - DNA, 5511 bp, Homo sapiens
  CDS (194)..(4219)
acacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatcca

SEQ ID NO: 390 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT